(19) United States
(12) Patent Application Publication
(10) Pub. No.: US 2024/0000927 A1
(43) Pub. Date: Jan. 4, 2024

(54) IMMUNOMODULATORY AGENT

(71) Applicant: THE QUEEN'S UNIVERSITY OF BELFAST, Belfast (GB)

(72) Inventor: Brian Desmond GREEN, Belfast (GB)

(21) Appl. No.: 18/034,651

(22) PCT Filed: Oct. 29, 2021
(86) PCT No.: PCT/GB2021/052818

§ 371 Date: Apr. 28, 2023

(30) Foreign Application Priority Data
Oct. 30, 2020 (GB) 2017255.7

(51) Int. Cl. A61K 39/39; C08H 8/00; A61P 37/04

(52) U.S. Cl. CPC A61K 39/39; A61K 2039/55583; A61P 37/04; C08H 8/00

(57) ABSTRACT

This invention relates to a polysaccharide that can be used to modulate immune responses.

### FIELD OF THE INVENTION

The invention relates to a polysaccharide, a method of isolating the polysaccharide, a composition comprising the polysaccharide, the polysaccharide for use as a medicament, a method of treatment using the polysaccharide and other specific medical uses of the polysaccharide.

### BACKGROUND

The innate immune system acts as the first line of defence once a microbe has entered into an animal or human body. The adaptive immune system is the second line of defence, and is brought into action when the innate immune system is dealing with a microbe, especially if it is unable to remove the microbe. Unlike the innate immune system, the adaptive immune system shows specificity towards unique antigen(s) present on an invading microbe and it remembers the unique microbial antigen even after it has been cleared from the body. These characteristics of the adaptive immune system enable it to expel unique antigen(s) (and the associated microbe) more rapidly on its second or third time of entry into the body, and also form the basis on which vaccines work.

Vaccines generally, and at the very least, contain an antigen, which primes the adaptive immune system. Therefore, if a live microbe with the same antigen is subsequently encountered, memory B cells and memory T cells of the adaptive immune system (which remember the antigen, and were formed from naïve T cells after the first encounter with the antigen) coordinate a specific and more effective immune response so that the antigen/microbe is cleared quicker.

Mature B cells (B lymphocytes) generally function by producing antibodies against extracellular microbes, whereas mature T lymphocytes generally help kill intracellular microbes. CD8+ T cells, also known as cytotoxic T cells, kill intracellular microbes by binding to infected cells and releasing cytotoxic agents. CD4+ T cells, also known as T helper cells (T_{H}), provide protection against intracellular microbes by producing cytokines that either induce the activation of an inflammatory response (through the activation of macrophages) or induce the activation of a humoral response (by activating B cells). T helper cells that induce the activation of an inflammatory response are referred to as T_{H}1 cells, whereas T helper cells that induce the activation of a humoral response are referred to as T_{H}2 cells.

Unfortunately, some vaccines cannot prime the adaptive immune system enough to enable it to form an adequate response against a microbe. Alternatively or additionally, some vaccines would require large amounts of foreign microbial material in order to induce an immune response. However, if too much microbial material is included in a vaccine, it could be detrimental to the user. As a result of these and other problems, some vaccines are administered with an immunomodulatory agent (e.g. an adjuvant) in order to potentiate the effect of the vaccine. In order for an adjuvant to be effective, it must be able to potentiate the relevant adaptive immune response (e.g. T_{H}1-mediated immune responses or T_{H}2-mediated immune responses).

There is therefore a need for an immunomodulatory agent that can be used to modulate adaptive immune responses.

### STATEMENTS OF THE INVENTION

According to a first aspect of the invention, there is provided a polysaccharide comprising “n” repeating units, wherein each of the “n” repeating units comprises a backbone of alpha-(1-5)-linked arabinofuranose residues, a first side chain of a single alpha-arabinofuranose residue (1-2)-linked to an arabinofuranose residue of the backbone, and a second side chain of a single alpha-arabinofuranose residue (1-3)-linked to the same arabinofuranose residue of the backbone.

The inventors have found that extracts of various plants exhibit immunomodulatory properties (see Example 3). In Example 4, it is confirmed that a novel polysaccharide is responsible for the observed immunomodulatory activity. Advantageously, therefore, the polysaccharide according to the invention may be used to modulate (i.e. stimulate and/or attenuate) an immune response. As shown in the Examples, the polysaccharide is particularly effective at inducing T_{H}1-mediated immune responses. However, given that T_{H}1 responses and T_{H}2 responses are counteractive, it will be appreciated that the polysaccharide of the invention may be used to attenuate or prevent T_{H}2-mediated immune responses. Thus the polysaccharide according to the invention may be used to stimulate and/or enhance immune responses as well as stop and/or attenuate immune responses. The polysaccharide is also advantageous because it is edible, non-toxic and efficacious after oral consumption, and does not induce anaphylaxis. Therefore, it may be administered orally.

The polysaccharide according to the invention may be capable of one or more of:
inducing or enhancing the release of T_{H}1 cytokines (e.g. IFNγ, TNFα and/or IL-2);
inducing or enhancing proliferation of lymphocytes (such as T cells and/or B cells);
inducing or enhancing production of IgM, IgA and/or IgG antibodies;
activating or enhancing activation of macrophages (e.g. activating iNOS/releasing NO and/or releasing O2-);
inducing or enhancing phagocytosis by macrophages;
having a prebiotic effect (stimulating the growth and/or activity of advantageous bacteria or fungi colonising the intestine by acting as a substrate for them);
inducing or enhancing the release of IL-6 and/or TNFα, particularly in macrophages;
inducing or enhancing the release of IL-6 and/or IL-12, particularly in microglia;
inducing or enhancing the release of IL-9, IL-6 and/or IL-71A by immune cells;
inducing or enhancing the production of IL-8 in immune cells; and
inhibiting or suppressing the production of MIG (monokine induced by gamma) in immune cells.

Thus, the polysaccharide according to the invention may be capable of treating, preventing or ameliorating an infection by a foreign body or microorganism, such as a bacterium, a fungi and/or a virus.

The polysaccharide according to the invention or may be capable of one or more of the following:
attenuating or preventing the release of T_{H}2 cytokines (e.g. IL-4, IL-5, IL-9, IL-10, IL-13 and/or IL-25);
preventing or attenuating B cell class switching to IgE;
preventing or attenuating the release of IgE antibodies;
preventing or attenuating mast cell degranulation;
preventing or inhibiting basophil degranulation;
preventing or inhibiting eosinophilia; and
preventing or inhibiting activation of dendritic cells.

The polysaccharide of the invention may be isolated from a Malvales plant. The polysaccharide of the invention may be isolated from a plant of the Malvales order. The Malvales order comprises the Bixaceae family, the Cistaceae family, the Cytinaceae family, the Dipterocarpaceae family, the Muntingiaceae family, the Neuradaceae family, the Sarcloaenaceae family, the Sphaerosepalaceae family, the Thymelaeceae family and the Malvaceae family. Preferably the plant is a member of the Malvaceae family. The Malvaceae family comprises the subfamilies Bombacoideae, Brownlowioideae, Bytnnerioideae, Byttnerioideae, Dombeyoideae, Grewioideae, Helicteroideae, Malvoideae, Sterculioideae and Tiliodeae. Preferably the plant is a Malvoideae. The Malvoideae comprises the tribes Malveae, Gossypieae, Hibisceae, Kydieae. Preferably the plant is from the Malveae tribe. The plant may be from the Sida genus or the Malva genus or the Malvastrum genus or the Sidalcea genus or the Abutilon genus or the Althea genus or the Sphaeralcea genus or the Lavatera genus. Most preferably the plant of the Sida genus is Sida cordifolia (also referred to as ilima, flannel weed, bala, country mallow or heart-leaf sida). Most preferably the plant of the Malva genus is Malva sylvestris. Most preferably the plant of the Malvastrum genus is Malvastrum lateritium. Most preferably the plant of the Sidalcea genus is Sidalcea malviflora. Most preferably plant of the Abutilon genus is Abutilon theophrasti. Most preferably the plant of Althea genus is Althea officinalis. Most preferably the plant of Sphaeralcea genus is Sphaeralcea coccinea. Most preferably the plant of Lavatera genus is Lavatera arborea. Most preferably the plant is Sida cordifolia.

Thus, the polysaccharide may be isolated from a plant of the Sida genus (e.g. Sida cordifolia) or the Malva genus (e.g. Malva sylvestris) or the Malvastrum genus (e.g. Malvastrum lateritium) or the Sidalcea genus (e.g. Sidalcea malviflora) or the Althea genus (e.g. Althea officinalis), or the Sphaeralcea genus (e.g. Sphaeralcea coccinea), or the Lavatera genus (e.g. Lavatera arborea).

Preferably the polysaccharide is a polysaccharide isolated from Sida cordifolia. The polysaccharide may be isolated from part of the plant, such as the leaves, the flower, the stem and/or the roots of the plant. Preferably, the polysaccharide is isolated from the roots of the plant of the Malvales order. Thus, the polysaccharide may be isolated from the roots of a Sida spp. (e.g. Sida cordifolia) or a Malva spp. (e.g. Malva sylvestris) or a Malvastrum spp. (e.g. Malvastrum lateritium) or a Sidalcea spp. (e.g. Sidalcea malviflora). Most preferably the polysaccharide is isolated from the roots of Sida cordifolia.

The polysaccharide according to the invention may be a homopolysaccharide or a heteropolysaccharide. The polysaccharide may be an arabinan polysaccharide. The polysaccharide may be an arabinan homopolysaccharide. The residues of the homopolysaccharide may be L-arabinofuranose residues or be D-arabinofuranose residues. Preferably the residues are L-arabinofuranose residues.

Each repeating unit of the backbone of the polysaccharide according to the first aspect may further comprise 3 or more, 4 or more, 5 or more, 6 or more, 7 or more, 8 or more, 9 or more or 10 additional residues at a terminal residue of the backbone. Preferably the additional residues of the backbone are alpha-(1-5)-linked arabinofuranose residues. More preferably the additional residues of the backbone are alpha-L-(1-5)-linked arabinofuranose residues. One or more, two or more, three or more, four or more, five or more, six or more, seven or more, eight or more or nine or more of the additional alpha-(1-5)-linked arabinofuranose residues may or may not comprise any side chains. One or more of the additional alpha-(1-5)-linked arabinofuranose residues may comprise a side chain of a single alpha-(1-2)-linked arabinofuranose residue. One or more of the additional alpha-(1-5)-linked arabinofuranose residues may comprise a side chain of a single alpha-(1-3)-linked arabinofuranose residue. One, two or three of the additional alpha-(1-5)-linked arabinofuranose residues may comprise a side chain of a single alpha-(1-2)-linked arabinofuranose residue and a side chain of a single alpha-arabinofuranose residue (1-3)-linked to the same additional arabinofuranose residue of the backbone.

Each of the repeating units may independently be branched or unbranched. Thus, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% of the repeating units may be branched. Thus, less than about 100%, about 90%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10% or about 5% of the repeating units may be branched.

Each of the repeating units may be directly or indirectly linked to each other. Preferably each of the repeating units is directly linked to each other via a glycosidic bond, such as an alpha-(1-5) glycosidic bond or an alpha-L-(1-5) glycosidic bond.

The first side chain and the second side chain of the polysaccharide according to the invention may be linked to the same arabinofuranose residue of the backbone.

In one embodiment, the repeating unit comprises or consists of Formula (I) (which may also be referred to herein as block “A”), defined herein as follows:

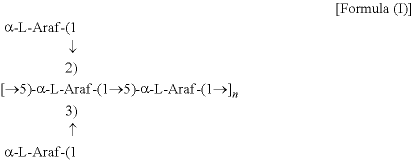

The repeating units of the polysaccharide according to the invention may comprise or consist of blocks referred to herein as blocks “A”, “E” and “F”. Thus, the repeating units of the polysaccharide may further comprise block E and/or block F.

The repeating units may further comprise block E, which may be represented by Formula (II), as follows:

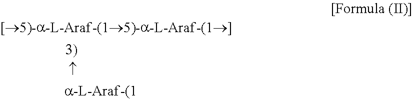

The repeating units may further comprise block F, which may be represented by Formula (III), as follows:

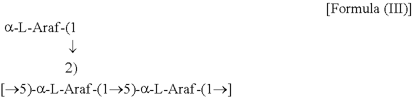

Each block (i.e. block A, block E and block F) comprises a backbone. Each block may be linked by an alpha-(1-5)-glycosidic bond, specifically the backbone of each block. Thus, the repeating units may comprise Formula (I) linked to Formula (II) by an alpha-(1-5)-glycosidic bond. The repeating units may comprise Formula (I) linked to Formula (III) by an alpha-(1-5)-glycosidic bond. The repeating units may comprise Formula (II) linked to Formula (III) by an alpha-(1-5)-glycosidic bond.

Preferably block F (if present in the repeating unit) is the least abundant block of A, E and F. Preferably block A is the most abundant block of A, E and F. Block F (if present in the repeating unit) may be about two, about three, about four, about five, about six, about seven or about less abundant than block A. Block F (if present) may be between about one and about two times less abundant than block A. Preferably for every occurrence of F there is between about 4 and about 6 occurrences of A, and for every occurrence of F there is between about 1 and about 2 occurrences of E. Thus, the ratio of A:E:F in the repeating units may be about 2-8:1-3:1 or about 3-7:1-2:1 or about 4-6:1-2:1. Most preferably the ratio of A:E:F is about 4-6:1-2:1. The ratio of A:F in the repeating units may be about 2-8:1 or about 3-7:1 or about 4-6:1. Most preferably the ratio of A:F is about 4-6:1. The ratio of A:E in the repeating units may be about 2-8:1 or about 2-7:1 or about 2-6:1. Most preferably the ratio of A:F is about 2-6:1.

The repeating unit may comprise Formula (IV), defined herein as follows:

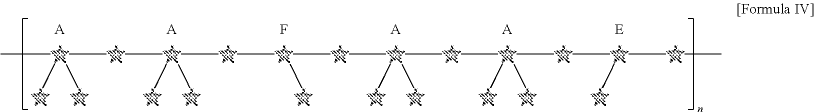

wherein each star of Formula (IV) corresponds to an arabinofuranose, preferably an L-arabinofuranose, more preferably an alpha-arabinofuranose, most preferably an alpha-L-arabinofuranose.

The repeating unit of the polysaccharide according to the invention may be represented by any one of the combinations shown in Table 1, as follows:

| | | | |
|---|---|---|---|
| AAAAE | AAAAF | AAFAAE | AAEAAF |
| AAAEA | AAAFA | AFAAAE | AEAAAF |
| AAEAA | AAFAA | FAAAAE | EAAAAF |
| AEAAA | AFAAA | AAAFEA | AAAEFA |
| EAAAA | FAAAA | AAFAEA | AAEAFA |
| AAAAEF | AAAAFE | AFAAEA | AEAAFA |
| AAAEFA | AAAFEA | FAAAEA | EAAAFA |
| AAAEFA | AAAFEA | AAFEAA | AAEFAA |
| AAEFAA | AAFEAA | AFAEAA | AEAFAA |
| AEFAAA | AFEAAA | FAAEAA | EAAFAA |
| EFAAAA | FEAAAA | AFEAAA | AEFAAA |
| AAAFAE | AAAEAF | FAEAAA | EAFAAA |

Each cell of Table 1 above represents an embodiment of a repeating unit of the polysaccharide according to the invention. Thus, the repeating unit of the polysaccharide may be represented by any one of the 48 examples shown in Table 1.

The polysaccharide of the invention may comprise “n” repeating units, e.g. “n” repeating units of Formula (I) or Formula (IV) or any of the 48 examples shown in Table 1. “n” may be 2 or more, 3 or more, 4 or more, 5 or more 6 or more, 7 or more, 8 or more, 9 or more, 10 or more, 15 or more, 20 or more, 25 or more, 30 or more, 35 or more, or 40 or more, 50 or more, 100 or more, 200 or more, 300 or more, 500 or more, or 1000 or more. “n” may be about 5 to about 1000, about 10 to about 500, or about 15 to about 250, or about 15 to about 230, or about 15 to about 220. Preferably “n” is about 15 to about 220 or about 15 to about 230.

The polysaccharide according to the invention can be used to modulate the immune response of a subject, preferably to stimulate the immune response. The inventors have found that with increasing molecular weight, the modulation of the immune response increases as well per weight amount of polysaccharide. The average molecular weight of the polysaccharide according to the invention or the repeating unit of the polysaccharide according to the invention may be at least about 3 kDa, at least about 5 kDa, at least about 10 kDa, at least about 15 kDa, at least about 20 kDa, at least about kDa, at least about 30 kDa, at least about 35 kDa, at least about 40 kDa, at least about 50 kDa, at least about 60 kDa, at least about 65 kDa, at least about 70 kDa, at least about 75 kDa, at least about 80 kDa, at least about 85 kDa, at least about 90 kDa, at least about 95 kDa, at least about 100 kDa, at least about 105 kDa, at least about 110 kDa, or at least about 115 kDa.

The polysaccharide according to the invention may have an average molecular weight of about 3 kDa to about 200 kDa, 4 kDa to about 180 kDa, 5 kDa to about 160 kDa, 6 kDa to about 140 kDa, about 7 kDa to about 120 kDa, about 8 kDa to about 120 kDa, about 9 kDa to about 120 kDa, about 10 kDa to about 120 kDa, about 20 kDa to about 120 kDa, about 30 kDa to about 120 kDa, about 40 kDa to about 120 kDa, about 50 kDa to about 120 kDa, about 60 kDa to about 120 kDa, about 70 kDa to about 120 kDa, about 80 kDa to about 120 kDa, about 90 kDa to about 120 kDa, about 100 kDa to about 120 kDa or about 110 kDa to about 120 kDa. Preferably the polysaccharide has a molecular weight of 10 kD to 120 kDa.

The polysaccharide of the invention may be administered several different routes, including, for example, oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. Preferably the polysaccharide is administered orally.

The polysaccharide according to the invention is non-toxic, particularly in humans. The polysaccharide according to the invention is non-toxic at doses up to 100 μg/ml. The polysaccharide according to the invention is non-toxic at doses up to 100 μg/ml, particularly in humans.

The polysaccharide according to the invention may or may not be a rhamnogalacturonan, such as rhamnogalacturonan-I of rhamnogalacturonan-II.

According to another aspect, there is provided a method of isolating the polysaccharide according to the invention from a Malvales plant, the method comprising:
i. homogenising and dehydrating the Malvales plant, thereby forming dehydrated Malvales plant particles or powder; and
ii. extracting the polysaccharide from the dehydrated Malvales plant particles and/or powder, thereby isolating the polysaccharide from the Malvales plant.

The method according to the invention may comprise isolating the polysaccharide from a member of the Malvales order or a part of the plant thereof (such as the roots). Thus, the Malvales plant or part thereof may comprise a plant of the Bixaceae family, the Cistaceae family, the Cytinaceae family, the Dipterocarpaceae family, the Muntingiaceae family, the Neuradaceae family, the Sarcloaenaceae family, the Sphaerosepalaceae family, the Thymelaeceae family or the Malvaceae family. Preferably the plant is a member of the Malvaceae family. Thus, the member of the Malvales plant or part thereof may be a subfamily member of the family Malvaceae. The Malvaceae subfamilies comprise Bombacoideae, Brownlowioideae, Bytnnerioideae, Byttnerioideae, Dombeyoideae, Grewioideae, Helicteroideae, Malvoideae, Sterculioideae and Tiliodeae. Preferably the plant is a Malvoideae. The Malvoideae plant may be from the Malveae tribe, the Gossypieae tribe, the Hibisceae tribe or the Kydieae tribe. Preferably the plant is from the Malveae tribe. The plant may be from the Sida genus or the Malva genus or the Malvastrum genus or the Sidalcea genus or the Abutilon genus or the Althea genus or the Sphaeralcea genus or the Lavatera genus. Most preferably the plant of the Sida genus is Sida cordifolia (also referred to as ilima, flannel weed, bala, country mallow or heart-leaf sida). Most preferably the plant of the Malva genus is Malva sylvestris. Most preferably the plant of the Malvastrum genus is Malvastrum lateritium. Most preferably the plant of the Sidalcea genus is Sidalcea malviflora. Most preferably plant of the Abutilon genus is Abutilon theophrasti. Most preferably the plant of Althea genus is Althea officinalis. Most preferably the plant of Sphaeralcea genus is Sphaeralcea coccinea. Most preferably the plant of Lavatera genus is Lavatera arborea. Most preferably the plant is Sida cordifolia.

The polysaccharide may be isolated from a plant of the Sida genus (e.g. Sida cordifolia) or the Malva genus (e.g. Malva sylvestris) or the Malvastrum genus (e.g. Malvastrum lateritium) or the Sidalcea genus (e.g. Sidalcea malviflora) or the Althea genus (e.g. Althea officinalis), or the Sphaeralcea genus (e.g. Sphaeralcea coccinea), or the Lavatera genus (e.g. Lavatera arborea). Preferably the polysaccharide is a Sida cordifolia polysaccharide. The polysaccharide may be isolated from part of Malvales plant, such as the leaves, the flowers, the stem and/or the roots of the plant. Preferably, the polysaccharide is isolated from the roots of the Malvales plant. The polysaccharide may be isolated from the roots of a Sida spp. (e.g. Sida cordifolia) or a Malva spp. (e.g. Malva sylvestris) or a Malvastrum spp. (e.g. Malvastrum lateritium) or a Sidalcea spp. (e.g. Sidalcea malviflora). Most preferably the polysaccharide is isolated from the roots of Sida cordifolia.

The step of homogenising and dehydrating the Malvales plant may comprise dehydrating the plant before homogenising the plant material, or dehydrating the plant after homogenising the plant. Homogenising the Malvales plant increases the surface area and/or reduces the size of the plant, such that the Malvales plant forms particles or a powder. Dehydrating removes moisture from the Malvales plant.

Homogenising the Malvales plant may comprise grinding and/or chopping the plant into particles and/or a powder. Homogenising the plant may comprise using an analytical mill. The particles or powder may be small enough to pass through a sieve size equal to or less than about 0.8 mm.

The step of dehydrating the Malvales plant may comprise lyophilising or heating the plant material to remove moisture. Preferably dehydrating comprises lyophilising.

The step of extracting the polysaccharide from the Malvales plant may comprise one or more from the group consisting of water-alcohol precipitation, dilute alkali leaching, enzyme treatment, microwave extraction, ultrasonic extraction, ultrasonic assisted enzyme extraction, vacuum extraction and pulsed electric field extraction. Preferably the step of extracting the polysaccharide comprises water-alcohol precipitation.

The step of extracting the polysaccharide from the dehydrated Malvales plant particles may comprise one or more alcohol extraction steps (e.g. ethanol extraction steps), which isolate the dehydrated Malvales plant particles or powder into an alcohol phase and a particulate phase, or an aqueous phase into an alcohol phase and a precipitate. The step of extracting the polysaccharide from the dehydrated Malvales plant particles may comprise one or more aqueous extraction steps, which isolate the dehydrated Malvales plant particles into aqueous phase and a precipitate, or which isolate an alcohol phase into an aqueous phase and a precipitate.

The step of extracting the polysaccharide from the dehydrated Malvales plant particles may comprise one or more alcohol extraction steps and/or one or more aqueous extraction steps.

The aqueous extraction step may be performed using an aqueous solution, such as water, saline or other solutions containing a salt (e.g. Phosphate Buffered Saline). The alcohol extraction step(s) may be performed using ethanol. Preferably there are two alcohol extraction steps, most preferably there is a first alcohol extraction step (e.g. an ethanol extraction step), followed by one or more aqueous extraction steps, followed by a second (final) alcohol extraction step (e.g. an ethanol extraction step).

The (first) alcohol extraction step may comprise isolating the dehydrated Malvales plant particles or powder into an alcohol phase and a particulate phase. Preferably the alcohol extraction step is performed for at least about 4, at least about 8 hours or at least about 12 hours, optionally while simultaneously stirring during the entire alcohol extraction step. The alcohol phase and the precipitate may be separated by centrifugation. Centrifugation may be performed at about 6000 rpm to about 7000 rpm, preferably about 6500 rpm. Centrifugation may be performed at under about 0.1 to about 10° C., at about 1° C. to 9° C., at about 2° C. to 8° C., at about 2° C. to 7° C., at about 3° C. to 6° C. or at about 3° C. to 6° C. Preferably centrifugation is performed at about 4° C. Centrifugation may be performed for about 10 minutes or more, about 20 minutes or more, about 30 minutes or more, 1 hour or more. Preferably the centrifugation is performed for about 30 minutes or more. Centrifugation may be performed at about 6000 rpm to about 7000 rpm or at about 6500 rpm, at about 4° C., for about 10 minutes or more, for about 20 minutes or more, for 30 minutes or more. The alcohol phase may be discarded. The particulate phase, which contains the polysaccharide according to the invention, may be kept. The (first) alcohol extraction step may be performed using 95% alcohol (e.g. ethanol) at room temperature.

The one or more aqueous extraction steps may be one or more, two or more, three or more, four or more, five or more, six or more aqueous extraction steps. The one or more aqueous extraction steps may comprise isolating the precipitate of a first alcohol extraction step or an aqueous extraction step into an aqueous phase and a precipitate. The one or more aqueous extraction steps may be performed using a boiling aqueous solution, preferably an aqueous solution that has been maintained at a temperature of about 100° C. (during the entire extraction step). The aqueous extraction step may be performed for about 30 minutes or more, about 1 hour or more, or about 2 hours or more using a boiling aqueous solution, such as water. The aqueous extraction step may comprise stirring during the entire aqueous extraction step. The aqueous phase and the precipitate may be separated by centrifugation. Centrifugation may be performed at about 6000 rpm to about 7000 rpm, preferably about 6500 rpm. Centrifugation may be performed at under about 0.1 to about 10° C., at about 1° C. to 9° C., at about 2° C. to 8° C., at about 2° C. to 7° C., at about 3° C. to 6° C. or at about 3° C. to 6° C. Preferably centrifugation is performed at about 4° C. Centrifugation may be performed for about 10 minutes or more, about 20 minutes or more, about 30 minutes or more, 1 hour or more. Preferably the centrifugation is performed for about 30 minutes or more. Centrifugation may be performed at about 6000 rpm to about 7000 rpm or at about 6500 rpm, at about 4° C., for about 10 minutes or more, for about 20 minutes or more, for 30 minutes or more. The separated aqueous phase of the one or more aqueous extraction steps, which contains the polysaccharide according to the invention, may be kept and pooled together. The precipitate of the aqueous extraction step may be used in a further aqueous extraction step to isolate further polysaccharide according to the invention.

The aqueous phase of the one or more aqueous extraction steps that have been pooled together may be treated with one or more enzymes at room temperature (or at about 37° C.) to digest unwanted carbohydrates and proteins, optionally followed by dialysis. The enzymes may be selected from the group consisting of alpha-amylase, pullulanase and protease K. Dialysis may be performed using water. The dialysis may have a pocket cut-off of about 12-14 kDa.

The second/final alcohol extraction step may be performed on the aqueous phase of the one or more aqueous extraction steps that have been pooled together, such that an alcohol phase and a precipitate are formed. Preferably the second/final alcohol extraction step is performed on the aqueous phase of the one or more aqueous extraction steps that have been pooled together, enzymatically treated and dialysed The (second/final) alcohol extraction step may be performed using 80% alcohol (e.g. 80% ethanol) at room temperature (e.g. about 18-22° C.). Preferably the alcohol extraction step is performed for at least about 4 hours, at least about 8 hours or at least about 12 hours, optionally while stirring during the entire alcohol extraction step. The alcohol phase and the precipitate may be separated by centrifugation. Centrifugation may be performed at about 6000 rpm to about 7000 rpm, preferably about 6500 rpm. Centrifugation may be performed at under about 0.1 to about 10° C., at about 1° C. to 9° C., at about 2° C. to 8° C., at about 2° C. to 7° C., at about 3° C. to 6° C. or at about 3° C. to 6° C. Preferably centrifugation is performed at about 4° C. Centrifugation may be performed for about 10 minutes or more, about 20 minutes or more, about 30 minutes or more, 1 hour or more. Preferably the centrifugation is performed for about 30 minutes or more. Centrifugation may be performed at about 6000 rpm to about 7000 rpm or at about 6500 rpm, at about 4° C., for about 10 minutes or more, for about 20 minutes or more, for 30 minutes or more. The alcohol phase may be discarded. The particulate phase, which contains the polysaccharide according to the invention, may be used or further enriched using chromatographic techniques known in the art. Chromatographic techniques known in the art include ion-exchange chromatography, size-exclusion chromatography, reversed phase chromatography, high performance liquid chromatography and flash chromatography.

The method according to the invention may comprise a final (iii) purification step. The purification step may comprise purifying the polysaccharide isolated from the Malvales plant using a chromatographic technique, a crystallisation technique or a distillation technique.

According to another aspect, there is provided an isolated polysaccharide produced by the method according to the invention.

The polysaccharide according to the invention may be used as a cell culture additive. Therefore, according to another aspect, there is provided a culture media comprising an isolated polysaccharide according to the invention or an isolated polysaccharide produced by the method according to the invention.

The culture media may further comprise a cell, biological tissue, a biological organ, a biological system or an organism.

The culture media may be a cloning medium or a hybridoma feeder supplement (for the purpose of enhancing cell cloning efficiency or hybridoma growth and survival rates).

The polysaccharide according to the invention may be used as an adjunct to, or in combination with, known therapies for treating, ameliorating, or preventing T helper cell-mediated diseases, such as a vaccine, a pharmaceutical composition or an edible composition (e.g. a prebiotic). For example, the polysaccharide of the invention may be used in combination with known agents for treating various allergic (e.g. hay fever), inflammatory (e.g. asthma), and/or autoimmune disorders (e.g. rheumatoid arthritis), such as glucocorticoids, corticosteroids, methotrexate, disease-modifying antirheumatic drugs (DMARDs), Janus kinase (JAK) inhibitors or biopharmaceuticals that interact with cytokines (e.g. Etanercept).

Thus, according to another aspect of the invention, there is provided a composition comprising a polysaccharide according to the invention or an isolated polysaccharide produced by the method according to the invention, optionally the composition is a pharmaceutical composition and a pharmaceutically acceptable carrier or such like, or an edible composition (e.g. a prebiotic).

According to another aspect of the invention, there is provided an immunological adjuvant comprising a polysaccharide according to the invention.

According to another aspect of the invention, there is provided a vaccine comprising an immunological adjuvant according to the invention.

According to another aspect, there is provided a polysaccharide according to the invention, a composition according to the invention, an isolated polysaccharide produced by the method according to the invention, an immunological adjuvant according to the invention or a vaccine according to the invention, for use in a medicament.

According to another aspect there is provided an immunological adjuvant or a vaccine according to the invention, for use in vaccination of a subject.

The immunological adjuvant or the vaccine may be for use in vaccinating a subject with a T helper cell (T_{H})-mediated disease or medical condition. The T helper cell (T_{H})-mediated disease or medical condition may be a T_{H}1-mediated disease or medical condition, or a T_{H}2-mediated disease or medical condition.

According to another aspect, there is provided a polysaccharide according to the invention, an isolated polysaccharide produced by the method according to the invention, a composition according to the invention, an immunological adjuvant according to the invention, or a vaccine according to the invention, for use in treating, ameliorating or preventing a disease or a condition of a subject.

According to another aspect, there is provided a polysaccharide, an isolated polysaccharide produced by the method according to the invention, an immunological adjuvant according to the invention or a vaccine according to the invention, a composition according to the invention, or a plant of the Malvales order or part thereof, for use in treating, ameliorating or preventing a T helper cell (T_{H})-mediated disease or medical condition of a subject.

According to another aspect, there is provided a method of vaccinating a subject, the method comprising administering to the subject, an adjuvant according to the invention or a vaccine according to the invention.

According to another aspect, there is provided a method of treating, ameliorating or preventing a T helper cell (T_{H})-mediated disease or medical condition in a subject, the method comprising administering to the subject (i) a polysaccharide according to the invention, (ii) an isolated polysaccharide produced by the method according to the invention, (iii) a composition according to the invention, (iv) an adjuvant according to the invention, (v) a vaccine according to the invention, or (vi) a plant of the Malvales order or part thereof.

Advantageously, the adjuvant or vaccine according to the invention maybe capable of enhancing the immunomodulatory activity of a subject that received the adjuvant, thus resulting in the stimulation of the immune system for treating conditions in which the T_{H}1-mediated immune response is underactive or the T_{H}2-mediated response is overactive.

The plant may be a plant of the Malvales order or a part of the plant thereof as referred to herein

Thus, the T helper cell (T_{H})-mediated disease or medical condition may be a T_{H}1-mediated disease or medical condition, or a T_{H}2-mediated disease or medical condition.

A T_{H}1-mediated disease or medical condition in which the T_{H}1-mediated response is ineffective or underactive may be one or more selected from the group comprising/consisting of rheumatoid arthritis (RA); psoriatic arthritis; psoriasis; inflammatory bowel syndrome (IBD); Crohn's disease; ulcerative colitis; multiple sclerosis (MS); flu, including pandemic flu; respiratory disorders, for example those caused by viruses, such as respiratory syncytial virus (RSV); cystic fibrosis (CF); herpes, including genital herpes; sepsis and septic shock; bacterial pneumonia; bacterial meningitis; dengue hemorrhagic fever; endometriosis; prostatitis; uveitis; uterine ripening; alopecia areata; ankylosing spondylitis; coeliac disease; dermatomyositis; diabetes mellitus Type 1; Goodpasture's syndrome; Graves' disease; Guillain-Barre syndrome; juvenile idiopathic arthritis; Hashimoto's thyroiditis; idiopathic thrombocytopenic purpura; Lupus erythematosus; mixed connective tissue disease; myasthenia gravis; narcolepsy; osteoarthritis; pemphigus vulgaris; pernicious anaemia; polymyositis; primary biliary cirrhosis; relapsing polychondritis; Sjogren's syndrome; temporal arteritis; vasculitis; Wegener's granulumatosis; age-related macular degeneration, an infectious disease (e.g. infection with Mycobacterium tuberculosis (Mt), human immunodeficiency virus (HIV) or a coronavirus)); an autoimmune disorder (e.g. arthritis, multiple sclerosis or type 1 diabetes); a cancer; post-cancer surgery or cancer treatment; and post-immunisation. Preferably the T_{H}1-mediated disease or medical conditions are one or more of cystic fibrosis (CF), diabetes mellitus Type I, age-related macular degeneration, an infectious disease (e.g. infection with Mycobacterium tuberculosis (Mt), human immunodeficiency virus (HIV) or a coronavirus). The T_{H}1-mediated disease or medical condition may be cystic fibrosis (CF). The T_{H}1-mediated disease or medical condition may be diabetes mellitus Type I. The T_{H}1-mediated disease or medical condition may be age-related macular degeneration. The T_{H}1-mediated disease or medical condition may be an infectious disease. The T_{H}1-mediated disease or medical condition may be a Mycobacterium tuberculosis (Mt) infection. The T_{H}1-mediated disease or medical condition may be infection with human immunodeficiency virus (HIV). The T_{H}1-mediated disease or medical condition may be infection with a coronavirus.

A T_{H}2-mediated disease or medical condition in which the T_{H}2-mediated response is overactive may be one or more selected from the group comprising/consisting of type 1 hypersensitivity disorders, including an allergy (e.g. rhinitis, allergic dermatitis, uticaria), asthma, eczema, hay fever, urticarial, chronic graft-versus-host disease, progressive systemic sclerosis, systemic lupus erythematosus; a chronic lung disease; scleroderma; anaphylaxis; atrophy (e.g. muscle); and transplant rejection. Preferably the T_{H}2-mediated disease or medical conditions are one or more of allergy (e.g. rhinitis, allergic dermatitis, uticaria), asthma, eczema and hay fever. The T_{H}2-mediated disease or medical condition may be allergy. The T_{H}2-mediated disease or medical condition may be rhinitis. The T_{H}2-mediated disease or medical condition may be allergic dermatitis. The T_{H}2-mediated disease or medical condition may be uticaria. The T_{H}2-mediated disease or medical condition may be asthma. The T_{H}2-mediated disease or medical condition may be eczema. The T_{H}2-mediated disease or medical condition may be (seasonal and/or perennial allergic rhinitis) hay fever.

Thus, in one embodiment, there is a polysaccharide according to the invention or an isolated polysaccharide produced by the method according to the invention,
for use in treating, preventing or ameliorating in a subject a selection of T_{H}1-mediated diseases/conditions from the group comprising/consisting of cystic fibrosis (CF), diabetes mellitus Type I, age-related macular degeneration and an infectious disease (e.g. infection with Mycobacterium tuberculosis (Mt), human immunodeficiency virus (HIV) or a coronavirus).

In another one embodiment, there is a polysaccharide according to the invention or an isolated polysaccharide produced by the method according to the invention,
for use in treating, preventing or ameliorating cystic fibrosis (CF).

In another one embodiment, there is a polysaccharide according to the invention or an isolated polysaccharide produced by the method according to the invention,
for use in treating, preventing or ameliorating diabetes mellitus Type I.

In another one embodiment, there is a polysaccharide according to the invention or an isolated polysaccharide produced by the method according to the invention,
for use in treating, preventing or ameliorating age-related macular degeneration.

In another one embodiment, there is a polysaccharide according to the invention or an isolated polysaccharide produced by the method according to the invention,
for use in treating, preventing or ameliorating an infectious disease (e.g. infection with Mycobacterium tuberculosis (Mt), human immunodeficiency virus (HIV) or a coronavirus).

In another one embodiment, there is a polysaccharide according to the invention or an isolated polysaccharide produced by the method according to the invention,
for use in treating, preventing or ameliorating an infection with Mycobacterium tuberculosis (Mt).

In another one embodiment, there is a polysaccharide according to the invention or an isolated polysaccharide produced by the method according to the invention,
for use in treating, preventing or ameliorating an infection with human immunodeficiency virus (HIV).

In another one embodiment, there is a polysaccharide according to the invention or an isolated polysaccharide produced by the method according to the invention,
for use in treating, preventing or ameliorating an infection with a coronavirus.

In another embodiment, there is a polysaccharide according to the invention or an isolated polysaccharide produced by the method according to the invention,
for use in treating, preventing or ameliorating in a subject a selection of T_{H}2-mediated diseases/conditions from the group comprising/consisting of allergy (e.g. rhinitis, allergic dermatitis, uticaria), asthma, eczema and hay fever.

In another one embodiment, there is a polysaccharide according to the invention or an isolated polysaccharide produced by the method according to the invention,
for use in treating, preventing or ameliorating allergy.

In another one embodiment, there is a polysaccharide according to the invention or an isolated polysaccharide produced by the method according to the invention,
for use in treating, preventing or ameliorating rhinitis.

In another one embodiment, there is a polysaccharide according to the invention or an isolated polysaccharide produced by the method according to the invention,
for use in treating, preventing or ameliorating allergic dermatitis.

In another one embodiment, there is a polysaccharide according to the invention or an isolated polysaccharide produced by the method according to the invention,
for use in treating, preventing or ameliorating uticaria.

In another one embodiment, there is a polysaccharide according to the invention or an isolated polysaccharide produced by the method according to the invention,
for use in treating, preventing or ameliorating asthma.

In another one embodiment, there is a polysaccharide according to the invention or an isolated polysaccharide produced by the method according to the invention,
for use in treating, preventing or ameliorating eczema.

In another one embodiment, there is a polysaccharide according to the invention or an isolated polysaccharide produced by the method according to the invention,
for use in treating, preventing or ameliorating hay fever.

A polysaccharide according to the invention, a composition according to the invention, an adjuvant according to the invention, a vaccine according to the invention, a plant according to the invention may be administered orally, by aerosol inhalation (e.g. nasal inhalation or oral inhalation), sublingually, topically, transdermally, parenterally (subcutaneously, intramuscularly or intravenously).

The polysaccharide or composition according to the invention may be administered orally. The polysaccharide or composition according to the invention may be administered by aerosol inhalation (e.g. nasal inhalation or oral inhalation). The polysaccharide or composition according to the invention may be administered sublingually. The polysaccharide or composition according to the invention may be administered topically. The polysaccharide or composition according to the invention may be administered transdermally. The polysaccharide or composition according to the invention may be administered parenterally (subcutaneously, intramuscularly or intravenously). Preferably the polysaccharide or composition according to the invention is administered orally.

The method of treatment according to the invention may comprise administering a therapeutically effective amount of a polysaccharide according to the invention, a composition according to the invention, an adjuvant according to the invention or a vaccine according to the invention.

According to another aspect, there is provided a method of culturing a cell, a biological tissue, a biological organ, a biological system or an organism, the method comprising:
placing a cell, a biological tissue, a biological organ, a biological system or an organism in a culture media, and
placing into the culture media (i) a polysaccharide according to the invention, (ii) an isolated polysaccharide produced by the method according to the invention, (iii) a composition according to the invention, (iv) an adjuvant according to the invention, (v) a vaccine according to the invention, or (vi) a plant of the Malvales order or part thereof.

According to another aspect, there is provided a method of inducing or enhancing a T_{H}1-mediated immune response, the method comprising administering or contacting a polysaccharide according to the invention to/with an organism, a biological system, a biological tissue, a biological organ or a cell in order to induce or enhance a T_{H}1-mediated immune response.

A T_{H}1-mediated immune response may comprise one or more from the group comprising/consisting of: production of T_{H}1 cytokines (e.g. IFNγ, TNFα and/or IL-2); induction of lymphocyte proliferation (such as T cell and/or B cell proliferation); production of IgM, IgA and/or IgG antibodies; activation of macrophages (e.g. activation of iNOS/release of NO, release of O₂—); and induction of phagocytosis by macrophages.

According to another aspect, there is provided a method of preventing or attenuating a T_{H}2-mediated immune response, the method comprising administering or contacting a polysaccharide according to the invention to/with an organism, a biological system, a biological tissue, a biological organ or a cell in order to prevent or attenuate a T_{H}2-mediated immune response.

A T_{H}2-mediated immune response may comprise one or more from the group comprising/consisting of: production of T_{H}2 cytokines (e.g. IL-4, IL-5, IL-9, IL-10, IL-13 and IL-25); induction of B cell class switching to IgE, production of IgE antibodies; mast cell degranulation; basophil degranulation; recruitment of eosinophils; and activation of dendritic cells.

The method according to the invention may be an in vivo method, an ex vivo method or an in vitro method.

Abbreviations: kDa—kilo Dalton; Gal—D-Galactose; GalA—D-Galacturonic acid; Rha—L-Rhamnose; Ara—L-Arabinose; Fuc—L-Fucose; Glc—D-Glucose; GlcA—D-Glucuronic acid. The L- and D-forms of these monomers and the corresponding polymers (e.g. polysaccharides) as indicated here also apply to the monomers and polymers (e.g. polysaccharides) as indicated in the rest of this specification (which may not be abbreviated but written in full).

The term “average” may be the arithmetic mean, the mode or the median. Preferably the average refers to the arithmetic mean.

The polysaccharide according to the invention may be an isolated polysaccharide.

The term “isolated” can refer to a polysaccharide that is no longer in its natural environment. Thus, the term “isolated” can refer to a polysaccharide that has been separated from Malvaceae/Malvoideae/Malveae plant tissue and cells (such as Sida Cordifolia tissue and Sida Cordifolia cells).

A “subject” may be a vertebrate, a mammal, a non-human animal or a domestic animal. Hence, polysaccharide, the adjuvant, the vaccine, the composition and the medicament according to the invention may be used to treat any mammal, for example livestock (e.g. a horse), pets, or maybe used in other veterinary applications. Livestock may be bovine, cow, cattle, sheep, horse, chicken, goat, pig, calf, deer, goose, turkey or rabbit. A domestic animal may be a dog or a cat. Preferably the subject is a mammal. Most preferably the mammal is a human being. An organism can refer to a subject.

Modulating the immune response refers to the activity or ability of the immune system to defend the body is modulated. This may relate to immuno-stimulation or immuno-suppression. The primary task of the immune system is to protect against pathogens such as fungi, bacteria, viruses, protozoa and parasites. In this context, modulating immune response preferably means stimulating the immune response so that it can achieve this function. This may be achieved by activating or enhancing the T_{H}1-mediated response. Suitably, stimulation of the immune response contributes to an enhanced natural defence of the human body. On the other hand, the immune system sometimes mounts an immune response against innocuous substances, like house mite, dust or pollen, resulting in allergy (an overactive T_{H}2-mediated response). In addition, many physiological disorders, like hypercholesterolemia and obesity, result in a low-grade inflammatory status. Immune modulation in the context of abnormal immune responses, like allergy or inflammation, means dampening or counteracting the hypersensitivity immune response. The present invention may relate to the primary task of the stimulating/enhancing immune responses, and/or the task of inhibiting the ‘abnormal’ immune response.

Pharmaceutical compositions according to the invention may further comprise a pharmaceutically acceptable salt or other form thereof. Pharmaceutical compositions according to the invention may comprise one or more pharmaceutically acceptable excipients, such as carriers, diluents, fillers, disintegrants, lubricating agents, binders, colorants, pigments, stabilizers, preservatives, antioxidants, and/or solubility enhancers. Pharmaceutical compositions according to the invention may comprise a pharmaceutically acceptable salt and optionally one or more pharmaceutically acceptable excipients.

The pharmaceutical compositions can be formulated by techniques known in the art. The pharmaceutical compositions can be formulated as dosage forms for oral, parenteral, such as topical, transdermal, intramuscular, intravenous, subcutaneous, intradermal, intraarterial, intracardial, nasal, inhalation or aerosol administration (e.g. nasal or oral inhalation). The pharmaceutical composition may be formulated as a dosage form for oral administration.

In the present context, the term “pharmaceutically acceptable salt” is intended to indicate salts which are not harmful to a patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfgionic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, volume 66, issue 2. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, PA, 1995.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. In addition, the compounds of the invention may form solvates with water or common organic solvents. Such solvates are also encompassed within the scope of the present invention.

The composition may further comprise a buffer system, preservative(s), tonicity agent(s), chelating agent(s), stabilizers and surfactants, which is well known to the skilled person. For convenience reference is made to Remington: The Science and Practice of Pharmacy, 20th edition, 2000. The composition may also further comprise one or more therapeutic agents active against the same disease state.

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Administration of pharmaceutical compositions according to the invention may be through several routes of administration, for example, oral, nasal, rectal, inhalation (e.g. nasal inhalation or oral inhalation), pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

For topical use, sprays, creams, ointments, jellies, gels, inhalants, dermal patches, implants, solutions of suspensions, etc., containing the compounds of the present invention are contemplated. For the purpose of this application, topical applications shall include mouth washes and gargles. Compounds of the invention may be used in wafer technology, wafer technology, such as the biodegradable Gliadel polymer wafer, is useful for brain cancer chemotherapy.

Pharmaceutical compositions for oral administration include solid dosage forms such as hard or soft capsules, tablets, troches, dragees, pills, lozenges, powders and granules and liquid dosage forms for oral administration include solutions, emulsions, aqueous or oily suspensions, syrups and elixirs, each containing a predetermined amount of the active ingredient, and which may include a suitable excipient.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations.

Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc.

The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874 to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or as soft gelatine capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions of the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative, a flavouring agent and a colouring agent. The pharmaceutical composition may be in the form of a sterile injectable aqueous or oleaginous suspension. This suspension may be formulated according to known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as a solvent or suspending medium. For this purpose, any bland fixed oil may be employed using synthetic mono- or diglycerides. In addition, fatty acids such as oleic acid find use in the preparation of injectables.

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the prolactin receptor antagonist in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use.

A medicament includes but is not limited to a composition, such as a composition (e.g. a pharmaceutical composition or an edible composition), a prescription drug, a non-prescription drug, an over the counter medicine, a dietary supplement, a dietary food, a clinical food, an edible product, a tablet, a capsule, a pill, and food products such as beverages or any other suitable food product, and any other composition which is commonly known to the skilled person. Alternatively, the medicament may be an injectable substance or an inhalable substance, such as a nasal spray.

The polysaccharide according to the invention may be added to an edible composition or a pharmaceutical composition in a specific salt form. The edible composition according to the present invention may take any physical form. In particular, it may be a food product, a beverage, a dietary food product, or a clinical food product. It may also be a dietary supplement, in the form of a beverage, a tablet, a capsule, a liquid (e.g. a soup or a beverage, a spread, a dressing or a dessert) or any other suitable form for a dietary supplement. The edible composition may be in a liquid or a spreadable form, it may be a spoonable solid or soft-solid product, or it may be a food supplement. Preferably the edible composition is a liquid product. The edible product may suitably take the form of e.g. a soup, a beverage, a spread, a dressing, a dessert, a bread. The term “spread” as used herein encompasses spreadable products such as margarine, light margarine, spreadable cheese-based products, processed cheese, dairy spreads, and dairy-alternative spreads. Spreads as used herein (oil-in-water or water-in-oil emulsions) may have a concentration of oil and/or fat of between about 5% and 85% by weight, preferably between 10% and 80% by weight, more preferred between 20% and 70% by weight. Preferably the oil and/or fat are from vegetable origin (such as but not limited to sunflower oil, palm oil, rapeseed oil); oils and/or fats of non-vegetable origin may be included in the composition as well (such as but not limited to dairy fats, fish oil). The polysaccharide and thus the edible composition according to the invention may be a prebioitic. A prebioitic may be defined as a composition that induces growth and/or activity of beneficial microorganisms, such as fungi and/or bacteria, colonising the intestine. Growth and/or activity of beneficial microorganism may be increased by the polysaccharide acting as a substrate for the beneficial microorganisms.

The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art. Depot injectable formulations are also contemplated as being within the scope of the present invention.

An “immunological adjuvant” may refer to an agent that potentiates an immune response to antigen or a vaccine.

It will be appreciated that the term “treatment” and “treating” as used herein means the management and care of a subject for the purpose of combating a condition, such as a disease or a disorder. The term is intended to include the full spectrum of treatments for a given condition from which the subject is suffering, including alleviating symptoms or complications, delaying the progression of the disease, disorder or condition, alleviating or relieving the symptoms and complications, and/or to cure or eliminating the disease, disorder or condition as well as to prevent the condition, wherein prevention is to be understood as the management and care of a subject for the purpose of combating the disease, condition, or disorder and includes the administration of the ligand to prevent the onset of the symptoms or complications.

The term “comprising” may refer to “consisting of” or “consisting essentially of”.

All of the embodiments and features described herein (including any accompanying claims, abstract and drawings), and/or all of the steps of any method or process so disclosed, may be combined with any of the above aspects or embodiments in any combination, unless stated otherwise with reference to a specific combinations, for example, combinations where at least some of such features and/or steps are mutually exclusive.

For a better understanding of the invention, and to show embodiments of the invention may be put into effect, reference will now be made, by way of example, to the accompanying drawings, in which:—

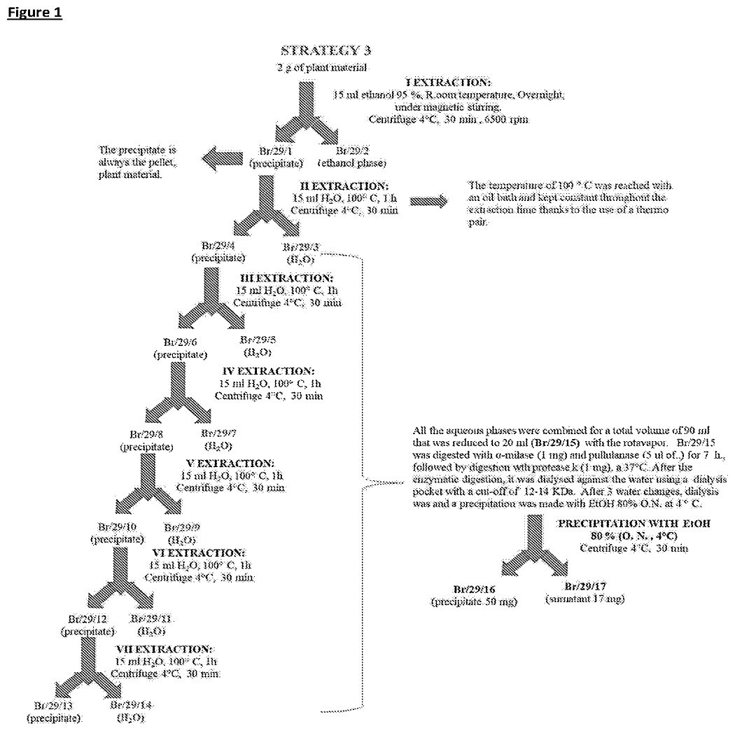

FIG. 1 is a schematic diagram of an optimised extraction protocol for obtaining the active arabinan polysaccharide from plant materials. The flow chart illustrates the 3 main steps required for isolating the polysaccharide including the methodological details. Briefly, the method comprises of a first step involving the use of ethanol to initially extract the polysaccharide followed by a second step involving up to 6 cycles of water (aqueous) extraction, followed by a final enzymatic digestion, dialysis and precipitation step.

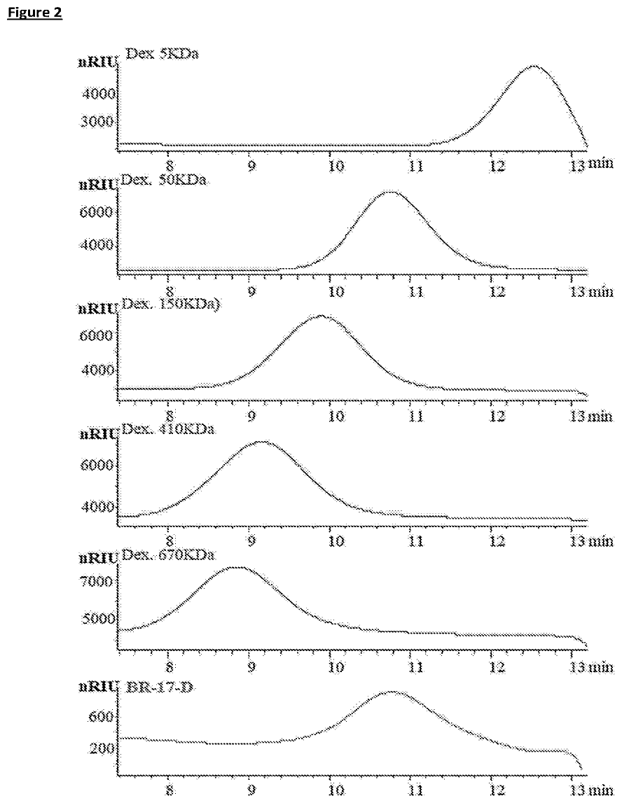

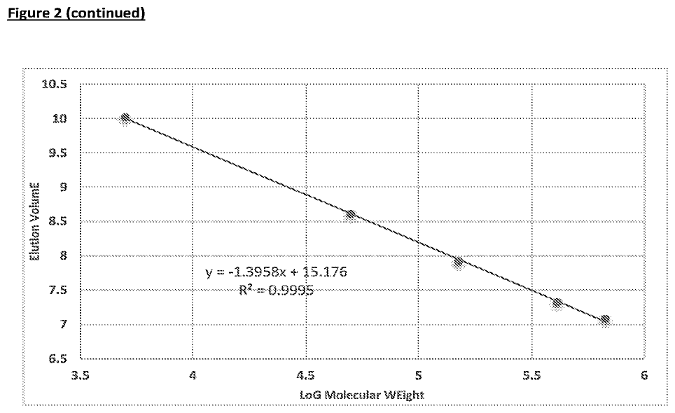

FIG. 2 is an elution profile on Size-Exclusion Chromatography (SEC)-HPLC of dextran at known M.W (in blue) and the arbinan Br/17/D (in red). The comparison with known molecular weight dextrans revealed that the molecular weight of Br/17/D is 49 KDa. Below is an example of a calibration curve based on dextran at known M.W and a typical example of a straight line equation.

FIG. 3 is a linkage analysis of Br/14/E (less pure fraction). GC-MS analysis of partially methylated alditol acetates (PMAA) of the fraction Br/14/E. The top trace shows the chromatogram referred to the abundance of the total ions. The bottom trace (in red): is shown the chromatogram referred to the abundance of the 118 ion (extract chromatogram ion 118), consequently only the peaks with this ion are showed. I=impurity.

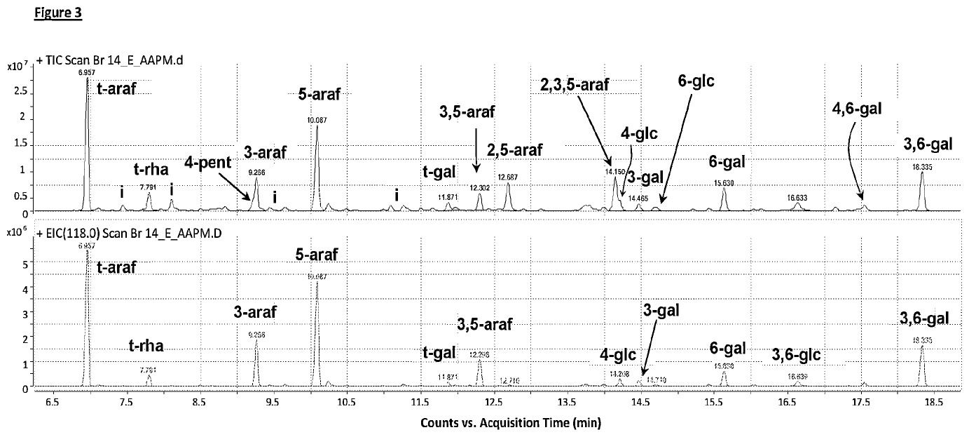

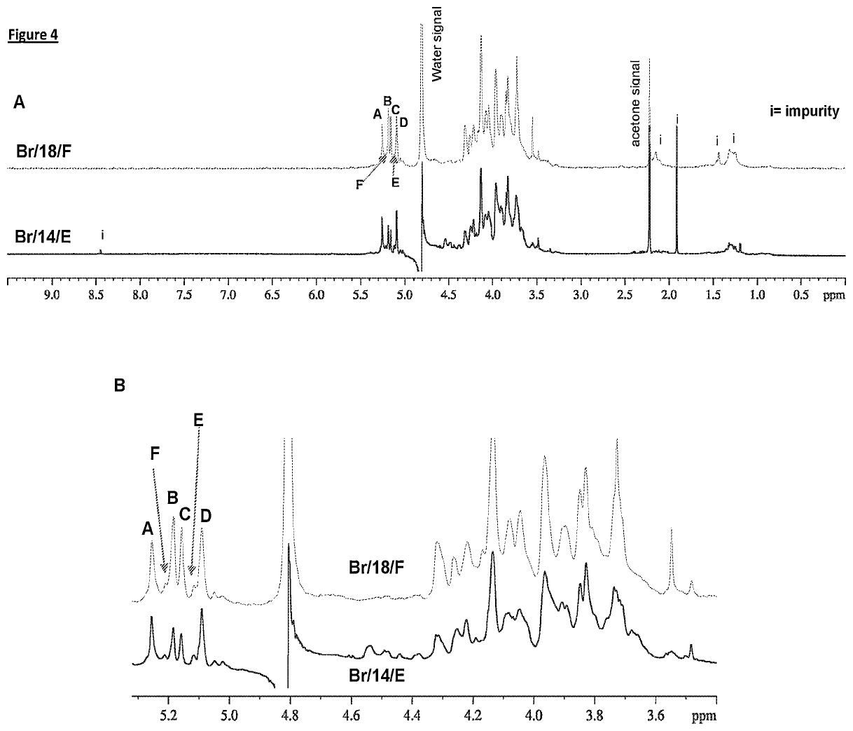

FIG. 4 shows (A) ¹H NMR spectra (600 MHz, 298 K, D₂O) of Br/14/E (less pure fraction) and Br/18/F (pure arabinan); and (B) Expansion of the ¹H NMR spectra of Br/14/E and Br/18/F. The structure of the arabinan is shown in FIG. 57.

FIG. 5 (600 MHz, 298 K, D₂O) A) expansion of the ¹H NMR of the arabinan (Br/18/F) detailing the anomeric region along with the integration values used to evaluate the ratio between the different signals. Each signal is labelled with a capital letter and it is diagnostic of an arabinofuranose unit. B) structure of the arabinan polysaccharide drawn according to the SNFC rules, this structure includes the arabinofuranose units found by NMR analysis and maintains in good approximation the ratios detected by integration.

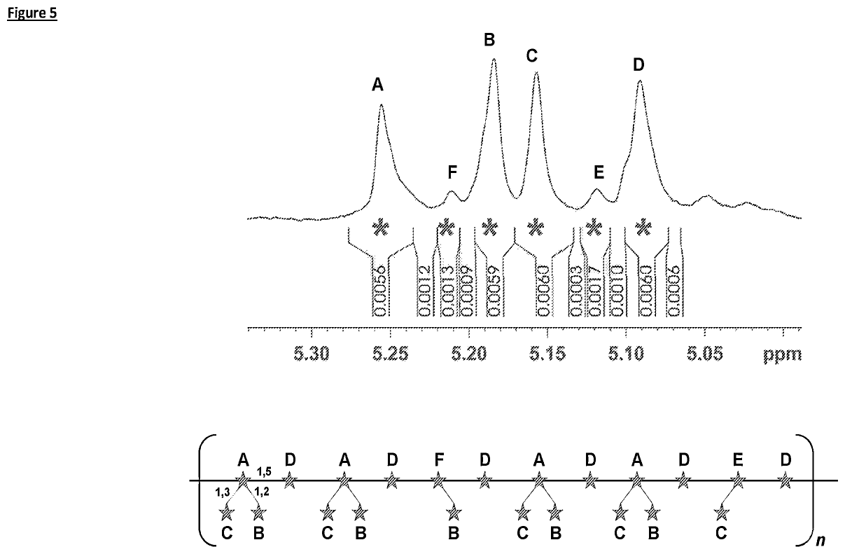

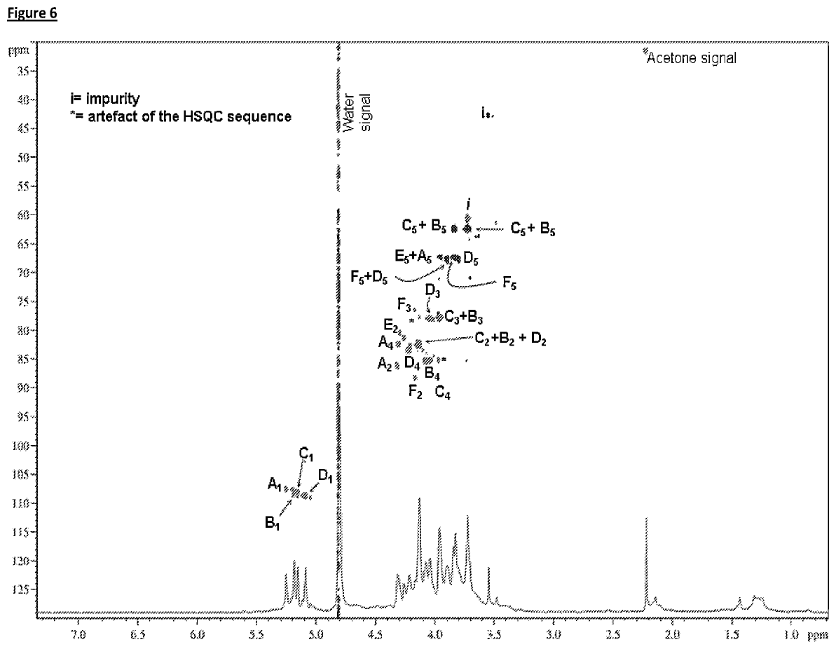

FIG. 6 (600 MHz, 298 K, D₂O) ¹H-¹³C HSQC spectrum drawn at full scale of Br/18/F (pure arabinan with strong activity), densities are labelled for a quick identification, the proton spectrum is reported in red; for the structure of the arabinan and for the labels used refer to FIG. 57B. “i” impurity, * artifact of the HSQC sequence.

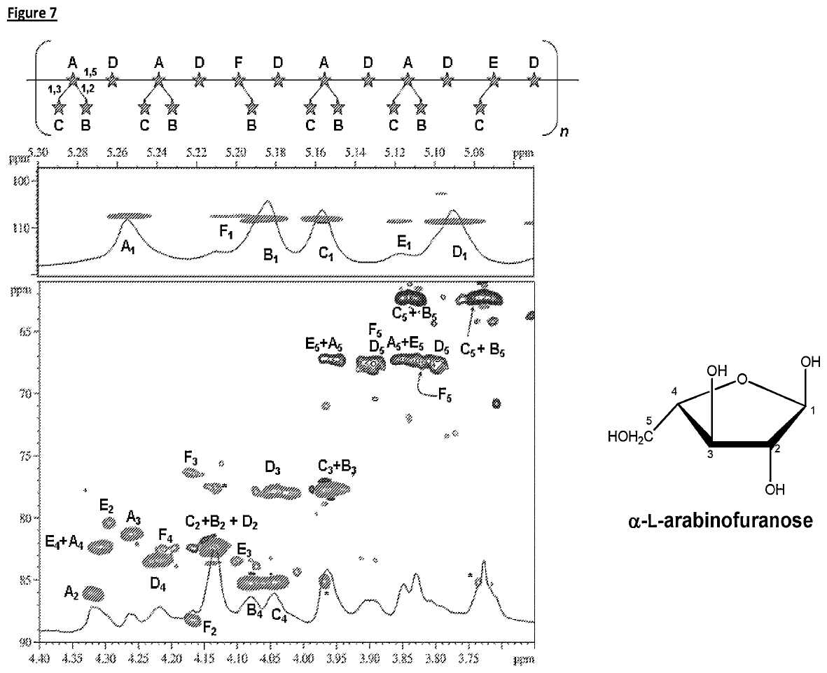

FIG. 7 (600 MHz, 298 K, D₂O) Expansion of the ¹H-¹³C HSQC spectrum of Br 18/F (pure arabinan with strong activity) detailing the (top) anomeric region, and (bottom) the carbinolic region along with the proton traces. The CH₂ groups (black) are distinguished from the other densities because the spectrum has been acquired in the DEPT mode. For the structure of the arabinan and for the labels used refer to FIG. 57B.

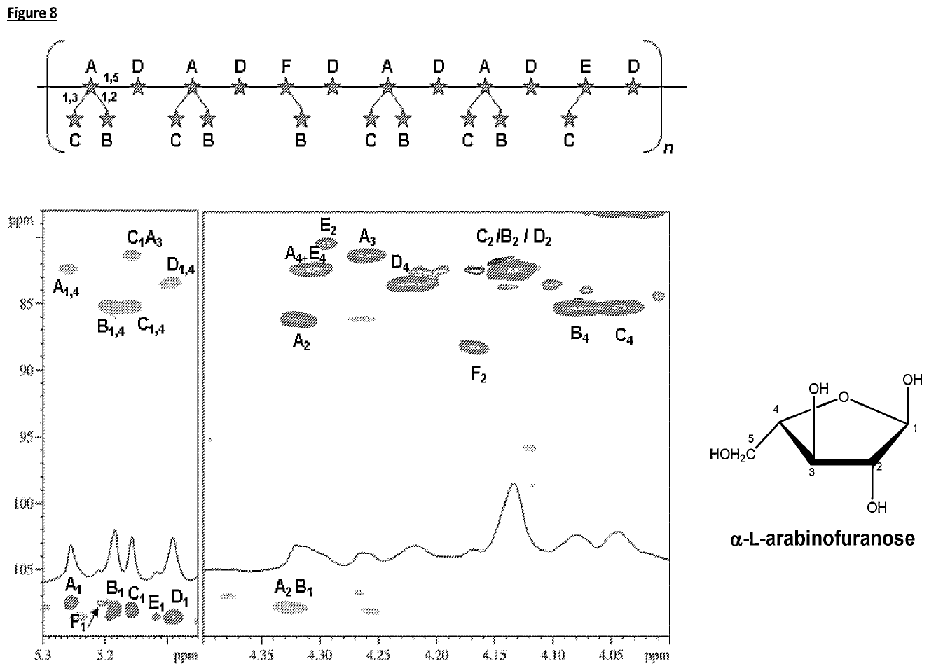

FIG. 8 (600 MHz, 298 K, D₂O) Selected regions of the NMR spectra reporting the overlap of the HSQC (dark grey) and HMBC (light grey) spectra of Br 18/F (pure arabinan with strong activity): anomeric (left panel) and carbinolic region (right panel). For the structure of the arabinan and for the labels used refer to FIG. 57B.

FIG. 9 (600 MHz, 298 K, D₂O) Expansion detailing the anomeric region of the arabinan (Br/18/F, the pure arabinan with strong activity) and reporting the overlap of the TOCSY (black) and COSY (dark and light grey) spectra. For the structure of the arabinan and for the labels used refer to FIG. 57B.

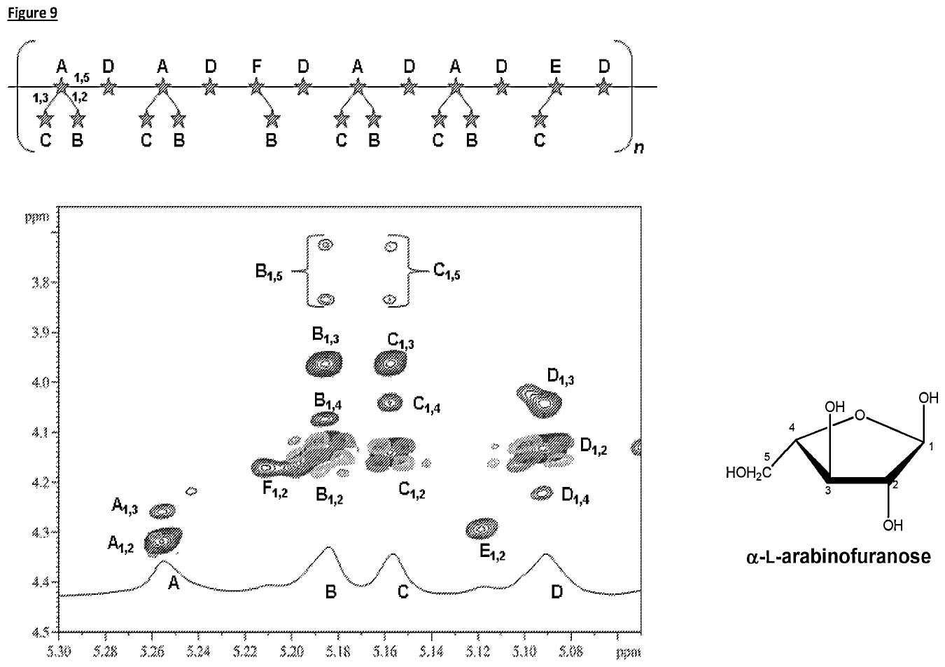

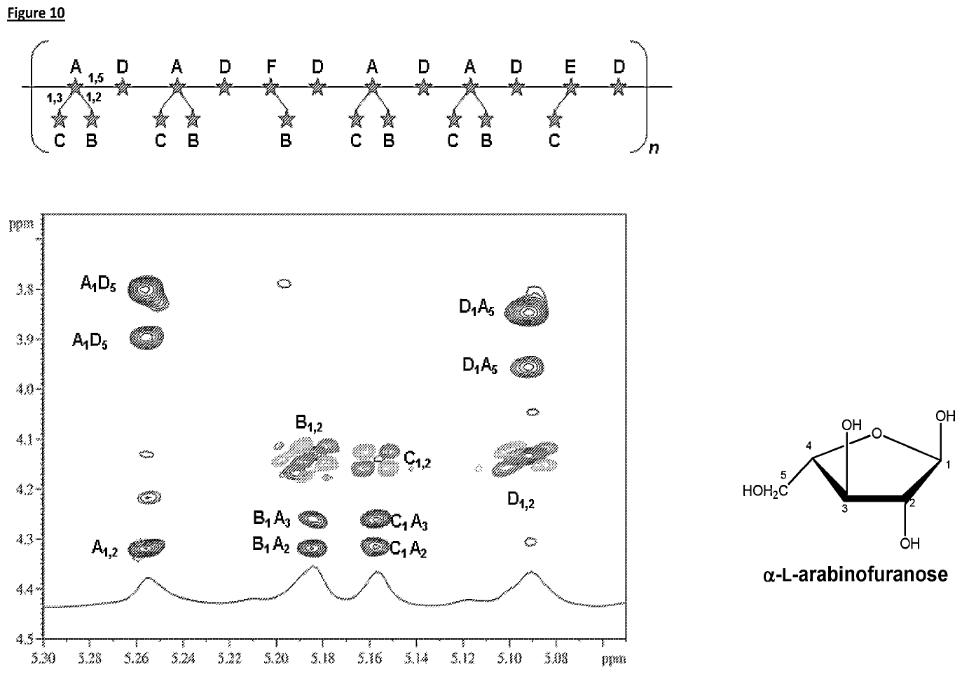

FIG. 10 (600 MHz, 298 K, D₂O) Expansion detailing the anomeric region of the arabinan (Br/18/F, the pure arabinan with strong activity) and reporting the overlap of the NOESY (black) and COSY (dark and light grey) spectra. For the structure of the arabinan and for the labels used refer to FIG. 57B.

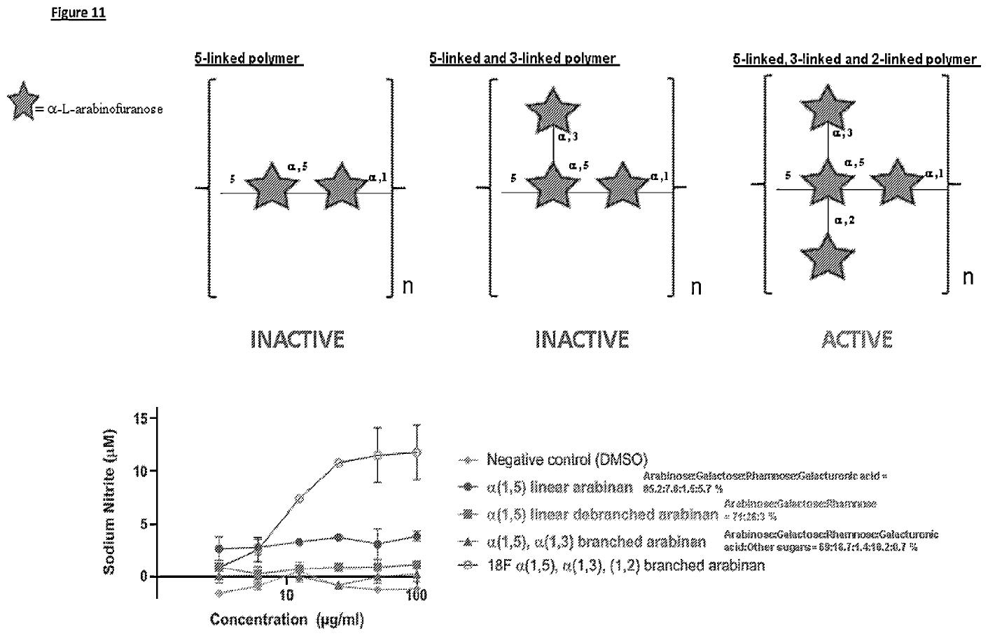

FIG. 11 shows the linkages important for immunomodulatory activity. Linear arabinan polysaccharides with only a 1-5 backbone are devoid of immunomodulatory activity. Arabinan polysaccharides with a 1-5 backbone and 1-3 branches are also devoid of activity. The only tested arabinan polysaccharide with immunomodulatory activity contains 1-5, 1-3 and also 1,2 linkages (18F). Measurements here are based on the production of NO within a macrophage cell line.

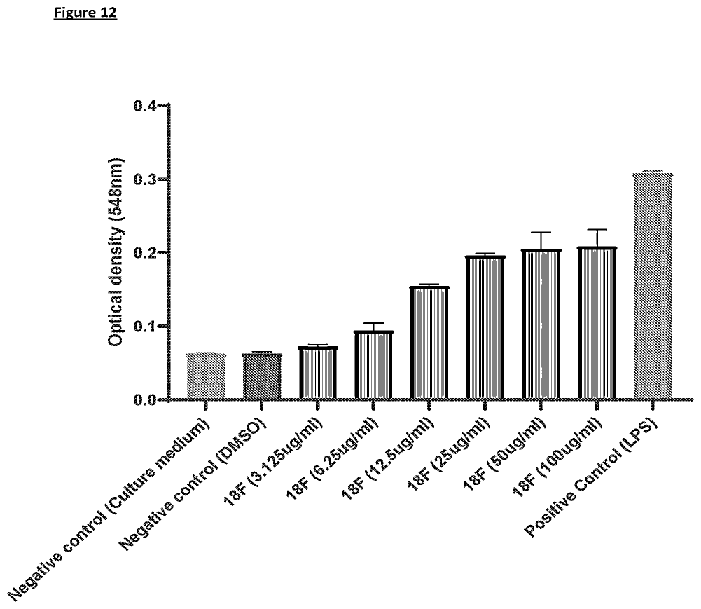

FIG. 12 shows the immunomodulatory activity of the 1-5, 1-3 and 1,2 linked arabinan polysaccharide (18F) is concentration dependent, significantly increasing NO production in a macrophage cell line as the added concentrations increase.

FIG. 13 shows that the molecular weight of purified amounts the 1-5, 1-3 and 1,2 linked arabinan polysaccharide may influence activity. Different preparations of the polysaccharide, all with the same 1-5, 1-3 and 1-2 structure, but differing in the molecular weight prepared can differ in the extent of their activity. A preparation containing polysaccharides with 66 KDa and 10 KDa molecular weights had strong activity (Br/36/A). A preparation containing polysaccharides with a 49 KDa molecular weight had moderate activity (Br/17/D). A preparation containing polysaccharides with a 12 KDa molecular weight also had moderate activity (18F). Finally, a preparation containing polysaccharides with a 3 Da molecular weight had poor/negligible activity (Br34F).

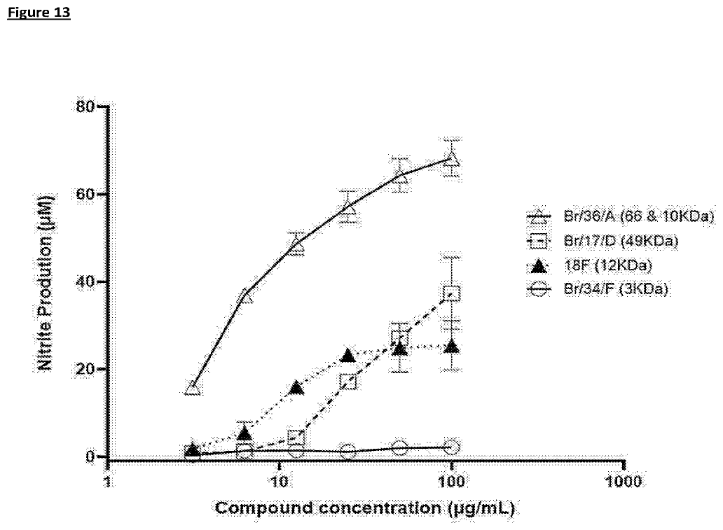

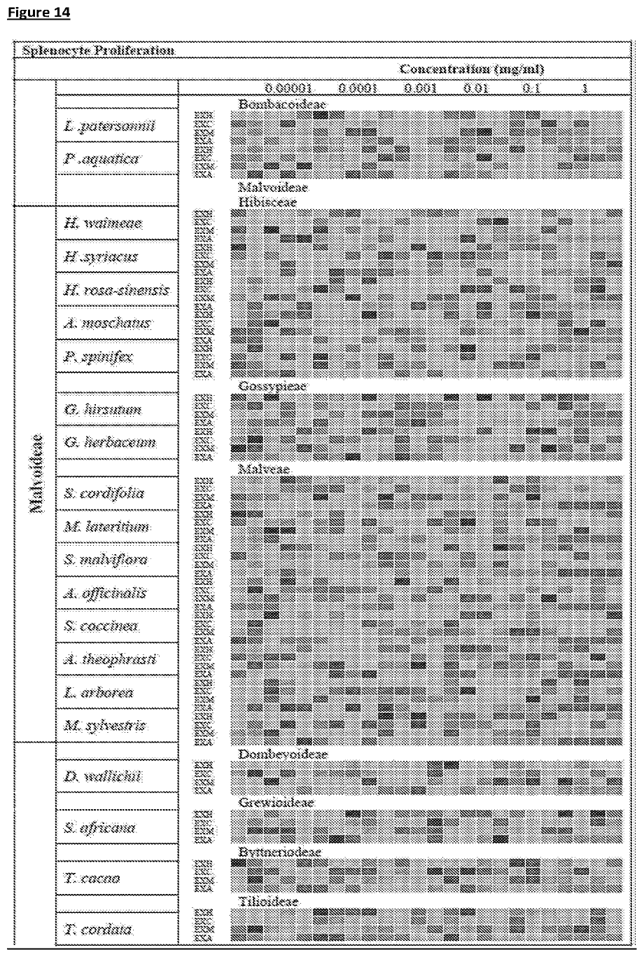

FIG. 14 is a heat map showing Splenocyte proliferation EXH hexane extract; EXC chloroform extract; EXM methanol extract; EXA aqueous extract. Red>Yellow>Blue.

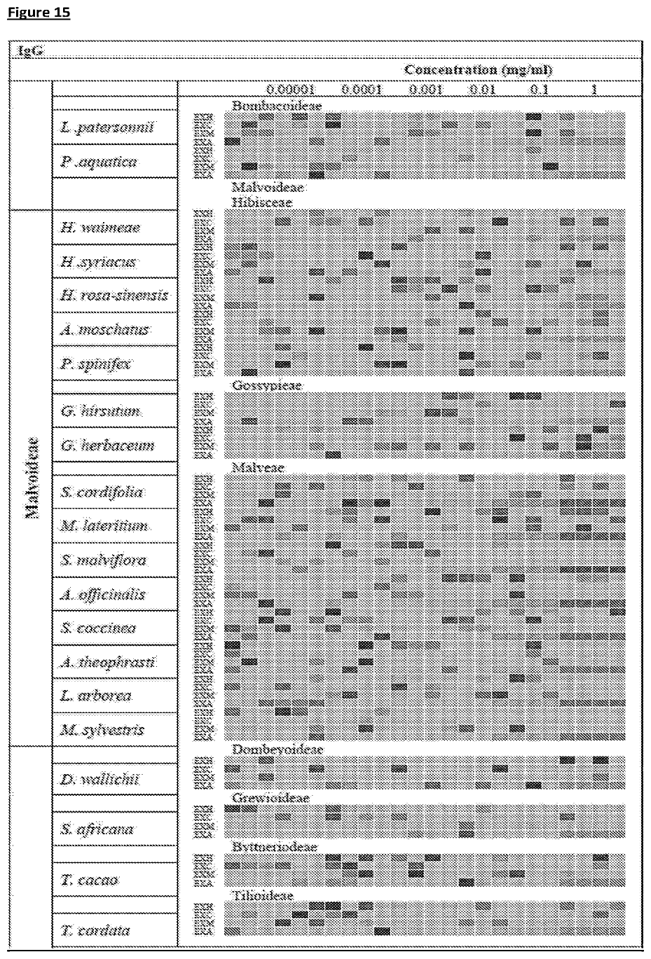

FIG. 15 is a heat map showing antibody secretion (IgG) EXH hexane extract; EXC chloroform extract; EXM methanol extract; EXA aqueous extract. Red>Yellow>Blue.

FIG. 16 is a heat map showing antibody secretion (IgA) EXH hexane extract; EXC chloroform extract; EXM methanol extract EXA aqueous extract. Red>Yellow>Blue.

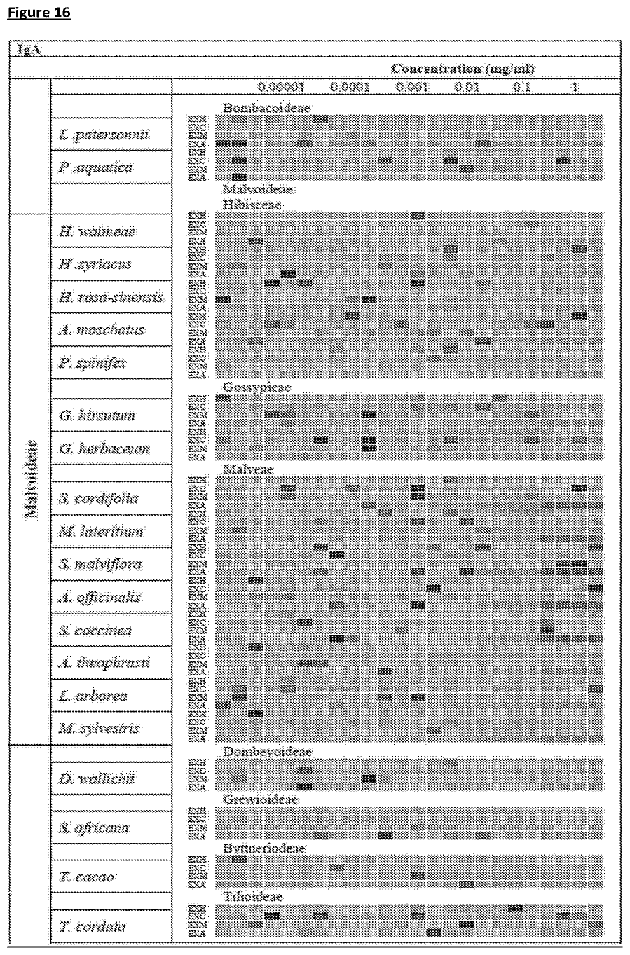

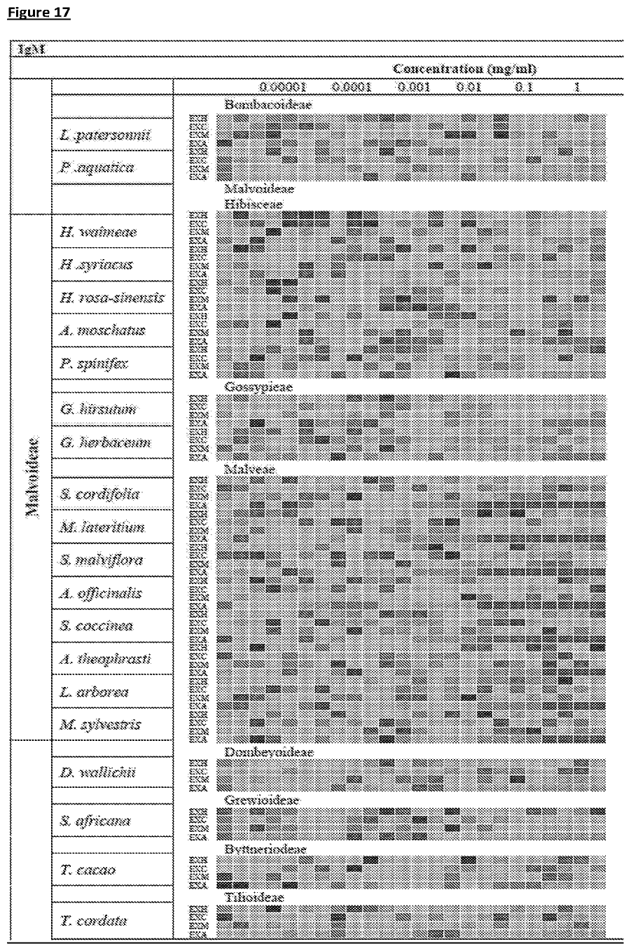

FIG. 17 is a heat map showing antibody secretion (IgM) EXH hexane extract; EXC chloroform extract; EXM methanol extract EXA aqueous extract. Red>Yellow>Blue.

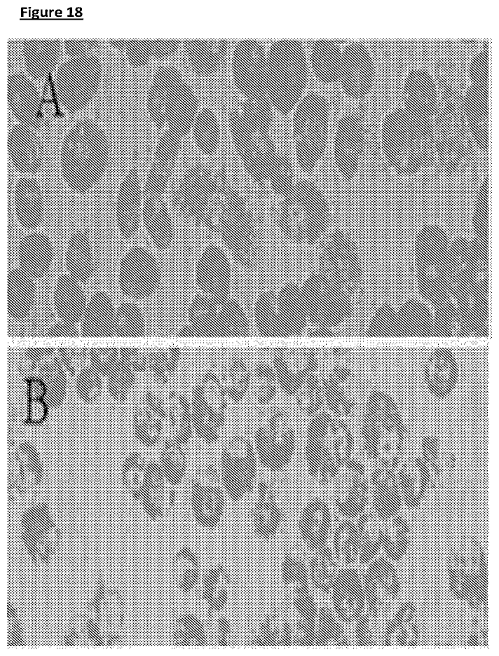

FIG. 18 shows RAW 264.7 macrophages. (A) depicts macrophages that had been exposed to aqueous (crude) extracts of Sida cordifolia, and subsequently incubated in media containing neural red. On exposure to aqueous extracts of the malveae the cells appear far larger in size, with an increased ability of endocytosis, hence a more intense red colour in cell cytoplasm. (B) shows macrophages which were treated in same conditions but not exposed to polysaccharides, they appeared smaller with evidently less neutral red ingested.

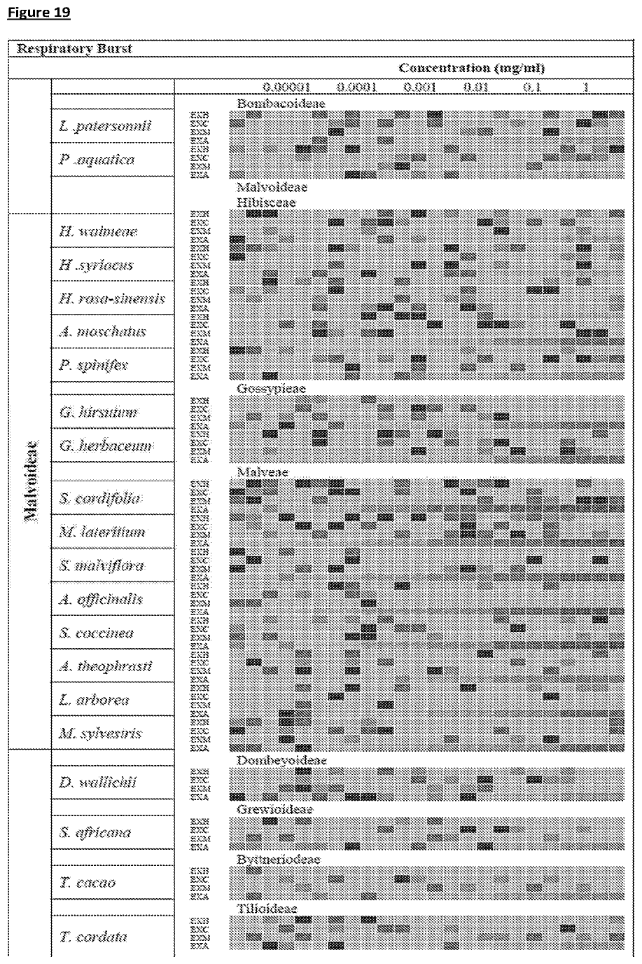

FIG. 19 is a heat map showing effects of extracts on respiratory burst EXH hexane extract; EXC chloroform extract; EXM methanol extract EXA aqueous extract. Red>Yellow>Blue.

FIG. 20 is a heat map showing phagochagocytosis rate EXH hexane extract; EXC chloroform extract; EXM methanol extract EXA aqueous extract. Red>Yellow>Blue.

FIG. 21 shows the results of (A) a splenocyte proliferation assay, with extracts of Sidalcea malviflora as an example (B) IgA secretion with S. malviflora (C) IgG production with S. malviflora (D) IgM Production with S. malviflora.

FIG. 22 shows the results of a splenocyte proliferation assay with S. malviflora extracts with the ELISA rdU assay.

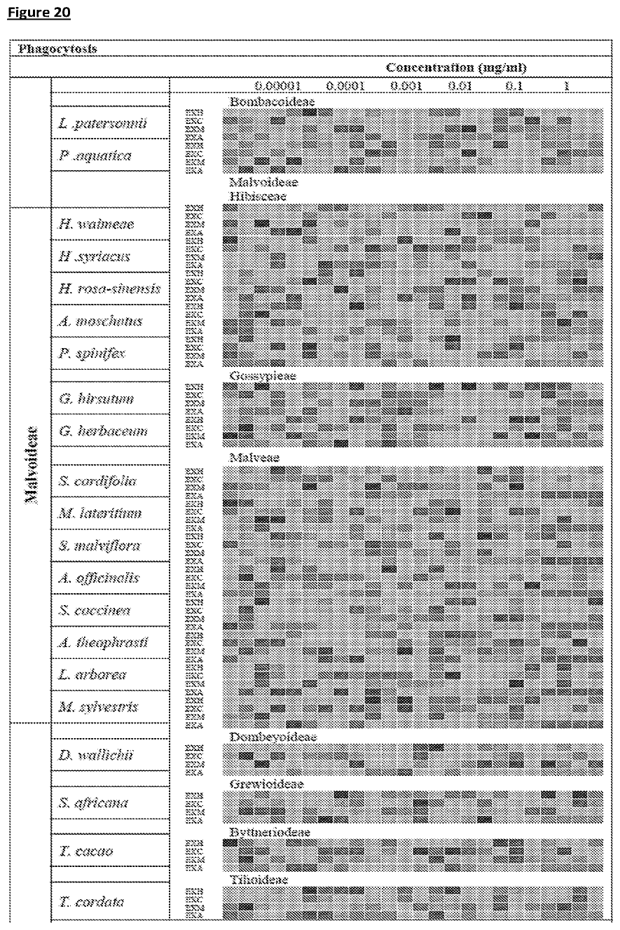

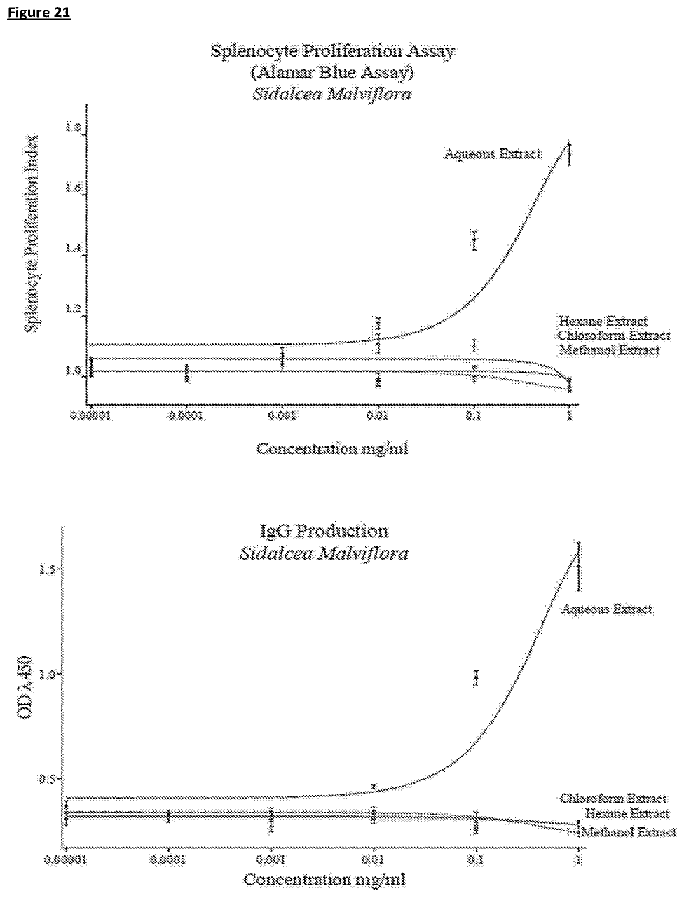

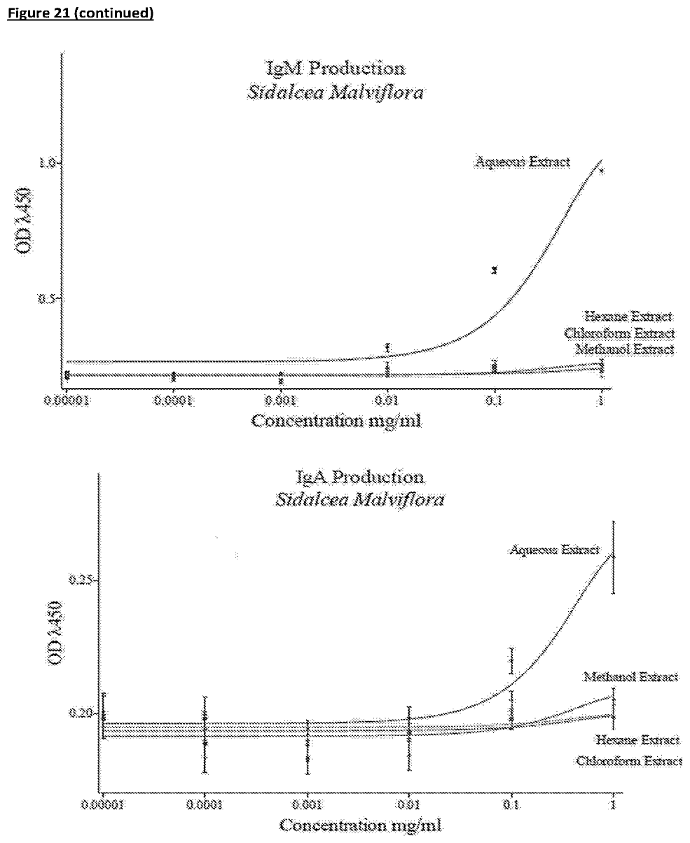

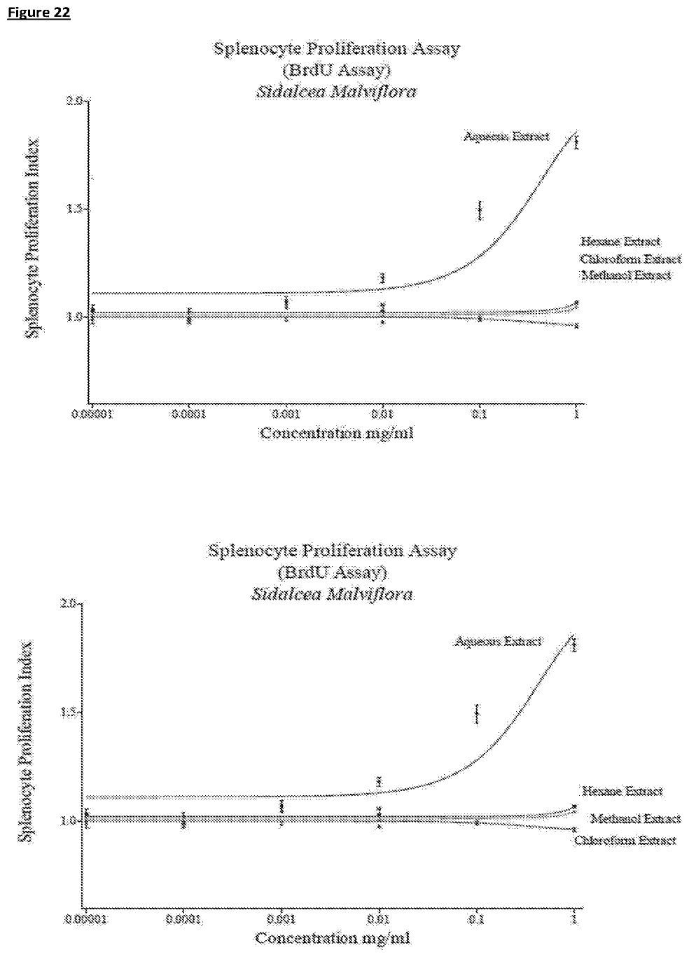

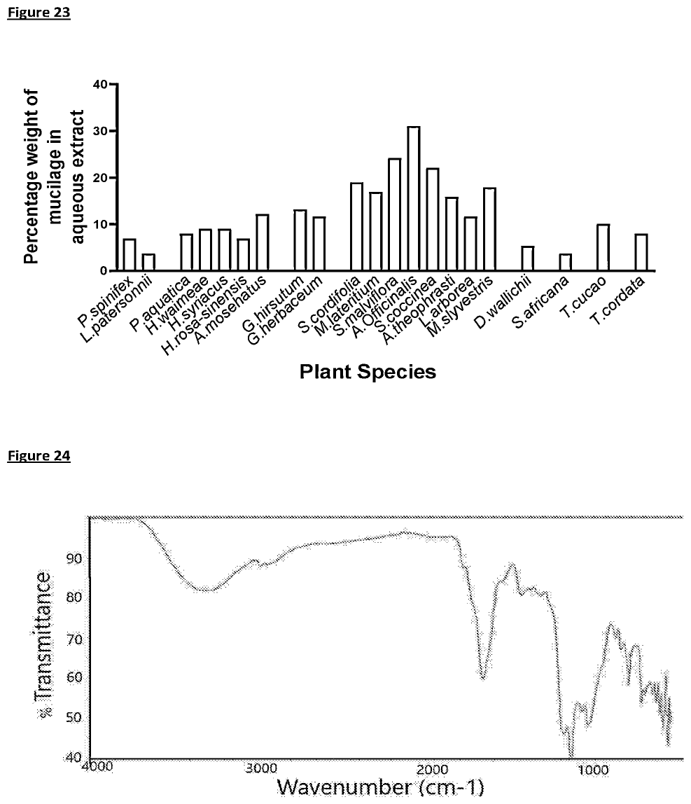

FIG. 23 is a bar chart showing the % wt. mucilage in aqueous extracts of malveae.

FIG. 24 show an IR spectra produced with polysaccharide precipitate M. sylvestris.

FIG. 25 is a chart depicting percentage of monosaccahrides constituting polysaccharides of malveae (A) Sidalceae malviflora (malveae sub malvoideae) (B) Malva sylvesteris (malveae sub malvoideae).

FIG. 26 shows charts depicting percentage of monosaccahrides constituting polysaccharides of malveae (A) Malva sylvestris (malveae sub malvoideae) (B) Sphaeralcea coccinea (malveae sub malvoideae).

FIG. 27 shows charts depicting percentage of monosaccahrides constituting polysaccharides among subfamilies of malvacea in which immunomodulatory effects were not observed (A) Lagunaria patersonii (subfamily; Bombacoideae) (B) Hibiscus rosa-sinensis (subfamily; malvoideae tribe; Hibisceae).

FIG. 28 is a heat map comparing the splenocyte proliferation observed in both aqueous extracts (EXA) and the aqueous extract preicipitate (EXAP) which was produced from the aqueous extract by ethanol precipitation.

FIG. 29 is a heat map comparing the lymphocyte IgG production in both aqueous extracts (EXA) and the aqueous extract precipitate (EXAP), produced from the aqueous extract by ethanol precipitation.

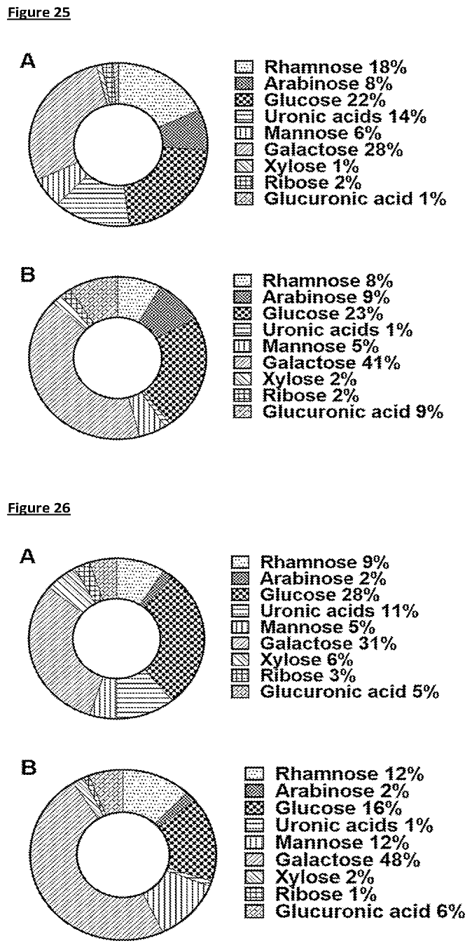

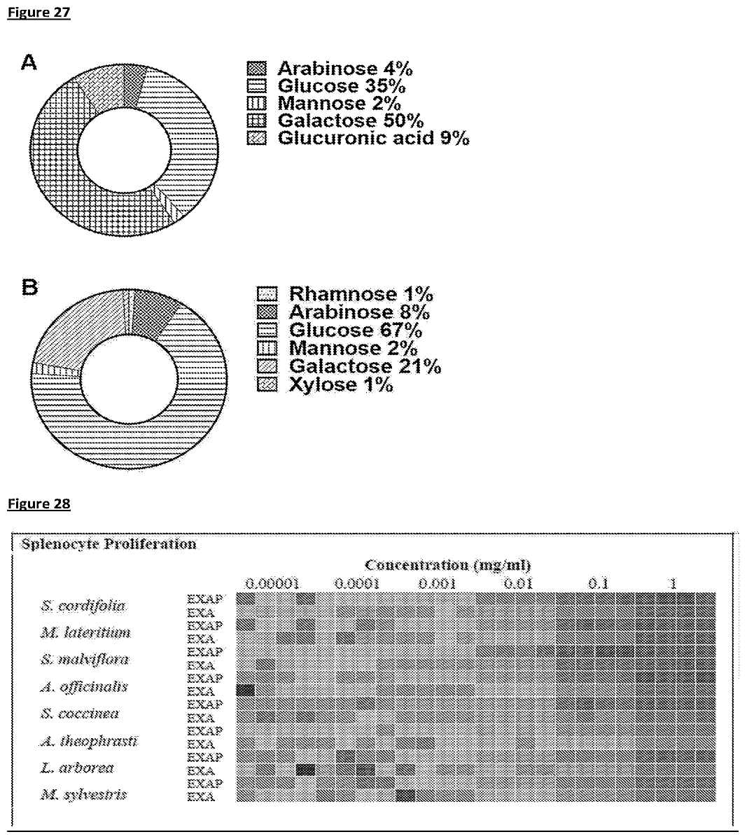

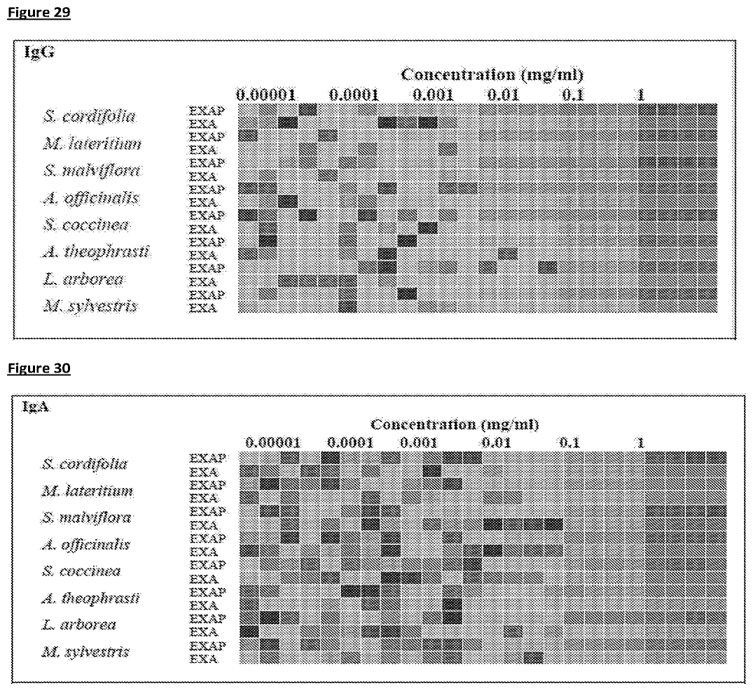

FIG. 30 is a heat map comparing the lymphocyte IgA production in both aqueous extracts (EXA) and the aqueous extract precipitate (EXAP), produced from the aqueous extract by ethanol precipitation.

FIG. 31 is a heat map comparing the lymphocyte IgM production in both aqueous extracts (EXA) and the aqueous extract precipitate (EXAP), produced from the aqueous extract by ethanol precipitation.

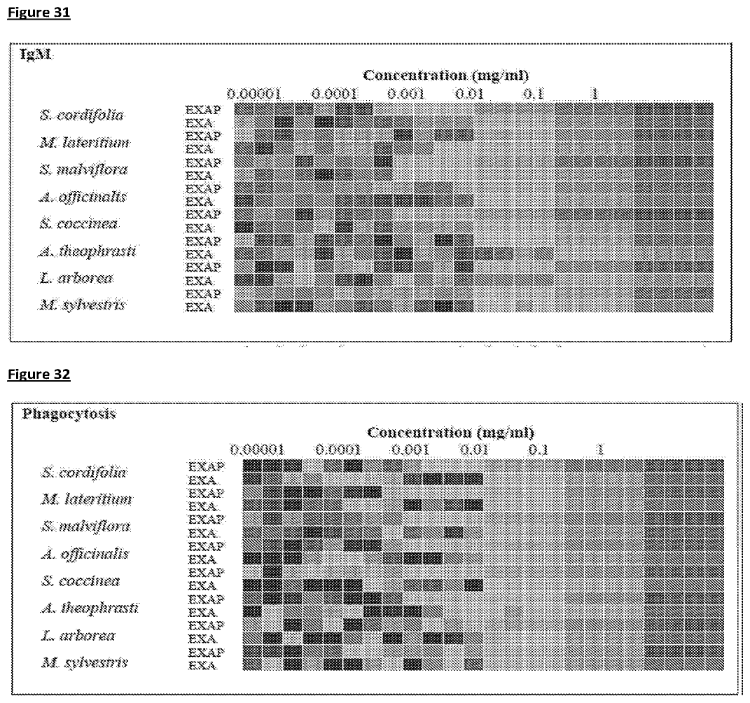

FIG. 32 is a heat map comparing the macrophage phagocytic activities of both the aqueous extracts (EXA) and the aqueous extract precipitate (EXAP) produced from the aqueous extract by ethanol precipitation.

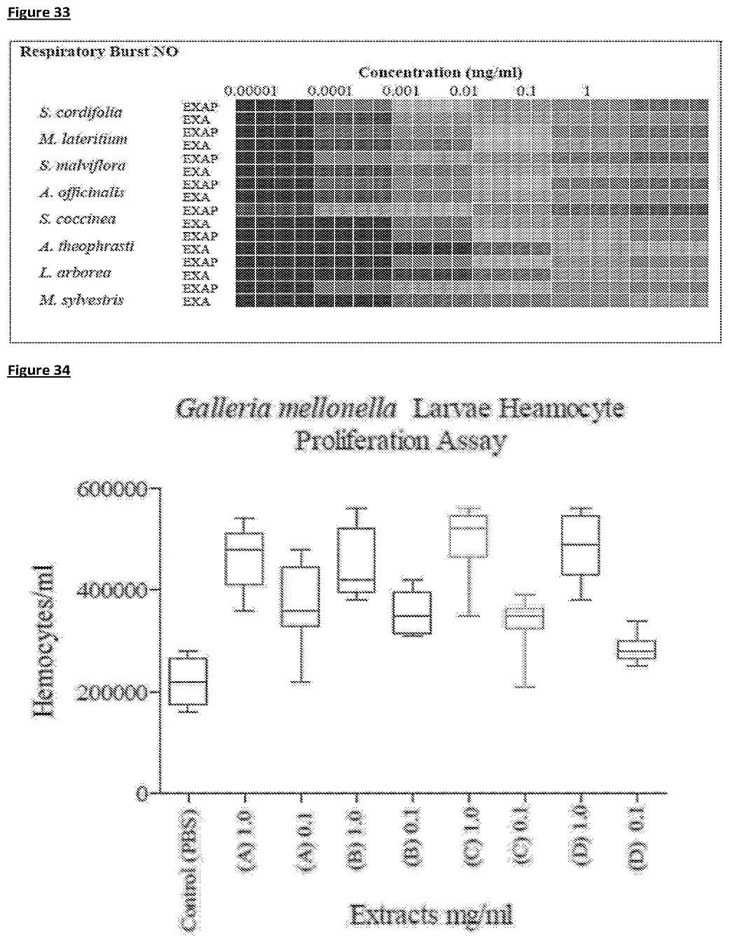

FIG. 33 is a heat map comparing the macrophage respiratory burst of the aqueous extracts (EXA) and the aqueous extract precipitate (EXAP) produced from the aqueous extract by ethanol precipitation.

FIG. 34 shows the haemocyte count per ml of haemolymph of Galleria larvae. (A) S. cordiflia (B) M. sylvesteris (C) M. lateritium (D) S. malviflora.

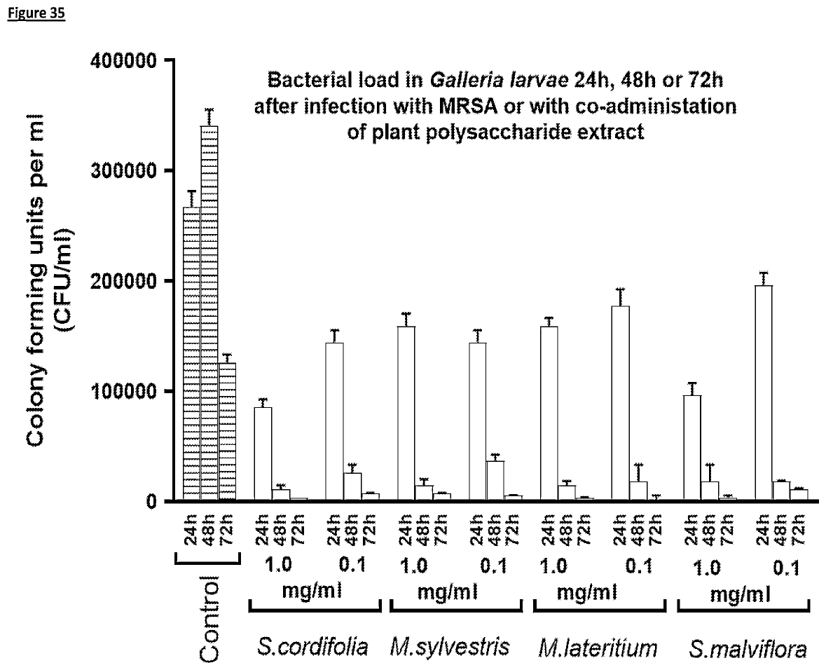

FIG. 35 shows the CFU/ml in larvae pretreated with polysaccharide enriched aqueous extract (A) S. cordiflia (B) M. sylvesteris (C) M. lateritium (D) S. malviflora.

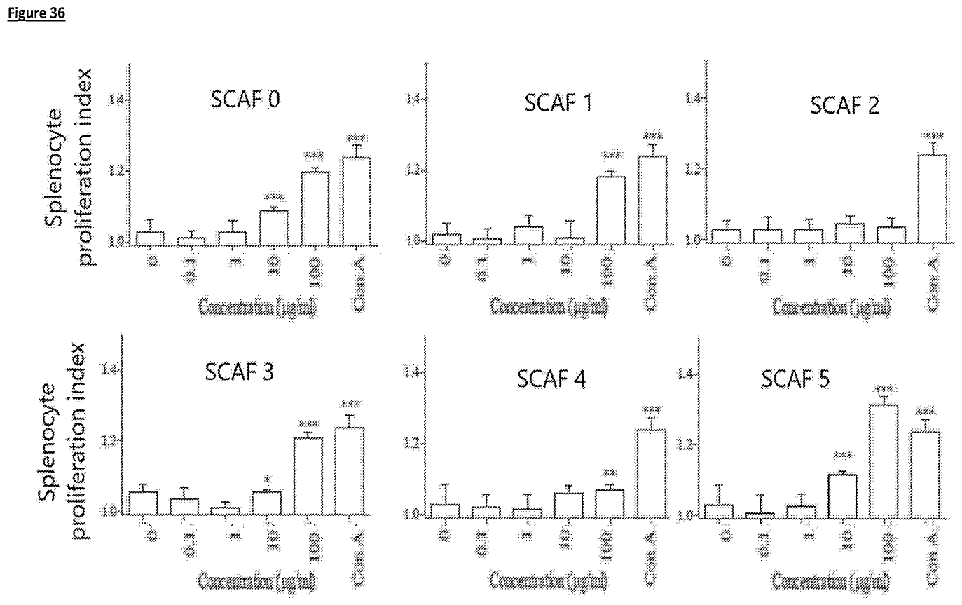

FIG. 36 shows the results of splenocyte proliferation assays. Groups are SCAF 0 (crude polysaccharide fraction of S. cordifolia); SCAF 1: Low ionic strength <100 kDa; SCAF 2: medium ionic strength and between 10-100 kDa; SCAF 3: medium ionic strength and >100 kDa; SCAF 4 high ionic strength and between 10 kDa-100 kDa; SCAF 5: high ionic strength and <100 kDa; and CON A (positive control).

FIG. 37 shows the IgG secretions by splenocytes incubated with extracts of Sida cordofilia. Groups are SCAF 0 (crude polysaccharide fraction of S. cordifolia); SCAF 1: Low ionic strength <100 kDa; SCAF 2: medium ionic strength and between 10-100 kDa; SCAF 3: medium ionic strength and >100 kDa; SCAF 4 high ionic strength and between 10 kDa-100 kDa; SCAF 5: high ionic strength and <100 kDa; and CON A (positive control).

FIG. 38 shows the IgM secretions by splenocytes incubated with extracts of Sida cordofilia. Groups are SCAF 0 (crude polysaccharide fraction of S. cordifolia); SCAF 1: Low ionic strength <100 kDa; SCAF 2: medium ionic strength and between 10-100 kDa; SCAF 3: medium ionic strength and >100 kDa; SCAF 4 high ionic strength and between 10 kDa-100 kDa; SCAF 5: high ionic strength and <100 kDa; and CON A (positive control).

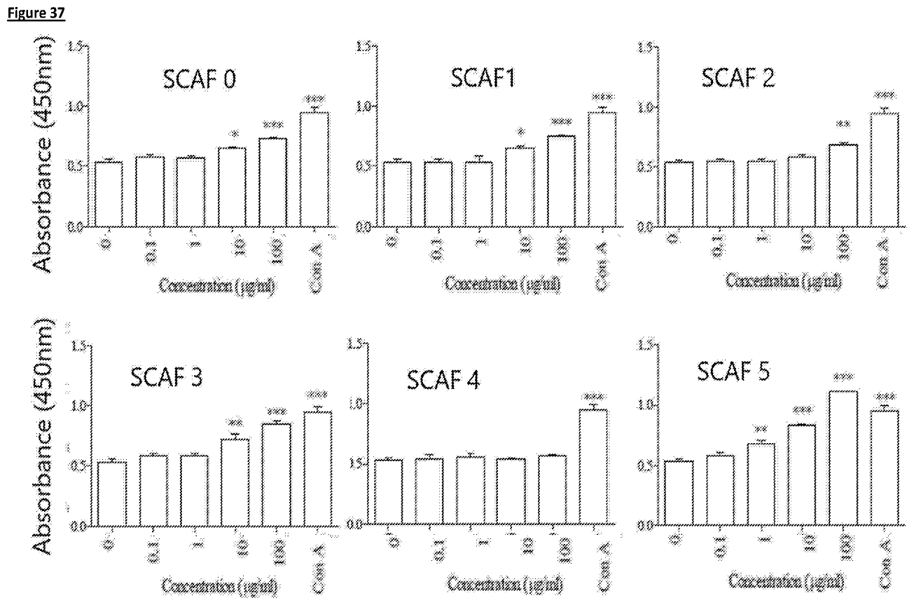

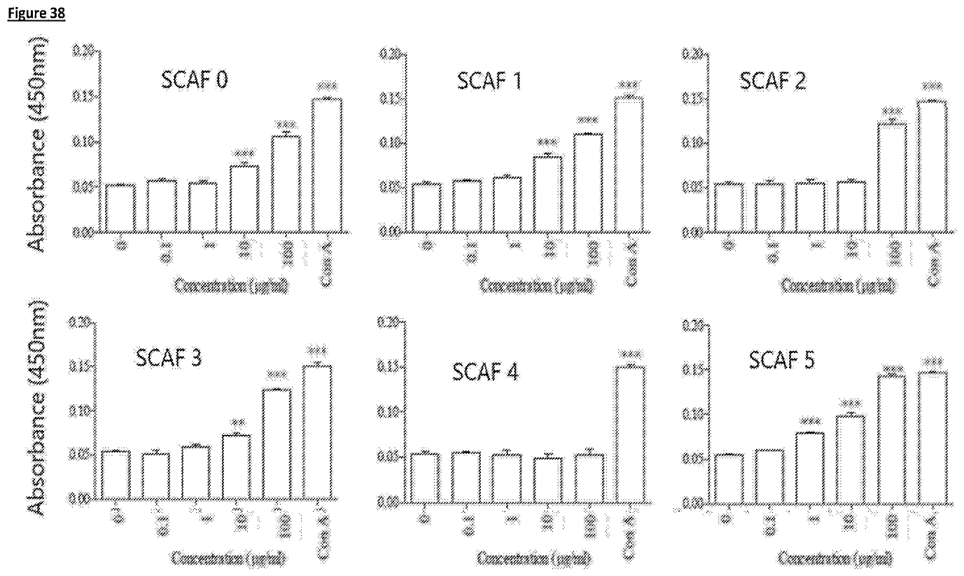

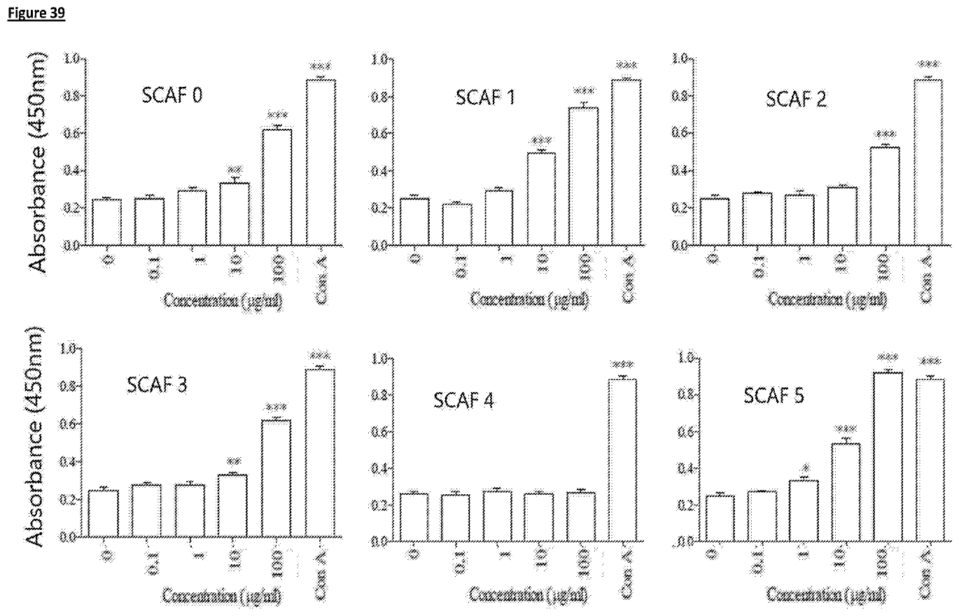

FIG. 39 shows the IgA secretions by splenocytes incubated with extracts of Sida cordofilia. Groups are SCAF 0 (crude polysaccharide fraction of S. cordifolia); SCAF 1: Low ionic strength <100 kDa; SCAF 2: medium ionic strength and between 10-100 kDa; SCAF 3: medium ionic strength and >100 kDa; SCAF 4 high ionic strength and between 10 kDa-100 kDa; SCAF 5: high ionic strength and <100 kDa; and CON A (positive control).

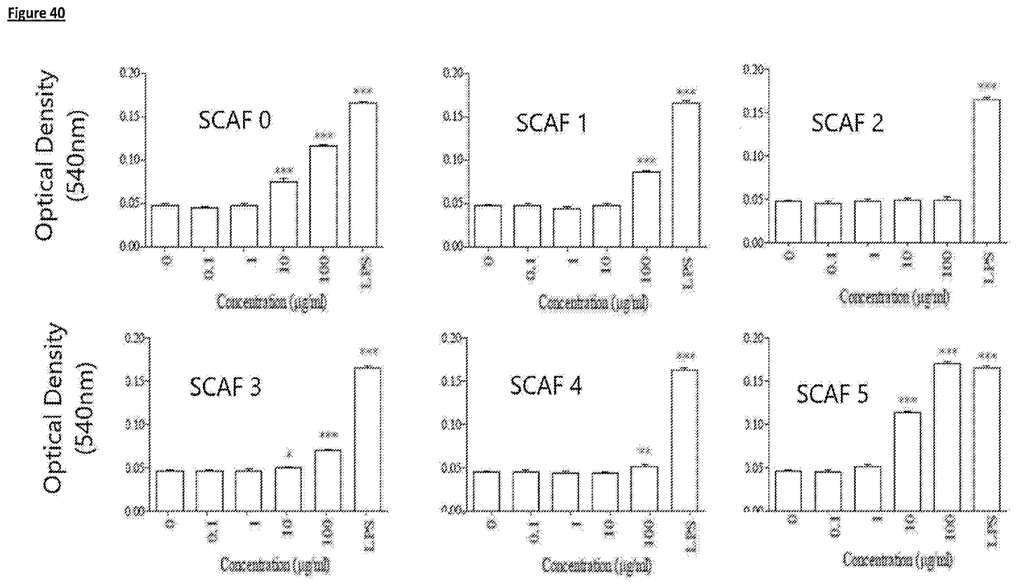

FIG. 40 show the results of a neutral red phagocytosis assay. Groups are SCAF 0 (crude polysaccharide fraction of S. cordifolia); SCAF 1: Low ionic strength <100 kDa; SCAF 2: medium ionic strength and between 10-100 kDa; SCAF 3: medium ionic strength and >100 kDa; SCAF 4 high ionic strength and between 10 kDa-100 kDa; SCAF 5: high ionic strength and <100 kDa; and LPS (positive control).

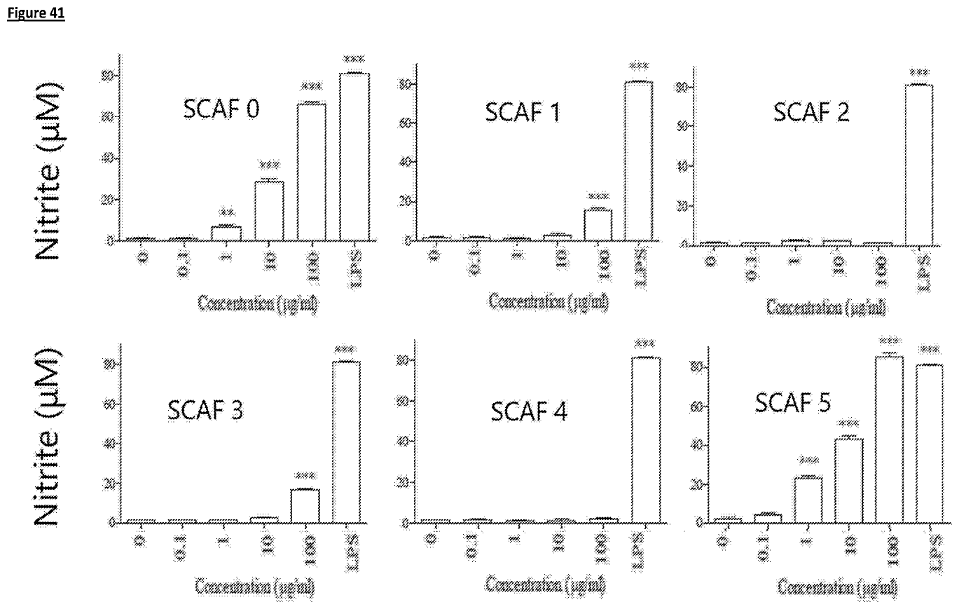

FIG. 41 shows the nitrite concentration detected in growth medium (RAW264 cells) incubated with extracts of Sida cordofilia.

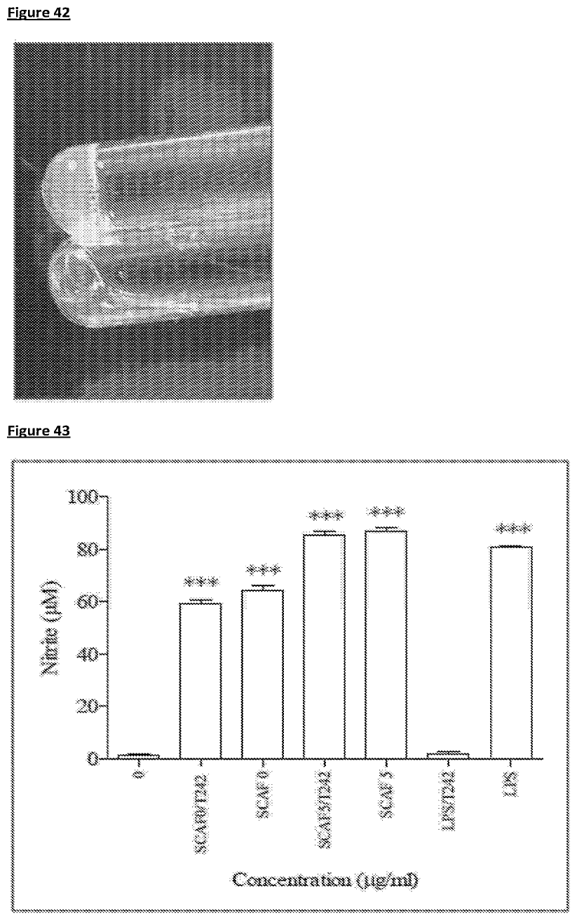

FIG. 42 shows a tube used to perform a Limulus amebocyte lysate (LAL) assay. LAL is an aqueous extract of blood cells from the horse-shoe crab which coagulates on detecting endotoxins. The lower tube contains the 2SCAF 5 and the upper tube containing LPS endotoxin. Coagulation of the upper confirms presence of endotoxin. However, the failure to coagulate blood upon adding SCAF 1-5 verified the absence of endotoxins.

FIG. 43 shows the results of a TLR4 inhibition assay using TAK-242. The effect of extracts and TAK-242 on nitrite production by macrophages was monitored. The failure to inhibit activation of macrophages when cells are incubated with TAK-242 suggests the activation pathway does not involve the TL4 receptor.

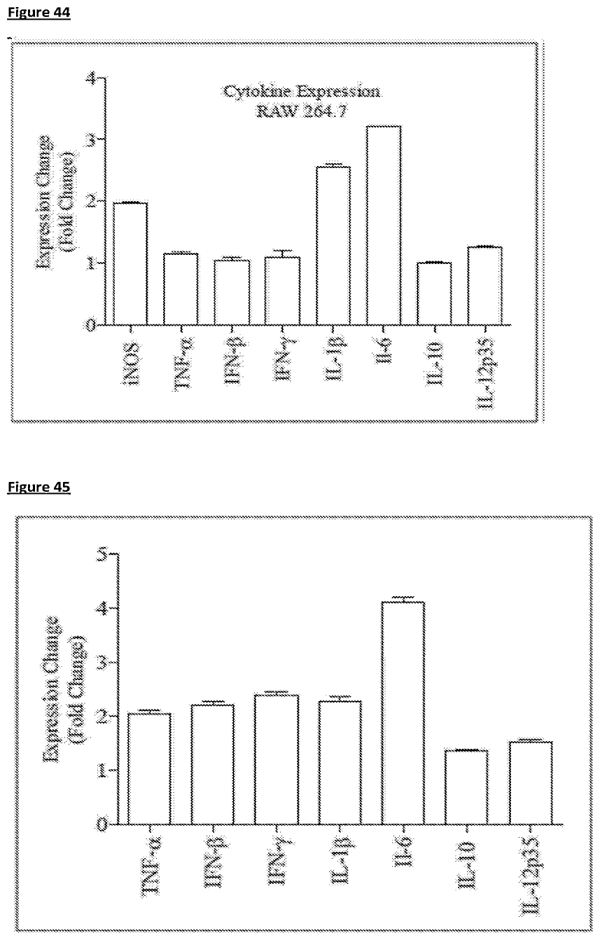

FIG. 44 shows cytokine expression by RAW 264.7 following exposure to SCAF 5.

FIG. 45 shows cytokine expression by splenocytes following exposure to SCAF 5.

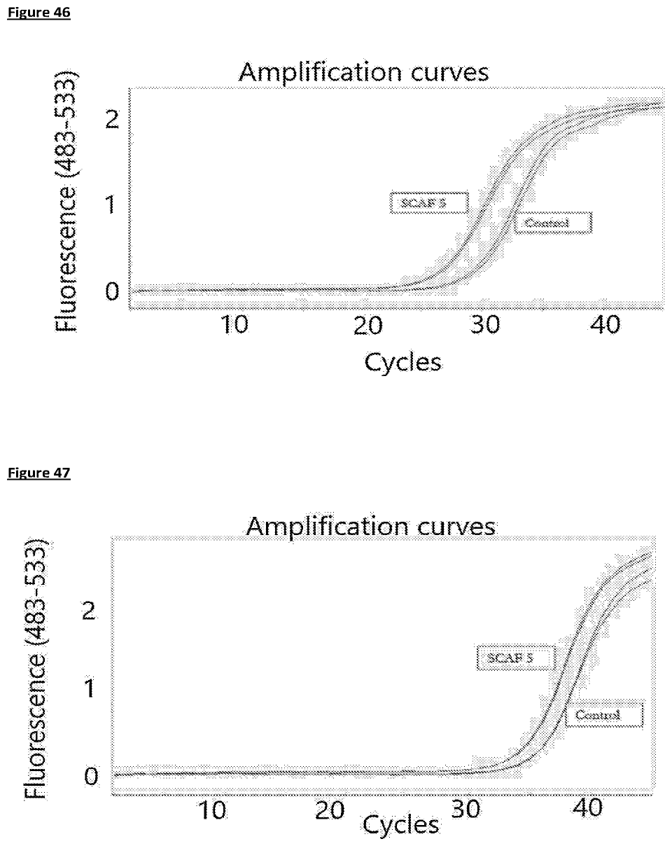

FIG. 46 shows an amplification curve of iNOS following exposure to SCAF 5 (RAW 264.7). Lines show untreated control versus treatment with SCAF 5.

FIG. 47 shows an amplification curve for IL-6 following exposure to SCAF 5 (splenocytes). Lines show untreated control versus treatment with SCAF 5.

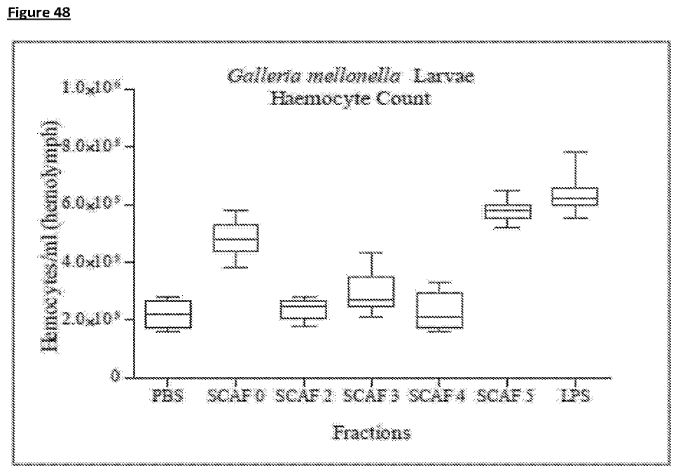

FIG. 48 shows haemocyte numbers of Galleria mellonella larvae exposed to S. cordofilia. Haemocytes enumeration per ml of haemolymph post administration (24 h; SCAF1-SCAF5; 100 μg/ml). LPS (1 μg/ml).

FIG. 49 shows bacterial load (ACTCC 43300) 24 h post-infection in Galleria larvae exposed to SCAF 0 and SCAF5 (20 μl, 100 μg/ml). Control larvae (20 μl PBS).

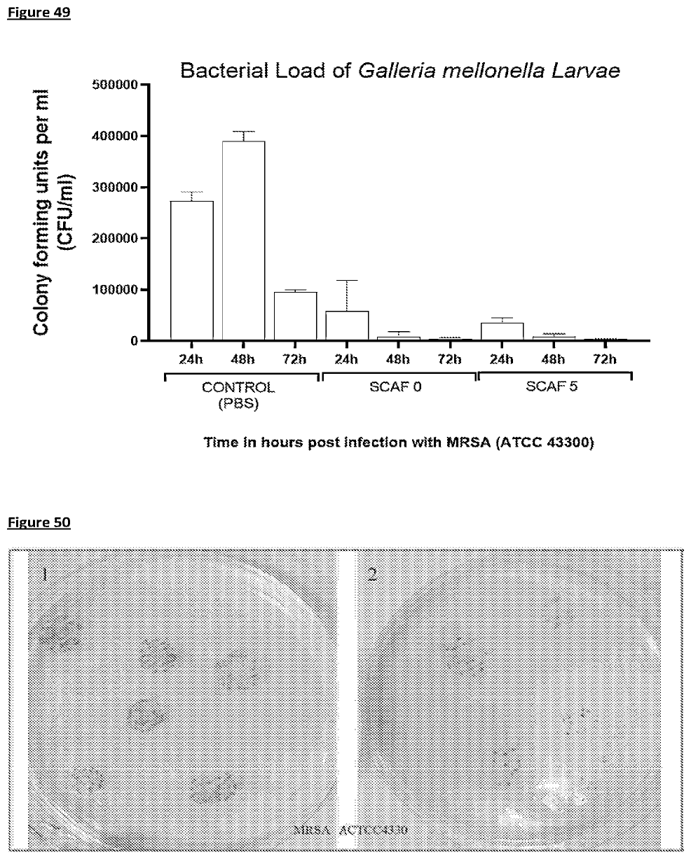

FIG. 50 shows (1) the bacterial load (MRSA 43300) in haemolymph of galleria larvae 24 h after treatment with PBS, colonies stained with MTT. (2) the bacterial load (MRSA 43300) in haemolymph of Galleria larvae 24 h aftertreatment with SCAF 5 (20 μl; 100 μg/ml). Colonies were visualised with MTT.

FIG. 51 is a concentration-effect curve in RAW 264.7 macrophages treated with the polysaccharide according to the invention. The measured effect is NO production

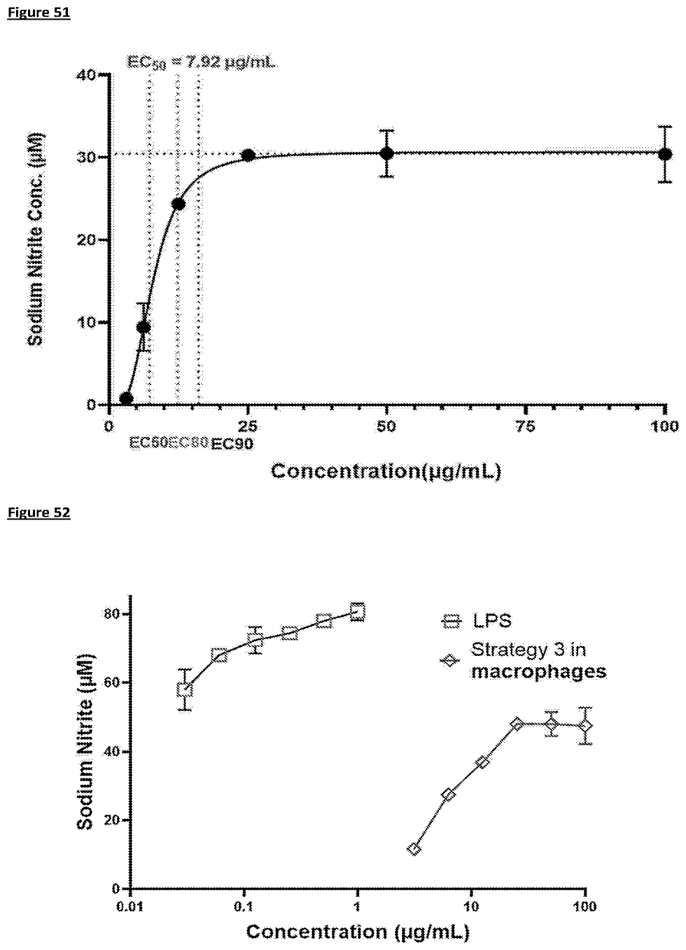

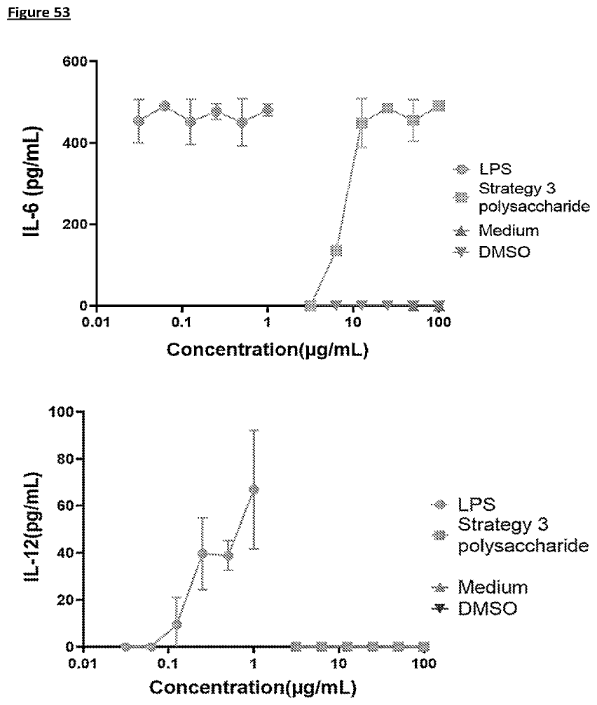

FIG. 52 is a concentration-effect curve in RAW 264.7 macrophages treated with the polysaccharide according to the invention or LPS. The measured effect is NO production.

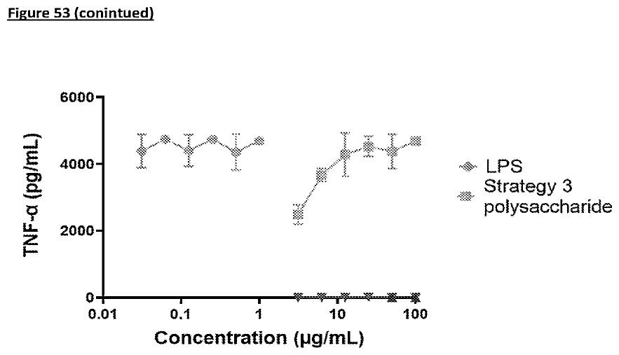

FIG. 53 shows the effect of the polysaccharide according to the invention on cytokine production by macrophages.

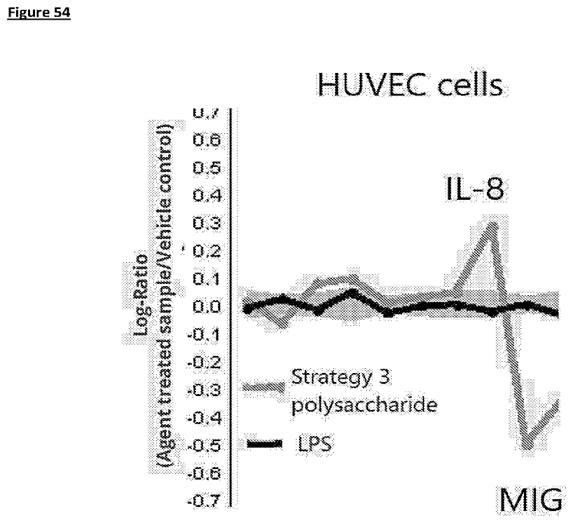

FIG. 54 shows the effect of the polysaccharide according to the invention on cytokine production by human umbilical vein endothelial cells.

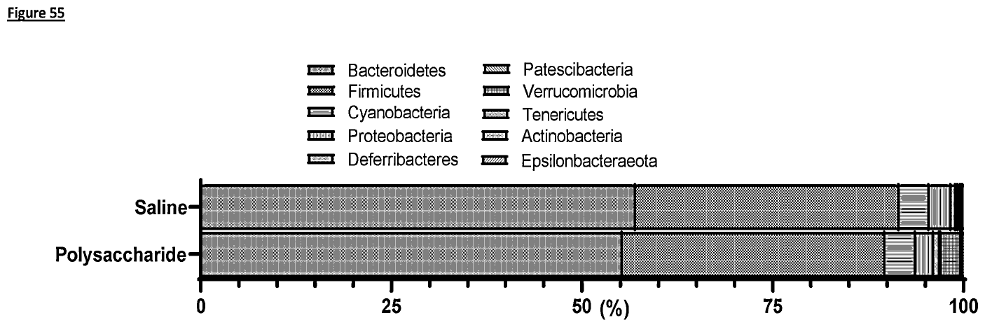

FIG. 55 shows, at the phyla level, the effect of an isolated polysaccharide according to the invention on the faecal bacterial composition of C57Bl/6J mice.

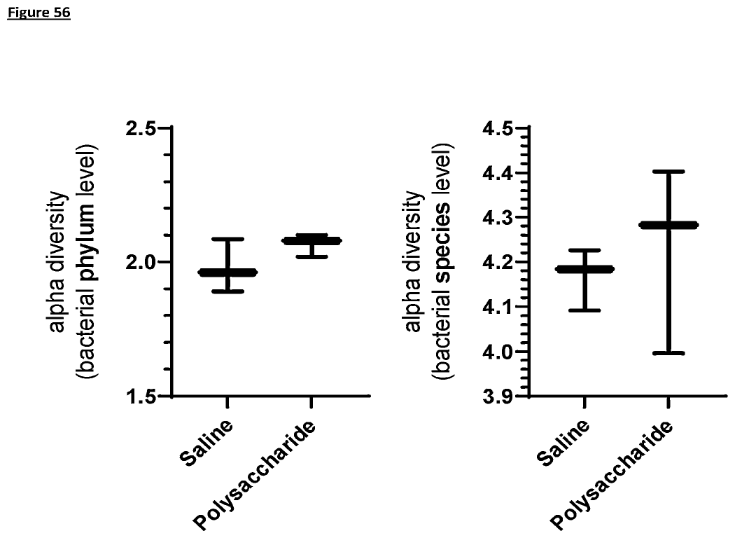

FIG. 56 shows the effect of an isolated polysaccharide according to the invention on the faecal bacterial diversity of C57Bl/6J mice at the phyla and species level.

### EXAMPLES

### Example 1—Isolation of the Plant Polysaccharide

The active arabinan polysaccharide of the invention can be isolated from plant material in a number of ways. An optimised 3-step extraction process (outlined in FIG. 1) leads to active arabinan residing in Br/29/16 (Br/29/17 is inactive). The active arabinan is present and highly enriched in Br/29/16 and is relatively pure but may be further purified by chromatographic methods.

Step I—initial extraction takes place with an overnight incubation of powered plant material in 95% ethanol at room temperature. Extraction is completed by centrifugation to obtain the precipitate pellet at the bottom of the tube. The remaining ethanol phase (Br/29/2) is discarded.

Step II—further extraction takes place by 6 repeated cycles of adding water to the pellet, boiling this (1 h; 100° C.) using an oil bath and a thermocouple, then centrifugation (see details in FIG. 1). Each time the water phases (e.g. Br/29/3, 5, 7, 9, 11 and 14) are combined together, and the pellet (e.g. Br/29/4, 6, 8, 10 and 12) at the bottom of the tube undergoes a further extraction cycle.

Step III—the pooled extract is reduced in volume by a rotary evaporator. This undergoes enzymatic digestion (37° C.) to remove any unwanted polysaccharides and proteins (FIG. 1). The added enzymes include pullulanase (EC 3.2.1.41; Sigma Aldrich); α-Amylase (EC 3.2.1.1; Sigma Aldrich) and Protease k (EC 3.4.21.14; Sigma Aldrich). This digestate is then dialysed (Spectra/Por®4 Dialysis Membrane Standard RC Tubing; MWCO: 12-14 KDa; Thermo Fisher), and the final polysaccharide preparation (Br/29/16) is obtained by precipitation with ethanol and by a final centrifugation step. This pellet has potent immunomodulatory activity.

### Example 2—Determining the Chemical Structure of the Plant Polysaccharide in the Active Fraction of Sida cordofolia, and Optimisation of the Extraction/Purification Methodology

An extract from the roots of Sida cordofolia was purified by gel-filtration, HPLC and other chromatography methods specified. A process of bioactivity-guided fractionation was undertaken to identify highly purified polysaccharides fractions with activity. Several different extraction/purifications strategies were undertaken and the resulting polysaccharides were evaluated in terms of: (i) activity, (ii) yield, (iii) purity, and (iv) molecular weight. Activity was determined by measuring NO responses in a macrophage cell line as previously described. Yield was determined by comparing dried weight of fractions against initial dry weight. Purity was assessed by NMR or chromatography as appropriated. The molecular weight was determined by SEC-HPLC (TSK gel G5000 PWXL, 30 cm×7.8 mm ID). The purified arabinan polysaccharides (30 μl; 1 mg/ml solution) were injected on the TSK column and eluted with 100% of 50 mM ammonium bicarbonate (Flow 0.8 ml/min). The eluate was monitored by refractive index. The column was calibrated with dextrans of known molecular weight (5 KDa, 50 KDa, 150 KDa, 410 KDa, 610 KDa) and the data fitted in a linear regression and the molecular weight evaluated accordingly (FIG. 2). The purified arabinan polysaccharide with strong immunomodulatory activity underwent:
quali-quantitative analysis of the monosaccharide components by CG-MS;
site of linkage analysis of the monosaccharide components (see FIG. 3); and
1D and 2D NMR execution and analysis of the pure polymer (see FIGS. 4-10).

TABLE 2
Structure of the labelled residues.

| Label | Residue |
|---|---|
| A | 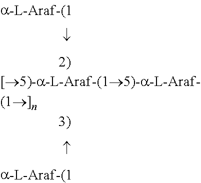 |
| B | 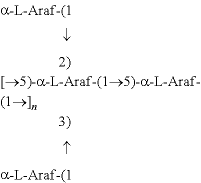 |
| C | 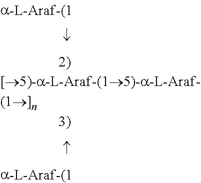 |
| D | 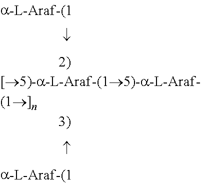 |
| E | 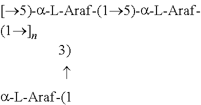 |
| F | 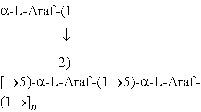 |

The linkages within arabinan required for immunomodulatory activity (based on the production of NO within a macrophage cell line) were determined (FIG. 11). Two commercially available linear arabinan polysaccharides with only a 1-5 backbone were devoid of immunomodulatory activity. A commercial arabinan polysaccharide with a 1-5 backbone and 1-3 branches also is devoid of activity. The inventors found that only the arabinan polysaccharide containing 1-5, 1-3 and also 1,2 linkages (18F) possessed immunomodulatory activity. The immunomodulatory activity of the 1-5, 1-3 and 1,2-linked arabinan polysaccharide (18F) was explored and was found to be concentration dependent, significantly increasing NO production in a macrophage cell line as the added concentrations increase (FIG. 12). Different preparations of the arabinan polysaccharide (all with the same 1-5, 1-3 and 1-2 glycosidic linkages and structure described in Formulas I and II above), but differing in the molecular weight prepared can differ in the extent of their activity. A preparation containing polysaccharides with 66 KDa and 10 KDa molecular weights had strong activity (Br/36/A; FIG. 13). A preparation containing polysaccharides with a 49 KDa molecular weight had moderate activity (Br/17/D). A preparation containing polysaccharides with a 12 KDa molecular weight also had moderate activity (18F). Finally, a preparation containing polysaccharides with a 3 Da molecular weight had poor/negligible activity (Br34F).

The linkage pattern of the arabinan (Br/14/E, less pure fraction) was determined according to De Castro et al (2010). Briefly, the sample (0.5 mg) was solved in DMSO (1 mL), treated with powdered NaOH, methylated with iodomethane (300 μL), hydrolyzed (2 M TFA, 200 μL, 120° C., 2 h), carbonyl-reduced with NaBD₄ (5 mg), and finally acetylated with acetic anhydride (50 μL) in pyridine (100 μL).

The PMAA derivatives were analyzed by GC-MS with an Agilent instrument (GC instrument Agilent 6850 coupled to MS Agilent 5973), equipped with a SPB-5 capillary column (Supelco, 30 m×0.25 i.d., flow rate, 0.8 mL min⁻¹) and He as carrier gas. Electron impact mass spectra were recorded with an ionization energy of 70 eV and an ionizing current of 0.2 mA. The temperature program used for all the analyses was the following: 150° C. for 5 min, 150→280° C. at 3° C./min, 300° C. for 5 min.

NMR Acquisition Parameters

NMR analyses were performed on a Bruker 600 MHz equipped with a cryogenic probe and spectra were recorded at 298 K. Acetone was used as internal standard (¹H 2.225 ppm, ¹³C 31.45 ppm) and 2D spectra (¹H-1H DQF-COSY, ¹H-1H NOESY, ¹H-1H TOCSY, ¹H-¹³C HSQC and ¹H-¹³C HMBC) were acquired by using Bruker software (TopSpin 2.0). Homonuclear experiments were recorded using 512 FIDs of 2048 complex with 32 scans per FID, mixing time of 100 and 200 ms were used for TOCSY and NOESY spectra acquisition, respectively. HSQC and HMBC spectra were acquired with 512 FIDs of 2048 complex point, accumulating 90 scans each, respectively. Spectra were processed and analyzed using a Bruker TopSpin 3 program.

Molecular Weight Determination of the Pure Arabinan Br/17/D

The molecular weight of the pure arabinan Br/17/D (equivalent of Br/18/F, see FIG. 2), was determined by SEC-HPLC (TSK gel G5000 PWXL, 30 cm×7.8 mm ID). In order to disclose the PM of the arabinan, 30 μl (1 mg/ml solution) were injected on the TSK column and eluted with 100% of 50 mM ammonium bicarbonate (Flow 0.8 ml/min). The eluate was monitored by refractive index. The column was calibrated with dextrans of known molecular weight (5 KDa, 50 KDa, 150 KDa, 410 KDa, 610 KDa), the data fitted in a linear regression and the molecular weight of Br/17/D evaluated accordingly (FIG. 2, Table 3).

TABLE 3
Molecular weight of the dextrans used to calculate the molecular
weight of the poly_1 and poly_2. Dextrans with molecular
weight from 5000 Da to 670000 Da were inject on the TSK column
(30 μl of a solution 1 mg /ml) and were used to construct
a calibration curve with the following straight-line equation:
y = −1.3958x + 15.176. Br/17/D was injected on
TSK column (30 μl of a solution 1 mg/ml) and the molecular
weight was determined by solving the straight-line equation.

| | MW(Da) | log MW | Time(min) | Elution volume |
|---|---|---|---|---|
| Dexstran | 5000 | 3.70 | 12.54 | 10.03 |
| Dextran | 50000 | 4.70 | 10.76 | 8.60 |
| Dexstran | 150000 | 5.18 | 9.90 | 7.92 |
| Dexstran | 410000 | 5.61 | 9.16 | 7.33 |
| Dexstran | 670000 | 5.8 | 8.85 | 7.08 |
| Br/17/D | 48977.88 | 4.69 | 10.79 | 8.63 |

### REFERENCE

De Castro C, Parrilli M, Holst O, Molinaro A. Microbe-Associated Molecular Patterns in Innate Immunity: Extraction and Chemical Analysis of Gram-Negative Bacterial Lipopolysaccharides. Methods Enzymol. 2010, 480, 89-115. Doi: 10.1016/S0076-6879(10)80005-9.

### Example 3—the Immune Modulatory Activity of Malvaceae

The inventors decided to study the immunomodulatory effects of plants from the Malvaceae family. They found that the extracts from Malvaceae induce a T_{H}1-mediated immune response.

Methods

Plant Materials and Extractions

Roots of Tilia cordata, Sparrmannia africana, Dombeya wallichii, Lagunaria patersonii, Pachira aquatic, Hibiscus syriacus, Hibiscus waimeae, Hibiscus rosa sinensis, Pavonia spinifex, Abutilon theophrasti and Sidalcea malviflora were kindly collected and donated by Belfast Botanic Gardens. Specimens of Thebroma cacoa, and Lavatera arborea were kindly donated by the Royal Botanic Garden, Edinburgh. Sida cordifolia roots were donated by Pukka herbs, Bristol. Malva sylvestris, Sphaeralcea coccinea, Gossypium hirsutum, Gossypium herbaceum, Malvastrum lateritium were grown from seeds, and Althea officinalis roots were obtained from Neal Yard Remedies, London.

Fresh plant roots were washed with isopropanol and water, roots were subsequently lyophilised and stored at −20° C. Lyophilised roots were homogenised into a fine powder using an analytical mill and 100 g of each root was macerated successively in n-hexane, chloroform, methanol and ddH2O, at room temperature for 24 h, in a successive manner. The n-hexane, chloroform and methanol extracts were concentrated in vacuo, using a rotary evaporator set at 45° C., the aqueous extract was centrifuged at 2000 g for 15 minutes and subsequently lyophilised. A precipitate was isolated from lyophilised aqueous extracts by dissolving 1 g of aqueous extract in 10-20 ml ddH20, followed by ethanol precipitation (abs.), the precipitate was pelleted by centrifugation which was later lyophilised and stored at −20° C., the isolated precipitate fractions were labelled at EXAP.

Lymphocyte Assays.

Preparation of Lymphocytes.

All animal procedures were carried out in accordance with animal Scientific Procedures Act (1986) and in accordance with the research ethic policies of Queen's University, Belfast. Adult female balb/mice were supplied by the Biological Response Unit at Queen's University Belfast were sacrificed by cervical dislocation. The spleen was aseptically removed and stored in 5 ml RPMI-1640 medium (Life Technologies™, Paisley), with 1% penicillin streptomycin (incomplete medium). The spleens were gently teased apart in 5 ml of incomplete media, using sterile forceps removing excess adipose tissue and the spleen capsule. The spleen was gently passed through a sterile 40 m nylon cell strainer (BD Falcon, Oxford) into incomplete medium using a rubber plunger, from a plastic 2 ml syringe. The cell strainer was washed with 15 ml of incomplete medium, bringing the total volume to 20 ml.

Large cell clumps and debris were removed after allowing media to settle for 10 minutes, and the supernatant was transferred to a fresh sterile 50 ml centrifuge tube. The cell suspension was then centrifuged at 300 g for 10 minutes and the supernatant was discarded. Erythrocytes were depleted with a red blood cell lysis buffer (1 ml volume spleen-1) (Sigma-Aldrich—Red Blood Cell Lysing Buffer Hybri-Max) for 1 min. The cells were then re-suspended in 50 ml RPMI 1640 Glutamax medium containing penicillin (100 U/ml), streptomycin (100 μg/ml) and 10% heat inactivated foetal bovine sera (FBS) (complete media). Viable cells were enumerated in 0.4% trypan blue dye using an automatic cell counter (Countess® Automatic Cell Counter, Invitrogen). Cell density was adjusted to 2×10⁶ cells/ml and 100 μl of the cell suspension was used to seed 96 well (BD Falcon) microtiter plate, resulting in 2×10⁵ cells/well. All of the above steps were carried out under sterile conditions in a biological safety cabinet to prevent any contamination. Cells were cultured at 37° C. in a 5% CO₂ humidified incubator for 72 h.

Lymphocyte Proliferation Assays.

Splenocyte refers to any of the different white blood cell types which constitute the splenic tissue. They consist of a variety of cell populations, part of both the innate and adaptive immune system including large populations of T and B lymphocytes. Logarithmic dilutions of extracts were made ranging from 2 mg/ml-20 ng/ml with complete RPMI medium in 1.5 ml Eppendorf tubes using a 2 mg/ml stock solution of extracts, 100 μl of each dilution was added to a seeded 96 well plate in triplicate, thus resulting in final concentration ranging 1 mg/ml-10 ng/ml. Concanavalin A (5 g/ml) isolated from Canavalia ensiformis (Sigma-Aldrich) a known mitogen of lymphocytes was used as a positive control (Beckert & Sharkey 1970).

Splenocyte proliferation was also measured using the AlamarBlue® cell viability indicator, which uses the reducing functionality of living cells to convert the resazurin to resorufin. Resazurin is a non-toxic, cell permeable compound that is blue, upon entering the cells. Resazurin is reduced to resorufin (pink/red) in viable cells (Page et al. 1993). Viable cells continuously convert resazurin to resorufin, thereby generating a quantitative measure of viability and cytotoxicity. In accordance with the manufacturer's instructions, 10% (20 l) AlamarBlue solution (Invitrogen™, Life Technologies, Paisley, UK) was added to the cell medium of each well, and incubated for a further 24 h.

The optical density of each well was measured at λ570 nm using a Tecan Safire2 microplate reader (Tecan, Switzerland). The results were expressed using the proliferation index (PI) using the formula PI=OD (λ570) stimulated cells/OD (λ570) non-stimulated cells.

Splenocyte proliferation was additionally confirmed using a Bromodeoxyuridine (BrDU) assay kit (Roche Diagnostics Ltd, Sussex). Briefly, BrDu is a synthetic analogue of thymidine is allowed to be incorporated into the DNA of replicating cells. Antibodies specific to BrDU can then be used to detect incorporated BrDU. Assays were performed in accordance with the manufacturer's protocol.

Humoral Immunity: Quantification of Immunoglobulin Secretion with ELISA.

The effects of extracts on the ability of splenocytes (B-Lymphocytes) to modulate immunoglobulin production was evaluated using a sandwich enzyme-linked immunosorbent assay (ELISA). Briefly, splenocytes were incubated with extract. After 48 h, 96-well plates were centrifuged at 300 g for 10 minutes and 150 μl of supernatants was carefully aspirated from each well, and transferred to another 96-well plate and stored at −20° C.

ELISA plates were coated with 75 μl of goat anti-mouse IgA, IgG and IgM antibodies (Sigma Alrich) diluted 1:1000 in 0.1 M sodium carbonate/bicarbonate buffer (pH 9.4) overnight at 4° C. The following day the plates were blocked with 150 μl 1% non-fat dried milk (marvel) for 2 h at 37° C.

Microtiter Plates (Greiner Bio-One) were coated with isotype-specific goat anti-mouse antibodies (IgA, IgM and IgA) (Sigma-Aldrich) at a concentration of 10 μg/ml carbonate buffer (0.1 M sodium carbonate/bicarbonate buffer, pH 9.4) and incubated overnight at 4° C. Thereafter, the plates were washed two times in wash buffer (50 mM Tris, pH 8.0, 0.1M NaCl, 0.05% Tween), wells were than coated with 150 μl blocking solution (1% non-fat dried milk, 50 mM Tris, pH 8.0, 0.15 M NaCl) and plate incubated for 2 h at 37° C. Following this, the plates were washed twice with wash buffer. Subsequently, 75 μl of the diluted cell supernatants were added to the wells of the 96-microtiter plate and incubated for 2 h at 37° C. The supernatants were discarded and plates were washed extensively with an ELISA wash solution. 75 μl of the anti-mouse IgG, IgA, IgM antibody, produced in goat (HRP-conjugated) (Sigma-Aldrich) was diluted at concentration of 1:2000, in a phosphate buffer with 1% non-fat dried milk and was added to each of the wells. The plates were incubated for 1 h on a plate shaker at 37° C. After incubation was completed, the plates were washed with the ELISA wash solution 3 times, the plate was further incubated with 100 μl of the horseradish peroxidase-conjugated goat anti-mouse per well for 60 min, after washing plate three times again the plate was developed by adding substrate solution containing 3,3, 5,5′ tetramethyl benzidine (TMB) (Sigma-Aldrich) to each well. The plates were then incubated at room temperature for 10 min. After incubation, the reaction was stopped by adding 100 μl of 2M sulphuric acid to each well and the absorbance λ450 nm, was measured using a Tecan Safire2 microplate reader.

Macrophages (RAW 264.7) Assays.

Macrophages are an imperative cellular component of the innate immune system, found in essentially all tissues where they patrol for materials deemed to be potentially pathogenic to the host. Upon detection of a potential pathogen they become activated and undergo drastic cellular changes. One study reported a change in 25% of observed macrophage genes (Ehrta et al. 2001). After activation, the cells morphologically appear larger, and exhibit an increase in endocytosis (pinocytosis and phagocytosis), and the hydrolytic proteins within lysosomes increase. There is a marked increase in the transcription, production and secretion of an array of products which would allow the macrophage to retain and eliminate a potential threat whilst orchestrating an adaptive immune response. The enzymes induced by activated macrophages include superoxide dismutase (this results in the formation of H₂O₂), NADPH oxidase (O2-) and inducible nitric oxide synthase (iNOS), an enzyme that metabolises arginine into NO for microbial killing (Mosser et al. 2008).

Preparation of Macrophages.

Murine macrophages from the RAW264.7 cell line were purchased from European Collection of Cell Cultures (ECACC). The cells were grown in 75 cm culture flasks, in Dulbecco's modified Eagle's medium (DMEM), containing 10% heat-inactivated FBS, 100 u/ml penicillin and 100 μg/ml of streptomycin. The cells were incubated in the presence of 5% CO₂ at 37° C. All cell culture work was carried out in sterile conditions in a biological safety cabinet to prevent any contamination.

Macrophage Proliferation Assay.

Cells were seeded in 96 well plates at a seeding density of 1×10⁵ cells ml (1×10⁴ cells/well). Lipopolysaccharide (LPS) (Sigma-Aldrich) a known activator of macrophages at a concentration of 1 μg/ml was used a positive control. Incubation time for assays was 24 h. Cells were cultured in the presence of 5% CO2 at 37° C. and macrophage proliferation was measured using AlamarBlue.

Measurement of Nitrite Concentration.

Induction of iNOS results in the endogenous production of nitric oxide (NO) by conversion of L-arginine to citrulline (Murad 1998). NO is a key facilitator of inflammatory responses, and is only produced by macrophages activated through the classical pathway (ligation of TLRs) (Mosser 2003). Due to the highly reactive nature of NO, its formation is commonly indirectly measured (i.e. by measuring nitrite (NO2-), one of two primary stable non-volatile breakdown products of NO).

Nitrite (NO₂—) concentration in culture medium was measured using the Griess reaction method, in accordance with the protocol provided by the manufacturer (Life Technologies, Paisley). Briefly, to determine the amount of NO₂— produced, 50 μl of medium from each well was aspirated and transferred to a sterile 96-well plate together with 50 μl sulfanilic acid 1% (10 mg/ml) in 5% phosphoric acid. The plate was then incubated at room temperature for 10 minutes. Thereafter, 50 μl of a solution consisting of 0.1% N-(1-naphthyl) ethylenediamine dihydrochloride was added and incubated for an additional 20 minutes at room temperature. The concentration of NO2- was measured colourimetrically at λ550 nm using a Tecan Safire2 microplate reader.

A standard solution of 0.1M sodium nitrite (NaNO₂) was used to make serial dilutions in the range of 100-1 μM to quantitate NO2- concentrations by means of a standard curve.

Neutral Red Phagocytosis Assay.

The ability to measure endocytic activity of macrophages through the neutral red uptake assay was described by Antal et al. (1995). A 0.1% neutral red (NR) (3-amino-7 dimethylamino-2-methylphenazine hydrochloride) solution was prepared by dissolving 0.1 g neutral red crystals in 100 ml sterile phosphate buffer (pH7.2), the solution was than filtered using a syringe filter, thus removing and insoluble crystals, thus ensuring homogeneity of solution. Briefly RAW 264.7 cells were cultured and seeded in a 96-well plate and incubated for 24 h, thereafter 20 μl of the sterile NR solution was added to each well (Repetto et al. 2008).

Plates were than incubated for an additional 6 hours. Following this, supernatant was carefully aspirated and discarded, wells were subsequently washed twice with 200 μl PBS (PBS: 1M KH₂PO₄, 1M K₂HPO₄, 5M NaCl pH7.2). Cells were lysed by adding 100 μl of cells lysis solution (50% abs.ethanol, 49% deionised water and 1% glacial acetic acid) cells were incubated in room temperature for 2 hours and the optical density at 540 nm was measured using the Tecan Safire2 microplate reader.

Endotoxin Detection.

Compared to proteins, endotoxin (a lipopolysaccharide) is very stable, resistant to extreme temperatures and pHs. Endotoxin is derived from the cell membrane of Gram negative bacteria, and linked within the matrix of bacterial cell walls. However, as bacteria grow and divide, they continuously release endotoxin units into the environment. These endotoxin units are capable of activating a TH1-mediated immune response, independently of the IFN-γ priming signal. Thus, LPS can directly activate macrophages (Andrade et al. 2003) resulting in the release of inflammatory mediators, such as TNFα, IL-6 and IL-1, thus the presence of lipopolysaccharides in extracts has to be eliminated.

Exposure of Limulus spp blood to endotoxin causes clotting (Bang & Frost 1953), a mechanism which acts to entrap and immobilise bacteria, preventing systemic dissemination of bacteria. This discovery became the foundation of the Limulus Amebocyte Lysate (LAL) assay (Young et al. 1972), in which exposure of endotoxin to lysates of amebocytes results in the initiation of a cascade of enzymes culminating in the activation of a clotting enzyme, that cleaves coagulogen (clotting protein), resulting in the formation of an insoluble (coagulin), which consequently coalesces forming a clot (Levin & Bang 1964).

LAL Assay.

Pyrosate® (CapeCod) rapid endotoxin detection kit (0.03 endotoxin units (EU)/ml) is a highly sensitive detection method. The kit is endotoxin specific for glucan concentrations of up to 100 ng/ml. The assay was performed in accordance with the manufacturer's protocol. Briefly, 1 mg/ml of lyophilised aqueous extract was dissolved in sterile water, endotoxin free (Sigma), the stock solution was further diluted to produce a 100 ng/ml solution, 0.5 ml of the 100 ng/ml solution was than dispensed into a tube containing 0.5 ml lyophilised LAL reagent, which only consists of an aqueous extract of L. polyphemus amebocytes, salts, glucan blocker and a buffer. The test tube was then gently mixed in a horizontal plane for 20-30 seconds, 2.5 ml was then transferred to a tube containing the control standard endotoxin (E. coli 0113:H10). The tubes where than gently mixed again for 20-30 seconds in a horizontal plane, followed by incubation at 37° C. for 1 h.

An In Vitro Immune Model (Galleria mellonella Larvae).

Many essential aspects of the innate immune response to microbes are highly conserved between arthropods and gnathostomes. This striking homology in immune responses has resulted in much interest in using insects as an archetypal model to study host-microbe interactions (Hultmark 1993). Arthropods are a highly economical alternative to gnathostomes and a more ethically acceptable model. Larvae of the greater wax moth Galleria mellonella (Lepidoptera) has an additional advantage, as growth optimum of the larvae is 37° C., also the optimum in which to incubate a diverse range pathogenic microorganism known to infect gnathostomes (Ramarao et al. 2012).

The innate immune response is facilitated through the haemolymph system in arthropods, on detection of PAMPs. In Galleria spp, as with all arthropods, the immune response involves a humoral and a cellular response (Vilmos & Kurucz 1998). The humoral response includes the production of small antimicrobial peptides and a coagulation cascade mediated through enzymes, homologous to the one described previously for Limulus spp. (Muta & Iwanaga 1996). Haemolymph in Lepidoptera larvae contain primarily granular cells and plasmatocytes (Lavine & Strand 2002), and on detection of PAMPs they respond by encapsulating the potential threat or eradicating it through phagocytosis (Schmit & Ratcliffe 1977), interestingly these cells also display receptors analogous to those that are found on phagocytic cells in gnathostomes (Vilmos & Kurucz 1998).

Galleria mellonella

Galleria mellonella larvae (Livefoods Direct) were reared on an artificial diet 25° C. (dark), during experiments larvae were kept in an incubator at 37° C., in sterile Petri dishes (5 cm). Experimental groups consisted of 10 larvae (last instar) weighing 250-300 mg.

Bacterial Load in Larvae Pre-Exposed to Aqueous Extract of Malveae.

Solutions of the aqueous extracts of Sida cordifolia, Malva sylvestris, Malvastrum lateritium, and Sidaalcea malviflora at concentrations of 1 mg/ml and 0.1 mg/ml were prepared in PBS in sterile conditions. Terumo Myjector 1 ml 29G 0.33×12 mm were used to inject 10 μl aliquots of extracts into the hemocoel through proleg (left) in dorsolateral region of larvae in each group, the negative control group was injected with PBS (150 mM NaCl; 5 mM KCl in 0.1 M Tris-HCl, pH 6.9), larvae were placed in an incubator set at 37° C. for 24 h. Each group was then infected with MRSA (ATCC43300) or E. coli (ACTCC 11303). Inoculation experiments were carried out in accordance with the protocol described by Ramarao et al. (2012). Inoculum density was standardised to the optical equivalent of the 0.5 McFarland turbidity standard, which at λ600 nm absorbance of 0.06-0.10 nm, corresponding to 107 cfu/ml (Andrews 2001), was further diluted in PBS (1:10), inoculum was administered through a proleg (right) in dorsolateral region, in a similar manner to that of aqueous extracts. Inoculated larvae were maintained in an incubator set at 37° C. for 24 h.

Bacterial inoculums were serially diluted to verify inoculum size using the Miles and Misra Method (Miles et al. 1938), which involves 20 μl of each dilution being plated out on Muller-Hinton agar plates and incubated at 37° C. for 24 h. Inoculum concentration was therefore calculated as 1.2×10⁶ CFU/ml. At 24 h, 48 h and 72 h post inoculation, 3 larvae from each group were removed and the bacterial load in haemolymph determined using the Miles and Misra Method to calculate CFU.

Immune Response.

The effects of malveae extracts on haemocytes was evaluated by injecting aqueous extracts of Sida cordifolia, Malva sylvestris, Malvastrum lateritium, and Sidaalcea malviflora at concentrations of 1 mg/ml and 0.1 mg/ml. Larvea were subsequently maintained in an incubator set at 37° C. for 24 h. After incubation, the haemolymph was removed and haemocyte were enumerated using a haemocytometer.

Polysaccharide Analysis.

The presence of polysaccharides in the alcohol precipitate was confirmed by the Molisch's reagent (5% thymol dissolved in alcohol (abs.). Briefly, 10 mg samples where dissolved in 750 μl of ddH₂0 in a glass test tube to which 500 μl of the Molisch's reagent was added, followed by the addition of 3 gtt of concentrated sulphuric acid.

Determining Protein Content in Polysaccharide Enriched Fractions.

As the method for precipitation of polysaccharide can also precipitate proteins. Therefore, the protein content in precipitate had to be determined. A number of methods have been developed for determining protein content in physiological fluids obtained from animal sources, unfortunately these assays have been developed based on protein-copper chelation, which results in the reduction of copper from Cu (II) to a Cu (I), and can be influenced by the presence of reducing agents that are present in plant extracts (Compton & Jones 1985). In contrast, the Bradford assay is based on the formation of a complex between Brilliant Blue G250 (Coomassie Brilliant Blue) dye and between basic amino acid residues (arginine, lysine and histidine). The resulting complex results in a shift in the absorption maximum of Brillant Blue G250 from λ465 to 595 nm (Bradford 1976).

Briefly, polysaccharide enriched fractions were dissolved in PBS (0.5 mg/ml), 10 μl of the 0.5 mg/ml fractions were aliquoted in triplicate to wells of a 96-well plate in which 250 μl of the Bradford reagent (Bio-Rad) was added at room temperature. A protein standard of 1 mg/ml bovine serum albumin (BSA) (Sigma Aldrich) in PBS, which was subsequently serially diluted (1:10) to form standards ranging from 0-100 μg/ml. 10 μl was added to the wells of a 96-well plate in triplicate along with samples followed by 150 μl of the Bradford regent. The plate was incubated at room temperature for 20 min and absorbance measured at λ595 nm, using a Tecan Safire 2 microplate reader (Tecan, Switzerland).

Infra-Red Spectroscopy.

Raman-IR spectroscopy (Thermo Scientific Nicolet iS5 FT-IR Spectrometer with Omnic Software™, Madison, Wisconsin, USA) was performed followed by ¹³C NMR and ¹H NMR in MeOH using a 400 MHz Bruker NMR (Billerica, MA, USA) to verify the structure.

GC-MS Analysis of Polysaccharides.

The polysaccharide enriched fractions (100 mg) were hydrolysed with 10 ml 1M trifluroacetic acid (TFA) (Sigma-Aldrich) at 105° C. for 7 h in a closed 25 ml flask, (Uzaki & Ishiwatari 1983) and then subsequently lyophilised. Thereafter samples (2 mg) were derivitised by a silylation reaction with 500 μl N,O-Bis(trimethylsilyl)trifluoroacetamide (BSTFA) with 1% trimethylchrosilane (Sigam-Aldrich) in 1 ml anhydrous pyridine. The reaction was carried out at room temperature for 12 h.

GC-MS analysis was performed using gas chromatography (Agilent 7890A) interfaced with a mass selective detector (Agilents 5975C), with a ZB semi-volatiles column (30 m×0.25 mm×0.25 m Zebron™, Phenomenex Inc) with helium as the carrier gas at a constant rate of 1 ml/min. The injector and MS source temperatures were maintained at 260° C. and 230° C., respectively. The column temperature program consisted of injection at 80° C. and hold for 1 min, temperature increase of 15° C. min-1 to 300° C., followed by an isothermal hold at 300° C. for 15 min. The MS was operated in the electron impact mode with an ionisation energy of 70 eV. The scan range was set from mass scan range was 50-550 Da. Injection volume was 1 μl, inlet had a split flow of 20 ml-1 (split ratio 20:1).

Data was acquired and processed with the Chemstation software (Hewlett Packard). Compound identification was performed by comparison with chromatographic retention characteristics and mass spectra of standards, and the NIST mass spectral library (National Institute of Standards and Technology, USA) (Magalhaes et al. 2007).

Results.

Splenocyte Proliferation

Results from the proliferation assays performed with splenocytes are summarised in a heat map (FIG. 14). Results show that crude aqueous extracts of the malveae tribe of the malvoideae significantly stimulated splenocytes proliferation (P<0.05) in a concentration-dependent manner, from 0.01 to 1 mg/ml. This activity was observed among all 8 species representing the different genera of the malveae tribe (subfamily malvoideae). Splenocyte proliferation was also observed in the aqueous extracts of the Gossypieae tribe and among one of the species (Abelmoschus moschatus) of the Hibisceae tribe. No activity was observed in any of the n-hexane, chloroform or methanol extracts suggesting that source of this activity arose from a highly polar constituent(s). The observed splenocyte stimulatory activity observed in this assay was subsequently confirmed by means of the BrdU assay (FIG. 14) which resulted in a similar concentration-dependent effects being observed in the aqueous extracts of the malveae.

Antibody Secretion

Supernatants collected from wells with splenocytes incubated with extracts of the malvaceae, showed a 2- to 3-fold increase in total IgG levels (FIG. 15), post-incubation with all the aqueous extracts of species from the malveae tribe, at a concentration of 1 mg/ml and a 1- to 2-fold increase at concentrations of 0.1 mg/ml. A rise of 1.2-1.5-fold in total IgG levels was also detectable in supernatants incubated with Sida cordifolia, Sidalcea malviflora and Althaea officinalis at a concentration of 0.01 mg/ml (FIG. 21). A similar trend was observed for total IgM levels post-incubation, which similarly resulted in a 2- to 3-fold increase in IgM levels at a concentration of 1 mg/ml and 1.3- to 2.5-fold increase in IgM levels at a concentration of 0.1 mg ml₋₁ (FIG. 21). Total levels of IgM which is summarised in a heat map in FIG. 17. There was a 1.2 to 1.7-fold rise at a concentration at a concentration of 1 mg/ml, in total IgA in supernatants aspirated from wells in which cells were exposed to aqueous extracts of the malveae. Total levels of IgA are summarised in a heat map in FIG. 16.

Macrophage Activation

A proliferation assay was carried on RAW 264.7 macrophages to observe proliferation. No proliferation was observed however, morphological changes typical of macrophage activation were observed (FIG. 18). Cells incubated with aqueous extracts from malveae appeared to be much larger and less uniform compared with cells where no extract was added (negative control). This is consistent with activated macrophages because of their increased membrane projections and increased number of vacuoles for phagocytosing internalised pathogens, which collectively increases cell size.

Respiratory Burst

Activated macrophages release reactive oxygen species and reactive nitrogen species. Nitric acid which rapidly forms nitrite ions (NO₂—) is one such nitrogen species which indicates increased production of reactive species essential for the degradation of internalised parasites in macrophages. The heat map in FIG. 19 summarises the responses in NO₂⁻ production when splenocytes are incubated with extracts. Nitrite levels followed a similar trend to those of splenocyte proliferation and IgG, IgM and IgA levels as previously reported. In summary, levels of NO2⁻ on incubation of macrophages with aqueous extracts of malveae species at a concentration of 1 mg/ml ranged between 60-80 μmol/ml, which was the same as LPS at a concentration of 1 ng/ml. This activity was statistically significant (P<0.05) even at a concentration of 0.001 mg/ml where detectable nitrates levels were found to range 10-20 μmol/ml.

Phagocytosis

An additional characteristic of activated macrophages is their ability to phagocytose. Macrophages were exposed to aqueous extracts of Sida cordifolia (malveae). Post incubation a neutral red solution was added and incubated for 6 h. Cells which appear to have elevated NO₂⁻ levels also appeared to have a cytoplasm which had a more intense red colour, reflecting their phagocytic abilities. On aspiration of supernatant and subsequent lysis of cells with a lysis solution a 2-fold increase in phagocytic activity was observed on incubation of cells with aqueous extracts of the malveae, which is summarised in heat map FIG. 20.

Analysis of Mucialgenous Matter in Aqueous Extracts of the Malveae

The aqueous extracts were lyophilised and mucialgenous matter extracted with ethanol. The precipitate was collected, lyophilised and quantified. The content of mucilage in aqueous extracts of tested malveae was found to be in the range of 12-24% (FIG. 23).

Analysis of Polysaccharide Enriched Fractions (EXAP)

The presence of mucilaginous polysaccharides in the ethanol precipitate was initially confirmed initially by the Molisch's test. The presence of polysaccharides was confirmed by observing the immediate formation of pink/purple menisci on the interface of thymol solution and aqueous solution following the addition of conc. H₂SO₄. No colour change was observed with the Lugol's solution suggesting the absence of starch. Also, a negative result was observed for reducing sugars with the Benedict's reagent. The presence of polysaccharides in lyophilised precipitates was subsequently confirmed using infrared spectroscopy. FIG. 24 shows the spectra of atypical polysaccharide precipitates, which represents IR spectra produced by ethanol precipitates of aqueous extracts of the malvaceae. In summary, the maximum observed in the 3380 cm-1 region is representative of OH groups, whereas the maximum in the range 2930 cm-1 indicates the presence of —CH of —CH2, H—C═O present and the maximum observed in the 1650-1700 cm-1 regions indicate the presence of C═O groups overlapping with maximums representative of C═C. Maximums at 930 and 615 cm-1 typically related to sugar cycles were also observed, in all IR spectra. Typically, all spectra had a region between 1200-1000 cm-1 which was dominated by sugar ring vibrations overlapping with stretching vibrations due to presence of C—OH side groups and the C—O—C glycosidic bonds vibrations.

Protein Content in Polysaccharide Enriched Fraction (EXAP)

Since ethanol precipitation of polysaccharides can also result in protein precipitation the protein concentration was determined using the Bradford assay. Protein content was determined to be in the region of 0-0.1 μg/ml in a solution constituted of 1 mg ml-1 of precipitated polysaccharides (i.e. <0.0001% w/v).

GC-MS Analysis of Polysaccharide Precipitate (EXAP)

Hydrolysed polysaccharides were lyophilised and after derivatisation were subjected to GC-MS analysis and monosaccharides that constituted polysaccharides were identified using standards and the NIST library. There were distinct differences in the monosaccharide profiles between the malveae tribe and members of the subfamilies which did not exhibit immunomodulatory activities. In summary, polysaccharides from the malveae tribe contained a greater diversity of monosaccharides, in all cases containing a total of 9 different monosaccharides. Generally, polysaccharides of the malveae were found to be made of rhamnose (8-18%) glucose (16-28%) mannose (5-12%) arabinose (2-9%) galactose (28-41%) and uronic acid (1-14%) (FIGS. 25 and 26) in contrast polysaccharides isolated from the aqueous extracts of species from other subfamilies appeared to have a different composition (FIG. 27).

Detection of Endotoxins with the LAL Test

The contamination of Gram-negative bacterial lipopolysaccharides (endotoxins) was determined with the LAL test. Levels of endotoxins in aqueous extracts of the malveae and the alcohol precipitates, were negligible at level of 0.03 EU/ml.

Immunomodulatory Activities of the Polysaccharide Enriched Fractions (EXAP)

The lyophilised precipitates of the malveae, which were shown to trace amounts of proteins and negative for the presence of endotoxins were tested alongside the crude aqueous extracts from which they were derived.

Effects of the EXAP on Splenocyte Proliferation, Immunoglobulin Secretion and Macrophage Activation

Aqueous extract (EXA) from the malveae gave similar responses for splenocyte proliferation, immunoglobulin secretion and macrophage activation as previously mentioned. However, significant increases in activity were observed for the polysaccharide precipitates (EXAP) compared with EXA. Greater splenocyte proliferation was observed with EXAP compared with EXA (FIG. 28). Greater IgG, IgM and IgA secretion was also observed with the EXAP compared to the EXA (FIGS. 29, 30 and 31). Macrophage activation was also greater both in terms of phagocytic activity and NO2- production with EXAP compared with EXA. This indicates that there was enrichment of this bioactivity when polysaccharides were enriched (FIGS. 32 and 33).

### DISCUSSION

In this study, the inventors identified that extracts from plants of the malvaceae family induce immunomodulatory activity in vitro and in vivo. They also identified major constituents of the extracts. A range of plants which are representative of the broader malvaceae family were screened for their ability to activate elements of the innate and adaptive immune system. Aqueous extracts belonging to the malveae tribe of the malvaceae family enhanced murine lymphocyte function as evidenced by an induction of proliferation and antibody secretion. In addition, aqueous extracts of the malveae species enhanced phagocytic activity and respiratory burst activity in macrophages. The immunomodulatory activity appears to be triggered by the polysaccharide constituents of the malveae and these cause immunomodulation in a simple in vivo model. Most promisingly, pre-treatment of a larvae host with malveae extract enabled it to substantially resist infection to a potentially fatal microbial challenge. One of the most promising alternatives to prophylactic antibiotics to have emerged since the emergence of widespread antibiotics resistance is the use of immunemodulators which include exogenously derived biological response modifiers (EDBRMs) from plants. EDBRMs are chemically diverse and include steroidal lactones, phenylchromones, triterpenoids, bis-benzylisoquinoline among others, but the most prominent group chemically to have emerged as EDBRMs are the polysaccharides. The proposed mechanisms by which plant-derived polysaccharides are postulated to instigate immunomodulatory responses is by triggering one or more of a diverse range of pattern recognition receptors expressed by the immune system.

Immunomodulatory activity was found to be only present in aqueous extracts, and completely absent from hexane, chloroform and methanol extracts. This indicates that the active constituent is polar in nature. The inventors considered the possibility that activity could be the result of endotoxin contaminants from Gram-negative bacteria, which can commonly contaminate biological samples. The presence of endotoxin contaminants was immediately ruled-out by means of the LAL assay. Ethanol precipitation resulted in the isolation of pale-brown precipitates from the aqueous extracts which were confirmed as the source of this immunomodulatory activity. Molisch's test indicated the presence of polysaccharides and this was confirmed by IR spectroscopy. The protein content of EXAP fractions was found to be negligible and therefore unlikely to be responsible for immunomodulatory activity. Total polysaccharide content (% wt.) of aqueous extracts correlated with observed immunomodulatory activity of the Malvaceae family as a whole. The profiles of polysaccharides across the Malveae tribe demonstrated that they contain the same 9 monosaccharides, and although there were some differences in their ratio this does not substantially affect their immunomodulatory activity. However, those species from tribes within the same malvoideae subfamily which were devoid of immunomodulatory activity had quite different monosaccharide profiles. For example, in Hibiscus rosasinensis there were only 6 monosaccharides present of which 96% of monosaccharides content comprised of just three sugars, namely glucose (67%), galactose (21%) and arabinose (8%). This is much less diverse than Sidalcea malviflora for example which contains glucose (22%), arabinose (8%), rhamnose (18%), ribose (2%), xylose (1%), galacturonic acid (1%), galactose (28%), mannose (6%), uronic acid (14%). Similarly, Lagunaria patersonnii from Bombacoideae subfamily which was also devoid of activity had a much less diverse monosaccharide profile. Only 6 monosaccharides were present and 94% of all monosaccharides content again comprimises of just three sugars, namely glucose (33%), galactose (50%), and galacturonic acid (9%). Monosaccharide composition of polysaccharides is crucial for the immunemodulating activity. The vast majority of plant derived polysaccharides appear to modulate the immune system through the activation of macrophages (Schepetkin & Quinn 2006). The results of this Example demonstrate the ability of polysaccharides isolated from Malveae to activate several macrophage functions in vitro. The concentration-dependent release of NO by activated macrophages suggests that macrophages are activated classically, as macrophages activated alternatively would not result in the induction of iNOS. Furthermore, the classical activation of macrophages is highly suggestive of effector cells which are directing the immune system towards a T_{H}1 mediated response. This typically results in the activation of microbicidal activities against bacteria, fungi, protozoa, and viruses. These responses result in activation of macrophages, which leads to production of reactive oxygen species and reactive nitrogen species via the induction of iNOS and increase in the pinocytic rate observed in this Example. Once activated, macrophages begin to secrete cytokines which induce a T_{H}1 mediated immune response, in which naïve CD4+ T cells will differentiate into T_{H}1 effector cells, resulting in the secretion pro-inflammatory cytokines. These pro-inflammatory cytokines subsequently initiate the activation of the more specific adaptive immune response in gnathostomes for instance activation of macrophages results in the expression B7-1 (CD80) and B7-2 (CD86) proteins which act as a costimulatory signal essential for the activation of T lymphocytes (CD8+), which is an essential component of the adaptive immune response. Galleria mellonella larvae were used as a primitive arthropod model for studying the stimulation of innate immunity in arthropods, as they also have the capacity to detect highly conserved PAMPs. Using the larvae model, the stimulatory effects of the polysaccharide-enriched fractions of the Malveae family on the innate immune response were demonstrated. This was evident by the increase in haemocytes (FIG. 34), and by the ability of the galleria larvae to resist infection and rescue bacterial load (FIG. 35), considering the polysaccharide enriched fractions was shown to have no direct antimicrobial activity (≤16 mg/ml). Therefore, the only plausible explanation for the decrease in bacterial load observed is the direct activation of innate immune system. Characteristic of T_{H}1 effector cells are the cytokines TNF-α and IFN-γ. TNF-α is implicated as an essential factor in wound healing. It induces fibroblast proliferation and angiogenesis. This may explain the traditional application of malveae genera as facilitators of wound healing. IFN-γ, on the other hand, induces B cell production of antibodies for opsinization and complement-fixation. The increase in immunoglobulins (IgG, IgM and IgA) in this Example could be a consequence of increased levels of IFN-γ. The increase in total antibody levels and stimulation of innate immune response may be responsible for the traditional use of genera of the Malvea genera as anti-venomous activities associated with the Malvea tribe.

Finally, it is worth noting that the polysaccharides of the Malveae had a very potent effect on the proliferation of murine splenocytes which is comprised B cells (50-60%), T cells (30%) and macrophages (5%), by the polysaccharides of the Malveae isolated from aqueous extracts of the Malveae. Proliferation assays with the macrophage cell line (RAW 264.7) did not show proliferation so changes in population sizes would have largely have arisen among B and T cells.

### Conclusion

Polysaccharides derived from plants are relatively non-toxic and tend not to induce the anaphylaxis associated with polysaccharides derived from microbial sources. In this Example it has been demonstrated that polysaccharides isolated from species of Malveae can have significant immunomodulatory activities, which possibly result in the stimulation of antigen presenting cells and the concomitant promotion of the T_{H}1 pathway. The activation of the T_{H}1 pathway promotes increased biocidal activities which would help to rapidly eliminate an infection. This ability was verified by in vivo bacterial challenge of an arthropod model of the innate immune system. It is clear that plants from the Malveae subfamily can be used to prevent or resist bacterial infection and as such could offer considerable therapeutic potential.

### Example 4—Immunomodulatory Activity of Sida cordifolia

The inventors fractionated and characterised the polysaccharide enriched fraction from the aqueous extract of Sida cordifolia. They performed bioactivity-guided fractionation of the polysaccharides, identified some of the monosaccharides of the polysaccharide, and explored the effects of the purified extract on the toll-like-4 receptor (TLR4). They found that the purified extract does not activate the TLR4 pathway.

Methods

Extractions of Plant Materials

Sida cordifolia L radix was collected in Karnataka, India (2012), and donated by Pukka Herbs, Bristol, a voucher specimen was deposited in the DBN Economic Collections, Glasnevin Herbarium Dublin (DBN 06:201261). Plant roots were washed with isopropanol and water. The lyophilised roots were homogenised to a fine powder using an IKA® A11 analytical mill (IKA® Werke GmbH & Co. KG, Staufen, Germany). Powdered (200 g) was macerated in n-hexane, chloroform, methanol and ddH2O, at room temperature for 24 h, in a successive manner. The n-hexane, chloroform and methanol extracts were concentrated in vacuo, using a rotary evaporator set at 45° C., the aqueous extract was centrifuged at 4500 rpm for 15 minutes and subsequently lyophilised, extracts weighed 0.092 g, 54.32 g 16.4 g, and 62.2 g respectively. The polysaccharides in the lyophilised aqueous extract (62.2 g) were extracted from the lyophilised aqueous extract by dissolving 10 g in 20 ml ddH20, followed by ethanol precipitation (abs.) (1:4 v/v) overnight. The resulting precipitate was pelleted by centrifugation, and supernatant discarded. The yield of the lyophilised pellet was 2.356 g (23.56% of lyophilised aqueous extract).

Fractionation of Polysaccharides.

The crude polysaccharide fraction obtained above (2.00 g) was further fractionated using DEAE™ Sephadex A-50 (weak anion exchanger) (GE Healthcare) column (30×2.5) pre-equilibrated with ddH20. Elution was stepwise using NaCl solutions of increasing ionic strength low (0 mol/l), medium (0.75 mol/l) and high (2 mol/l) NaOH. Resulting fractions were lyophilised and weighed, yielding 0.089 g, 0.251 g and 1.412 g, respectively. Fractions were further fractionated according to molecular size, using Vivaspin™ molecular weight cut-off (MWCO) filters (Sartorius, Goettingen Germany). Briefly, lyophilised extracts were dissolved at concentrations of 50 mg/ml in ddH20 and loaded onto a 100 kDa MWCO filter and centrifuged (2000 g; 1 h). Residue remaining in the 100 kDa MWCO filter was collected in a 1.5 ml Eppendorf tube by dissolving (200 μl) and rinsing (100 μl) with water. This procedure was repeated for all ion exchange chromatographic fractions (low, medium and high). Eluents from 10 kDa MWCO filters were collected but subsequently discarded once it was determined that all were inactive.

Fractions resulting from the above process were: SCAF0 (crude polysaccharide fraction of S. cordifolia); SCAF 1: low ionic strength and <100 kDa (56.1 mg); SCAF2: medium ionic strength and between 10-100 kDa (142.6 mg) SCAF 3 medium ionic strength and <100 kDa (72.5 mg); SCAF 4 high ionic strength (2 mol/l) and between 10 kDa-100 kDa (532.6 mg) and SCAF 5: high ionic strength and <100 kDa (786.7 mg).

Lymphocyte Cell Preparation.

All animal procedures were conducted in accordance with animal Scientific Procedures Act (1986) and in accordance with the research governance policies of Queen's University Belfast. Adult female balb/mice were supplied by the Biological Response Unit at Queen's University Belfast and were sacrificed by cervical dislocation. The spleen was aseptically removed and stored in 5 ml RPMI-1640 medium with 1% penicillin streptomycin, but with no foetal calf serum. Spleens were gently teased apart in 5 ml of media, using sterile forceps removing excess adipose tissue and the spleen capsule. The spleen was pressed through a sterile 40 m nylon cell strainer into the RPMI 1640 medium and lymphocyte cell suspensions were prepared. Viable cells were enumerated in 0.4% trypan blue using an automatic cell counter (Countess® Automatic Cell Counter, Invitrogen). Cell density was adjusted to 2×10⁶ cells/ml. 100 μl of the cell suspension was used to seed 96 well (BD Falcon) microtiter plate, resulting in 2×10⁵/well. All of the above was carried out under sterile conditions in a biological safety cabinet to prevent any contamination at 37° C. in a 5% CO₂ humidified incubator for 72 h.

Lymphocyte Proliferation Assays.

Logarithmic dilutions of S. cordifolia fractions SCAF0-SCAF5 were made in a concentration range of 0 ng/ml-2 mg/ml in RPMI medium with 10% fetal calf serum, using a 2 mg/ml stock solution of extracts. Each dilution (100 l) was added to a seeded 96-well plate in triplicate, thus resulting in final concentrations ranging 1 mg/ml-10 ng/ml. Concanavalin A (5 μg/ml) isolated from Canavalia ensiformis (Sigma-Aldrich) a known mitogen of lymphocytes was used as a positive control (Beckert & Sharkey 1970). Splenocyte proliferation was measured using AlamarBlue. According to manufacture instructions, 10% (20 l) AlamarBlue solution (Invitrogen™, Life Technologies, Paisley, UK) was added to the cell medium to each of the well, and incubated for another 24 hours. The optical density of wells was measured at λ570 nm using a Tecan Safire2 microplate reader (Tecan, Switzerland). The result were expressed using the proliferation index (PI) using the formula PI=OD (λ570) stimulated cells/OD (λ570) non-stimulated cells.

Humoral Immunity, Quantification of Immunoglobulin Secretion with ELISA.

The effects of polysaccharide fractions SCAF0-SCAF5 on splenocytes (B-Lymphocytes) ability modulate to immunoglobulin production was evaluated using a sandwich enzyme-linked immunosorbent assay (ELISA). Briefly, splenocytes were incubated with extract. After 48 hours, 96-well plates were centrifuged at 300 g for 10 minutes, and then 150 μl of supernatant was carefully aspirated from each well, and transferred to another 96-well plate and stored at −20° C. An ELISA assay was performed for IgA, IgG and IgM antibodies. After incubations were complete plates were washed 3 times with the ELISA wash solution, the plate was further incubated with 100 μl of the horseradish peroxidase-conjugated goat anti-mouse per well for 60 min. After further washing, the plate was developed by adding substrate solution containing 3,3, 5,5′ tetramethyl benzidine (TMB) (Sigma-Aldrich) per well. The reaction were stopped by adding 100 μl of 2M sulphuric acid per well and the absorbance (λ450 nm), was measured using a Tecan Safire2 microplate reader.

Cell Culture (RAW 264.7).

Murine macrophages from cell line RAW264.7 were purchased from European Collection of Cell Cultures (ECACC). The cells were grown in 75 cm² culture flasks, in Dulbecco's modified Eagle's medium (DMEM), containing 10% heat inactivated FBS, 100 u/ml penicillin and 100 μg/ml of streptomycin. The cells were incubated in the presence of 5% CO₂ at 37° C. All cell culture work was carried out in sterile conditions in a biological safety cabinet to prevent any contamination.

Macrophage Proliferation Assay.

Cells were seeded in 96-well microtitre plates at a seeding density of 1×10⁵ cells ml (1×10⁴ cells/well) with fractions of S. cordifolia fraction SCAF0-SCAF5 at concentrations ranging from 1 mg/ml-10 ng/ml. Lipopolysaccharide (LPS) (Sigma-Aldrich) a known activator of macrophages (1 μg/ml) was used a positive control. Plates were incubated (5% CO₂; 37° C.; 24 h) and macrophage proliferation was measured using AlamarBlue (Invitrogen™, Life Technologies, Paisley, UK).

Measurement of Nitrite Concentration.

To determine the amount of produced NO₂— produced, 50 μl of medium from each well of the 96-well microtitre plate was aspirated post incubation and transferred to a fresh 96-well microtiter plate to which a solution of 50 μl sulfanilic acid 1% (10 mg/ml) in 5% phosphoric acid was added and incubated for 10 minutes at room temperature. Thereafter, 50 μl of a solution consisting of 0.1% N-(1-naphthyl)ethylenediamine dihydrochloride was added and incubated for an additional 20 minutes at room temperature. The concentration of NO₂— was measured colourimetrically (λ550 nm) using a Tecan Safire2 microplate reader. A 0.1M sodium nitrite (NaNO₂) standard was used to make serial dilutions (1-100 μM) to quantify NO₂— by standard curve.

Neutral Red Phagocytosis Assay.

A 0.1% neutral red (NR) solution was prepared by dissolving 0.1 g NR crystals in 100 ml sterile phosphate buffer (pH7.2). The solution was then filtered using a syringe filter, thus removing and insoluble crystals and ensuring homogeneity. Assays were performed as described as described in Example 3. Post-incubation (24 h) with fractions SCAF0-SCAF5 (1 mg/ml-0.1 ng/ml), 20 μl of 0.1% NR solution was added to wells and incubated (6 h). Supernatants were carefully aspirated and discarded, and the wells washed twice with 200 μl PBS. Cells were lysed by addition 100 μl of cell lysis solution (50% abs.ethanol, 49% deionised water and 1% glacial acetic acid) cells were incubated at room temperature (2 h) and the optical density (540 nm) was measured using the Tecan Safire2 microplate reader.

LAL Assay (Endotoxin Detection).

The assay was performed in accordance to protocol provided by the manufacturers Pyrosate® (CapeCod), as described in Example 3.

TLR 4 Inhibition Assay TAK-242 (Resatorvid).

TAK-242 is a small molecule which is a specific inhibitor of Toll-like receptor (TLR) 4, which inhibits the production of pro-inflammatory mediators by binding to Cys747 in the intracellular domain of TLR4. This prevents interactions between TLR4 and its adaptor molecules Toll/IL-1 receptor domain containing adaptor protein (TIRAP) and Toll/IL-1 receptor domain-containing adaptor protein inducing interferon-β-related adaptor molecule (TRAM). Consequently, LPS-induced signal transduction is inhibited, including downstream signalling transduction that results in the production of pro-inflammatory cytokines (Matsunaga et al. 2011). TAK-242 is reported to have an IC₅₀ ranging from 1.1-11 nM (Ii et al. 2006). RAW 264.7 were prepared as described in Example 3 and subsequently seeded at a density of 1×10⁵ cells/well in two 96-well microtitre plates. S. cordifolia fractions (100 l) SCAF 0 and SCAF 5 (1-0.001 mg/ml) were added in triplicate to one of the plates. The positive control was 1 ng/ml LPS and the negative control was 100 μl DMEM medium. The remaining plate was arranged in an identical manner. However, the fractions SCAF 0 and SCAF 5 and positive (LPS 1 ng/ml) and negative (medium) all contained TAK-242 at a concentration 10 nM. Plates were incubated for 18 h in an incubator at 5% CO₂ at 37° C. After incubation, nitrite concentrations were determined using the Griess reagent.

Galleria mellonella

Galleria mellonella larvae (Livefoods Direct) were reared on an artificial diet 25° C. (dark), during experiments larvae were kept in an incubator at 37° C., in sterile Petri dishes (5 cm). Experimental groups consisted of 10 larvae (last instar) weighing 250-300 mg.

Immune Response in Galleria mellonella.

Fractions SCAF0 and SCAF5 at concentrations of 1 mg/ml, 0.1 mg/ml and 0.001 mg/ml were prepared in PBS in sterile conditions. Terumo Myjector 1 ml 29G 0.33×12 mm were used to inject 20 μl aliquots of extracts into the hemocoel through proleg (left) in dorsolateral region of larvae in each group, larvae were subsequently incubated for 24 h. Haemolymph was removed post incubation and haemocyte were enumerated using a haemocytometer.

Bacterial Load in Larvae Pre-Exposed to SCAF 0-SCAF5.

SCAF0 and SCAF5 (0.001, 0.1 and 1 mg/ml) and prepared in PBS under sterile conditions. A Terumo Myjector (1 ml 29G 0.33×12 mm) was used to inject 10 μl aliquots of extracts into the hemocoel through proleg (left) in dorsolateral region of larvae in each group, the negative control group was injected with PBS. Larvae were placed in an incubator set at 37° C. for 24 h. At 24 h after administration of aqueous extract, each group was infected with MRSA (ATCC 43300) inoculation experiments were carried out in accordance to protocol described by Ramarao et al. (2012). Inoculum density was standardised to the optical equivalent of the 0.5 McFarland turbidity standard, which at λ600 nm absorbance of 0.06-0.10 nm, corresponding to 107 cfu/ml (Andrews 2001), which was subsequently diluted in PBS producing an inoculum at a concentration of 1×105 CFU/ml. The larvae where then infected administered through a proleg (right) in dorsolateral region (injection volume 20 μl (2×103 CFU). Inoculated larvae were maintained in an incubator set at 37° C. for 24 h. Bacterial inoculums used to infect larvae were serially diluted to verify inoculum size using the Miles and Misra Method (Miles et al. 1938), which involves 20 μl of each dilution being plated on Muller-Hinton agar plates and incubated at 37° C. for 24 h. Inoculum concentration was calculated as 1.1×10⁵ CFU/ml. At 24 h, 48 h and 72 h post-infection 3 larvae from each group were removed and the bacterial load in haemolymph was determined by draining and diluting haemolymph. CFU/ml of haemolymph was determined by using the Miles and Misra Method to calculate CFU.

Effects on Bacterial Load in Larvae on Administering SC1 and SC2.

Larvae (10 larvae per group, weighing 250-300 mg) were infected with MRSA (ACTCC 4330) through the dorsolateral region through the left proleg (10 μl, 1.1×105 CFU/ml) using a 1 ml Terumo Myjector syringe (29G 0.33×12 mm) subsequently larvae were incubated for 6 h. Post-incubation larvae were administered compounds SC1 and SC2, larvae were subsequently maintained in an incubator at 37° C. for 48 h. Heamolymph was then removed and CFU/ml of haemolymph was determined by using the Miles and Misra Method to calculate CFU as described previously.

GC-MS Analysis

SCAF0 and SCAF 5 (100 mg) were hydrolysed with 10 ml 1M trifluroacetic acid (TFA) (Sigma-Aldrich) at 105° C. for 7 h in a closed 25 ml flask, (Uzaki & Ishiwatari 1983) and then lyophilised. Samples (2 mg) were derivitised by silylation reaction with 500 μl N,O-Bis(trimethylsilyl)trifluoroacetamide (BSTFA) with 1% trimethylchrosilane (Sigam-Aldrich) in 1 ml anhydrous pyridine. Reactions were carried out at room temperature for 12 h. GC-MS analysis was performed using gas chromatography (Agilent 7890A) interfaced with a mass selective detector (Agilents 5975C), with a ZB semi-volatiles column (30 m×0.25 mm×0.25 μm Zebron™, Phenomenex Inc) with helium as the carrier gas at a constant rate of 1 ml/min. The injector and MS source temperatures were maintained at 260° C. and 230° C., respectively. The column temperature program consisted of injection at 80° C. and hold for 1 min, temperature increase of 15° C. per min to 300° C., followed by an isothermal hold at 300° C. for 15 min. The MS was operated in the electron impact mode with an ionisation energy of 70 eV. The scan range was set from mass scan range was 50-550 Da. Injection volume was 1 μl, the inlet had a split flow of 20 ml-1 (split ratio 20:1). Data were acquired and processed with the Chemstation software (Hewlett Packard). Monomer identification was performed by comparing with chromatographic retention characteristics and mass spectra against standards, and the NIST mass spectral library (National Institute of Standards and Technology, USA).

Real Time Quantitative PCR (RT-qPCR).

Preparation of RNA Samples

A murine splenocytes cell suspension was prepared as described above. Cells were seeded at a density of 2×10⁶ cells/ml in a 6-well Corning® cell culture plate. Murine macrophages (RAW 264.7) were cultured as described in Example 3. Cells were seeded at a density of 5×10⁵ cells/ml. Subsequently, 1.5 ml S. cordifolia fractions SCAF 0 and SCAF 5 were added in triplicate to wells of seeded plates, at concentrations of 1 mg/ml and 0.1 mg/ml. The positive control was 1 ng/ml LPS and the negative control was 1.5 DMEM or RPMI medium. Plates were incubated at 37° C. for 24 h. Post-incubation, cells were gently removed from wells using a cell scraper and supernatants aspirated from plates and transferred to sterile universal tubes. Triplicates of each sample were pooled to the same universal tubes. The total number of cells was determined in each tube and subsequent adjustments were made consequently resulting in a final cell densities of 1.5×10⁶ cells/ml and 3×10⁵ cells/ml for splenocytes and RAW264.7 cells, respectively. Universal tubes were then centrifuged at 300 g for 15 min, supernatants being aspirated. RNA was isolated using the Qiagen RNeasy Mini kit (Qiagen LTD, Manchester, UK). Briefly, 600 μl RLT lysis buffer (a guanidinium thiocyanate-containing lysis buffer) (Qiagen) containing 1% v/v β-mercaptoethanol was added to universal tubes containing cell pellets and cells homogenised. The lysates were then transferred to RNAase-free Eppendorf tubes, tubes were then centrifuged at 10,000 g in a benchtop MiniSpin Eppendorf centrifuge (Eppendorf UK LTD, Stevenage, UK) minispin for 3 min. The supernatant was subsequently transferred to a 1.5 ml Eppendorf tube to which 600 μl 70% ethanol was added. Subsequently, 700 μl of sample was transferred to a spin column placed in a 2 ml collection tube and centrifuge for 1 min at 10000 g, thereupon remaining sample was passed through the spin column.

The spin column was then transferred to a clean collection column and filtrate discarded. 700 μl of RW1 buffer was then passed through spin column by centrifugation at 10000 g for 1 min, followed by 500 μl of RPE buffer and centrifugation at column at 10000 g, followed by an additional 500 μl of RPE buffer and centrifugation at 10000 g for 2 min, followed by an additional 1 min at 11000 g for 1 min. RNA was eluted from column membrane by adding 50 μl of RNase free water, followed by centrifugation at 10000 g.

Genomic DNA was removed from RNA samples by treating samples with TURBO™ DNase (Life Technologies) briefly the 10× TURBO DNase buffer (containing bivalent cations Ca2+ and Mg2+ essential for maximal activity of DNase enzyme) was added to a 1× concentration, followed by 1 μl TURBO DNase (2 U), tubes were incubated at 37° C. for 30 min. The purity of isolated RNA was assessed using the ratio of absorbance at 260 nm and 280 nm, was quantified using a NanoDrop 2000c spectrophotometer (Thermo Scientific, Loughborough, UK) the ratio was for samples was calculated as 2.0-2.1 for all samples. RNA samples were stored at −80° C., prior to cDNA synthesis.

Reverse Transcription, cDNA Synthesis.

RNA concentrations were quantified by absorbance at 260 nm using NanoDrop 2000c. The range was determined to be between 167.1 ng/μl and 208.8 ng/μl. Concentrations of RNA were adjusted to 150 ng/μl using RNase-free water prior to cDNA synthesis. Reverse transcription was carried out using the Transcriptor First-Strand cDNA synthesis kit (Roche Diagnostics Ltd, Sussex, UK) using the random hexamer primers, reverse transcription were carried out in triplicates in accordance to manufacturers protocol. RNA concentration used in all reactions was 1 μg. Transcription reaction was carried out on an Eppendorf Mastercycler. Reaction settings were as follows, 10 min at 25° C., followed by 30 min at 55° C., finally the reverse transcriptase was inactivated by heating to 85° C. for 5 min.

RT-qPCR

RT-qPCR was performed in accordance to the Minimum Information for Publication of Quantitative Real-Time PCR Experiments (MIQE) guidelines (Bustin et al. 2009). In order to determine reference gene for normalisation the stability of expression of gene must be unchanged under experimental factors (Kozera & Marcin 2013). Consequently, β-actin, β-2-microglobulin (B2M), glyceraldehyde-3-phosphate (GADPH), hydroxymethylbilane synthase (HMBS) and hypoxanthine guanine phosphoribosyl transferase 1 (HPRT1) were tested for stability. HPRT1 was identified to be the most stable gene, the expression of which remained unchanged and was consequently used as the reference gene, and used for normalisation of assays. Primers sequences with published efficiencies of 90-110% in a 2-step Sybr Green assay were identified and used to design primers. After amplification, melting curves were examined for primer-dimer artefacts and to ensure reaction specificity, electrophoresis of RT-PCR products in 1.0% agarose gel revealed the presence of amplicons of predicted size and the absence of non-specific amplification products.

TABLE 4
Primers (forward and reverse) used for assays.

| Gene | Forward | Reverse |
|---|---|---|
| IL-1a | GGAAGATTCTGAAGAAGAG | TGAGATTTTTAGAGTAA |
| | ACGG | CAGG |
| IL-1b | TGTCTGAAGCAGCTATGGC | CTGCCTGAAGCTCTTGT |
| | AAC | TGATG |
| IL-6 | TCTTGGGACTGATGCTGGT | CAGAATTGCCATTGCAC |
| | G | AACTC |
| IL-10 | AATTCCCTGGGTGAGAAGC | CATGGCCTTGTAGACAC |
| | TG | CTTG |
| 1L-12 | TGG ACC TGC CAG GTG | CAATGTGCTGGTTTGGT |
| p35 | TCT TAG | CCC |
| IL-12 | AAGAAGGAAAATGGAATTT | ATGTCACTGCCCGAGAG |
| p40 | GGTCC | TCAG |
| TNF- | CTCAGCCTCTTCTCATTCC | CCATAGAACTG |
| | TGC | ATGAGAGGG |
| IFN-γ | AGCAACAGC AAG GCG | CTGGACCTGTGGGTTGT |
| | AAA A | TGA |
| IFN-β | CGTGGGAGATGTCCTCAAC | AAGATCTCTGCTCGGAC |
| | T | CAC |
| iNOS | CAG CTG GGC TGT ACA | CAT TGG AAG TGA |
| | AAC CTT | AGC GTT TCG |
| (HPRT1) | GAGGAGTCCTGTTGATGTT | GGCTGGCCTATAGGCTC |
| | GCCAG | ATAGTGC |

PCR reaction was performed as 23 μl reaction, containing 100 ng cDNA template, 0.25 μM each of forward and reverse primers (see Table 4), 12.5 μl QuantiTect SYBR Green master mix (Qiagen LTD, Manchester, UK) 9.25 μl H20 (RNase-Free). Reactions were carried out on a LightCycler® 480 system (Roche Diagnostics Ltd, Sussex, UK) system under the following conditions: 45 cycles of denaturation at 94° C. for 15 see, annealing at 58° C. for 1 min and extension at 72° C. for 1 min. using a thermal. Electrophoresis of RT-PCR products in 1.0% agarose gel revealed the presence of amplicons of predicted size.

Quantitation of results were performed using the comparative Ct method, which involved normalising the gene of interest to the reference gene (HPRT1) followed comparing Ct values of treated samples (TRT) to those which act as a calibrator (CTL) (untreated cells), in the following manner (Pfaffl 2001).

ΔΔCt=ΔCt(TNFαTRT−HPRT1TRT)−ΔCt(TNFαCTL−HPRT1CTL)

Fold Change=2(−ΔΔCt)

Results

Lymphocyte Assays

Lymphocyte Proliferation Assay

Results from the proliferation assay preformed with the lymphocytes are summarised in FIG. 36. A concentration-dependent increase in stimulatory activity was evident with SCAF 3 and SCAF 5.

Antibody Secretion

Supernatants collected from wells with splenocytes incubated with Fractions of Sida cordifolia aqueous extract and antibody secretion measured. Results from the antibody secretion assays for IgG, IgM and IgA are presented in FIGS. 37, 38 and 39, respectively. A concentration-dependent increase in stimulatory activity was observed for SCAF 3 and SCAF 5.

Macrophage Assays (RAW 264.7).

Phagocytosis Assay.

Results for neutral red phagocytosis assay are presented in FIG. 40. A concentration-dependent increase in activity was observed with SCAF 5.

Nitric Oxide (NO) Production

The production of nitric oxide (NO) was detected, measured and quantified photometrically as the accumulation of its stable product nitrite in the culture supernatant using a colorimetric assay (the Griess reaction). Elevation of NO is a clear indicator of activation of the RAW264 macrophages cells. A concentration-dependent increase in activity was observed with SCAF 5 (FIG. 41 and FIGS. 51 and 52). The half-maximal concentration of polysaccharide for stimulation nitric oxide production in RAW 264.7 cells is about 8 μg/ml. In these cells the polysaccharide reaches a maximal response around 25 μg/ml.

Cytokine Production

The polysaccharide caused macrophages and microglia to release cytokines associated with an immune response. Macrophages produce IL-6 and TNF alpha in a concentration-dependent manner in response to contact with the polysaccharide according to the invention (see FIG. 53).

The polysaccharide also caused human umbilical vein endothelial cells (HUVECs) to produce or suppress cytokines associated with immune responses. HUVECs released IL-8 in response to contact with the polysaccharide and suppressed the release of MIG in response to contact with the polysaccharide according to the invention (see FIG. 54).

### DISCUSSION

This Example shows the purified extracts from S. cordifolia immunomodulatory activity. This dual activity makes S. cordifolia very unique and potentially very useful in treating and/or preventing infection. In this study we isolated and studied the source of the immunomodulatory activity. The immunomodulatory activity was identified to have originated from polysaccharide constituents.

Immunomodulation

Initial screening in Example 3 identified that the immunomodulatory activity originates from the aqueous extract, and upon further fractionation this activity was identified to have originated from the polysaccharide contained in the aqueous extract (SCAF 0). When comparing the activity of SCAF 0 to crude aqueous extract, SCAF 0 exhibited greater activity. In this Example, SCAF 0 was further fractionated by ion exchange chromatography. Fractions obtained from ion exchange chromatography were subsequently fractionated further by molecular size. It was found that the fraction, referred to as SCAF 5, had the greatest immunomodulatory activity. This stimulatory activity of SCAF 5 was observed to be stronger than the positive control lectin concanavalin A at 100 μg/ml. This observation is entirely supported by the observed increases in IgG, IgM and IgA levels, as well as in studies showing the stimulation of macrophage activation and phagocytosis. SCAF 5 has a high ionic strength and a molecular size greater than 100 kDa. Electrophoretic studies performed indicate that the polysaccharides range in size from 100 kDa-150 kDa. SCAF 2 and SCAF 4 with molecular weights ranging from 10-100 kDa exhibited more moderate activity profiles and therefore suggest that the size of the polysaccharide is an important determinant for the immunomodulatory activity. Although SCAF 4 triggered a response in the neutral red phagocytosis assay neither SCAF 2 or SCAF 4 significantly affected nitrite production in macrophages, thus conforming the weaker activity of these fractions. The potent activity of SCAF5 was tested in vivo in the Galleria model. A marked increase in haemocytes in haemolymph was observed with SCAF 5.

Furthermore, administration of SCAF 5 to the Galleria model assisted the larvae in preventing an MRSA infection being established. This effect was dose dependent.

SCAF 5-stimulated NO production in macrophages was corroborated by RT-qPCR analysis which showed a 2.1-fold increase in iNOS expression. Increases in expression of iNOS and NO production are indicative of a T_{H}1-mediated response (Lawrence & Hagemann 2012, Stuehr & Marletta 1985). The T_{H}1-mediated response contributes to the microbicidal (i.e. respiratory burst, by means of the production of reactive oxygen species). The activation of the T_{H}1 response was further supported by the observed increase in phagocytic activity in the macrophages. More conclusive evidence for a T_{H}1 mediated response was the finding that the expression of cytokines was increased in both RAW 264.7 cells and murine splenocytes. The expression of IL-6 a pro-inflammatory cytokine which is normally secreted by T-cells and activated macrophages was increased by 4.2- and 3.3-fold, respectively by SCAF 5 in splenocytes and macrophages.

Characteristic of a T_{H}1 mediated immune response, IL-6 is secreted by macrophages in response to PAMPs. The importance of IL-6 in infection has been demonstrated previously using an in vivo model in which C57Bl/6 mice lacking the IL-6 gene had an impaired defence to pneumococcal pneumonia (Streptococcus pneumonia) (Poll et al. 1997). Further evidence highly suggestive of SCAF 5 causing a T_{H}1 mediated immune response is the increase in expression of IL-12 which is involved in the differentiation of T_{H}0 (naïve T cells) into T_{H}1 cells, which consequently results in the production of IFN-γ and TNF-α from activated T cells and NK cells (Jacobson et al. 1995). SCAF 5 increased TNF-α by 1.2-fold and 2-fold in splenocytes and RAW 264.7 cells, respectively. TNF-α is produced by numerous types of cells including monocytes, macrophages, T-lymphocytes, B lymphocytes and NK cells. Characteristic of activated macrophages, TNF-α also participates wound healing, remodelling of tissues and has been shown angiogenesis (Leibovich et al. 1987) whilst promoting fibroblast proliferation (Battegay et al. 1995). TNF-α along with IL-1 and IL-6 have also been implicated in promoting the release of proteins from the liver which include the C-reactive protein which binds to phosphocholine expressed on microbes, the binding of which results in the activation of complement (Black et al. 2004) resulting in the elimination of pathogen. A 2.5-fold and 2.4-fold increase in IFN-γ and IFN-β, respectively was also observed by splenocytes in response to SCAF 5. As IFN-γ is characteristic of activated T lymphocytes and Natural Killer cells, this would indicate the presence of activated T cells and NK among the population of splenocytes and as IFN-γ has anti-proliferative and antiviral properties and is a powerful activator of mononuclear phagocytes, increasing their ability to destroy intracellular microorganisms (Huang et al. 1993). Mice lacking IFN-γ are killed by sub-lethal doses of pathogens including the intracellular pathogen, Mycobacterium bovis, which was shown to be lethal in mice even at sublethal doses (Dalton et al. 1993). Similar to IFN-γ, IFN-β also exhibits antiviral activities (Damme et al. 1987). 2.5-fold and 2.4-fold increases in the expression of IL-1β were also observed with SCAF 5 in both RAW 264.7 cells and splenocytes. Secreted by phagocytic cells IL-1 acts as a lymphocyte-activating factor, and as a lymphocyte mitogen, it is also responsible for the nonspecific resistance to pathogens. IL-1 has been shown to be suppressed by corticosteroids as are IL-12, IL-6, IFN-γ, IFN-β and TNF-α (Miyaura & Iwata 2002, Lew et al. 1988). The ability of SCAF 5 to increase these pro-inflammatory cytokines may warrant there use as a stimulant of the T_{H}1-mediated response. Further evidence for the proposed T_{H}1 mediated immune response comes from no change in the expression of IL-10 in both RAW 264.7 and splenocyte culture. The absence of endotoxins (LPS) in SCAF0-5 was confirmed by the LAL assay. Furthermore, the inability of TAK-242 to inhibit the activation of macrophages confirmed this as cells incubated in the presence of TAK-242 and LPS were inactivated. This suggests the pattern recognition receptor (PRRs) resulting in the activation of macrophages is not the TLR-4, and this activity is not due to the presence of endotoxin contaminants. GC-MS analysis of polysaccharides of SCAF 0 and SCAF 5 found that the monomer constituents were tsimilar but the ratio of sugars differed. SCAF5 contained higher levels of both uronic acid and glucuronic acid and this may may explain the need for a high ionic strength solution to elute SCAF 5. The increases proportion of mannose in SCAF 5 could implicate mannose receptors as mediators of immunomodulatory activity.

### Conclusion

This Example shows that the PP of Sida cordifolia exhibits immunomodulatory activity. Immunomodulatory activity is triggered by isolated polysaccharide molecules with molecular weights >100 kDa. No endotoxin was detected as confirmed by the LAL assay. The inability to prevent an immune response with TAK 242 confirms the immunomodulatory activity does not appear to be the result of direct activation of the TLR4 receptor. It is possible that immune activation is triggered by activation of mannose receptors since SCAF 5 has an increased proportion of mannose monosaccharides compared with the more weakly active fraction (SCAF1-4). The cytokine profile elicited by SCAF 5 is highly suggestive of a pro-inflammatory immune response mediated by T_{H}1 cells, where the immune system is primed for microbial attack. Increased iNOS in macrophage is also suggestive of a pro-inflammatory immune response.

### Example 5—Effect of Polysaccharide on Faecal Bacterial Diversity

Faecal samples from C57Bl/6J mice were assessed to determine its bacterial composition. Mice received daily oral gavage of either saline (0.9%), or saline containing the purified polysaccharide (300 mg/kg). Polysaccharide was prepared according to the ‘Strategy 3’ methodology described herein (and shown in FIG. 1). FIG. 55 shows the % composition of faecal microbiota (phylum level) in mice after 7 days of treatment with the polysaccharide or saline. FIG. 56 is a summary of the bacterial diversity (of FIG. 55) when analysed at the phylum level and the species level (alpha diversity was calculated using the Shannon index). FIGS. 55 and 56 clearly show that the polysaccharide according to the invention increases bacterial faecal diversity at the phylum level and species level in C57Bl/6J mice. Thus, the polysaccharide according to the invention may be used as a prebiotic.

What is claimed:

1-10. (canceled)

11. A method of isolating a polysaccharide from a plant from the Sida genus, the method comprising:
i. homogenising and dehydrating the plant, thereby forming dehydrated plant particles and/or powder; and
ii. extracting the polysaccharide from the dehydrated plant particles and/or powder, thereby isolating the polysaccharide from the plant.

12. The method according to claim 11, wherein homogenising the plant comprises grinding and/or chopping the plant into particles and/or a powder.

13. The method according to claim 11, wherein dehydrating the plant comprises lyophilising or heating the plant material to remove moisture.

14. The method according to claim 11, wherein extracting the polysaccharide from the dehydrated plant particles or powder comprises a first alcohol extraction step, followed by one or more aqueous extraction steps, followed by a second alcohol extraction step.

15. The method according to claim 14, wherein the second alcohol extraction step is performed on an aqueous phase of one or more aqueous extraction steps that have been pooled together, such that an alcohol phase, which contains the isolated polysaccharide, and a precipitate are formed.

16. The method according to claim 15, wherein the alcohol phase is separated from the precipitate by centrifugation.

17. The method according to claim 16, comprising a further purification step, such as a chromatographic technique, a crystallisation technique or a distillation technique.

18. A polysaccharide or a composition thereof produced by the method according to claim 11, wherein the polysaccharide comprises “n” repeating units, wherein each of the “n” repeating units comprises a backbone of alpha-(1-5)-linked arabinofuranose residues, a first side chain of a single alpha-arabinofuranose residue (1-2)-linked to an arabinofuranose residue of the backbone, and a second side chain of a single alpha-arabinofuranose residue (1-3)-linked to the same arabinofuranose residue of the backbone.

19-30. (canceled)

31. A method of treating, ameliorating or preventing a T helper cell (T_{H})-mediated disease or medical condition in a subject, the method comprising administering to the subject a polysaccharide, or a composition thereof, of claim 18, or a plant of the Sida genus or a part thereof.

32. The method of claim 31, wherein, the T helper cell (T_{H})-mediated disease or medical condition is a T_{H}1-mediated disease or medical condition, or a T_{H}2-mediated disease or medical condition.

33. The method of claim 32, wherein the T_{H}1-mediated disease or medical condition is one or more selected from the group comprising/consisting of rheumatoid arthritis (RA); psoriatic arthritis; psoriasis; inflammatory bowel syndrome (IBD); Crohn's disease; ulcerative colitis; multiple sclerosis (MS); flu, including pandemic flu; respiratory disorders, for example those caused by viruses, such as respiratory syncytial virus (RSV); cystic fibrosis (CF); herpes, including genital herpes; sepsis and septic shock; bacterial pneumonia; bacterial meningitis; dengue hemorrhagic fever; endometriosis; prostatitis; uveitis; uterine ripening; alopecia areata; ankylosing spondylitis; coeliac disease; dermatomyositis; diabetes mellitus Type 1; Goodpasture's syndrome; Graves' disease; Guillain-Barre syndrome; juvenile idiopathic arthritis; Hashimoto's thyroiditis; idiopathic thrombocytopenic purpura; Lupus erythematosus; mixed connective tissue disease; myasthenia gravis; narcolepsy; osteoarthritis; pemphigus vulgaris; pernicious anaemia; polymyositis; primary biliary cirrhosis; relapsing polychondritis; Sjogren's syndrome; temporal arteritis; vasculitis; Wegener's granulumatosis; age-related macular degeneration, an infectious disease; an autoimmune disorder; a cancer; post-cancer surgery or cancer treatment; and post-immunisation.

34. The method of claim 32, wherein the T_{H}2-mediated disease or medical condition is one or more selected from the group comprising/consisting of type 1 hypersensitivity disorders, including an allergy, asthma, eczema, hay fever, urticarial, chronic graft-versus-host disease, progressive systemic sclerosis, systemic lupus erythematosus; a chronic lung disease; scleroderma; anaphylaxis; atrophy; and transplant rejection.

35. The method of claim 31, wherein the polysaccharide, the composition or the plant, or the part of the plant, is administered orally, sublingually, topically, transdermally or parenterally.

36-38. (canceled)

39. The polysaccharide or the composition of claim 18, wherein the repeating units comprise block A, which is represented by Formula (I):

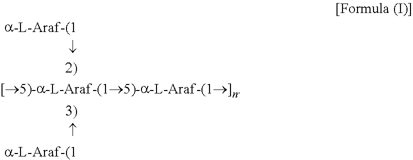

40. The polysaccharide or the composition of claim 18, wherein the repeating units further comprise block E, which is represented by Formula (II):

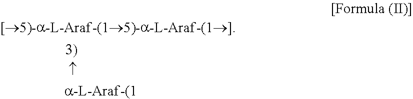

41. The polysaccharide or the composition of claim 18, wherein the repeating units further comprise block F, which is represented by Formula (III):

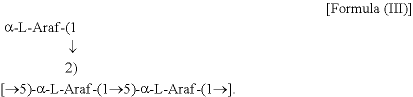

42. The polysaccharide or the composition of claim 39, wherein the backbone of each block is linked by an alpha-(1-5)-glycosidic bond.

43. The polysaccharide or the composition of claim 18, wherein the repeating units further comprise blocks A, E and F as defined by claims 39-41, and wherein:
a) the ratio of A:E:F in the repeating units is about 4-6:1-2:1;
b) the ratio of A:F in the repeating units is about 4-6:1; and/or
c) the ratio of A:E in the repeating units is about 2-6:1.

44. The polysaccharide or the composition of claim 18, wherein the repeating units comprise Formula (IV), defined herein as follows: 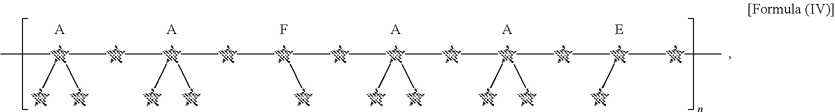
wherein each star of Formula (IV) corresponds to an arabinofuranose.

45. The polysaccharide or the composition of claim 18, wherein “n” is about 5 to about 1000.

46. The method of claim 11, wherein the plant from the Sida genus is Sida cordifolia.

47. The polysaccharide or the composition of claim 18, wherein the polysaccharide is an immunomodulatory agent or an immunological adjuvant.

48. A method of treating, ameliorating or preventing an infection by a microorganism in a subject, the method comprising administering to the subject a polysaccharide, or a composition thereof, of claim 18, or a plant of the Sida genus or a part thereof.
